(19) United States
(12) Patent Application Publication
(10) Pub. No.: US 2024/0217979 A1
(43) Pub. Date: Jul. 4, 2024

(54) GluN2 Subunit Selective Antagonists of the N-Methyl-D-Aspartate Receptors

(71) Applicant: Emory University, Atlanta, GA (US)

(72) Inventors: Dennis C. Liotta, Atlanta, GA (US); Stephen F. Traynelis, Decatur, GA (US); David S. Menaldino, Atlanta, GA (US); Peipei Ma, Atlanta, GA (US)

(21) Appl. No.: 18/287,844

(22) PCT Filed: Apr. 21, 2022
(86) PCT No.: PCT/US2022/025715

§ 371 Date: Oct. 20, 2023

Related U.S. Application Data
(60) Provisional application No. 63/177,430, filed on Apr. 21, 2021.

(51) Int. Cl. C07D 487/04; A61K 31/415; A61K 31/4365; A61K 31/437; A61K 31/4375; A61K 31/4439; A61K 31/4709; A61K 31/5025; A61K 31/519; C07D 231/06; C07D 401/04; C07D 401/14; C07D 413/14; C07D 417/14; C07D 471/04; C07D 495/04; C07D 513/04

(52) U.S. Cl. CPC C07D 487/04; A61K 31/415; A61K 31/4365; A61K 31/437; A61K 31/4375; A61K 31/4439; A61K 31/4709; A61K 31/5025; A61K 31/519; C07D 231/06; C07D 401/04; C07D 401/14; C07D 413/14; C07D 417/14; C07D 471/04; C07D 495/04; C07D 513/04

(57) ABSTRACT

Compounds that selectively inhibit the GluN2 subunits of NMDA receptors are disclosed. In general, the compounds have superior activity against GluN2C/D subunit(s) over GluN2A/B subunit(s). Optionally, the compounds have a structure of Formulas (I), (II), (III), or other formulas disclosed herein, enantiomers or diastereomers thereof, or pharmaceutically acceptable salts thereof. Pharmaceutical formulations containing one or more of the compounds are also disclosed. Additionally, methods of treating a neurological condition or disorder using the compounds or their pharmaceutical formulations thereof are disclosed. Exemplary neurological conditions or disorders include Parkinson's disease, dystonia and related motor disorders, depression, neuropsychiatric indications, stoke, hypoxia, traumatic brain injury, acute injuries involving the white matter, Alzheimer's disease, compulsivity and related disorders, and epilepsy.

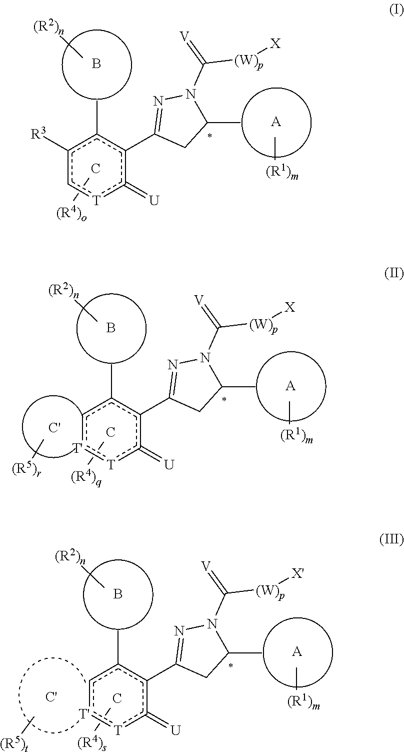

### CROSS-REFERENCE TO RELATED APPLICATIONS

This application claims the benefit of U.S. Provisional Application No. 63/177,430 filed Apr. 21, 2021, the entirety of which is hereby incorporated by reference for all purposes.

### STATEMENT REGARDING FEDERALLY SPONSORED RESEARCH OR DEVELOPMENT

This invention was made with government support under NS065371 and NS111619 awarded by the National Institutes of Health. The government has certain rights in the invention.

### TECHNICAL FIELD

The present disclosure relates to N-methyl-D-aspartic acid receptor (NMDAR) modulators and, in particular, to subunit specific allosteric modulators of NMDARs for the treatment of neurological disorders or conditions, such as Alzheimer's disease, Parkinson's disease, schizophrenia, epilepsy, depression, stroke, psychosis, and the like. It also relates to pharmaceutical compositions and methods of treatment of such neurological disorders or conditions involving the NMDAR modulators.

### BACKGROUND

N-methyl-D-aspartate receptors (NMDARs) belong to the family of ionotropic glutamate receptors that mediate excitatory neurotransmission in the mammalian central nervous system (CNS). NMDAR dysfunction has been implicated in neurological disorders such as Parkinson's disease, schizophrenia, and depression (Zhou, et al., Neuropharmacology, 2013, 74:69-75; Hallett, et al., Pharmacol. Ther., 2004, 102(2):155-74; Glasgow, et al., J. Physiol., 2015, 593(1):83-95; Mota, et al., Neuropharmacology, 2014, 76:16-26).

The functional NMDARs are heterotetramers and are assembled from two glycine-binding GluN1 subunits with either two glutamate-binding GluN2 (A-D) subunits or a combination of one GluN2 and one GluN3. Each subunit is comprised of four semiautonomous domains: the amino terminal domain (NTD), the agonist binding domain (ABD), the transmembrane domain (TMD), and the carboxyl terminal domain (CTD). The GluN2 subunits are encoded by four different genes, which give rise to GluN2A, GluN2B, GluN2C, and GluN2D subunits. These four subunits show different spatiotemporal expression patterns in the brain and determine the distinct physiological processes associated with these receptors. Therefore, the development of subunit-selective modulators is of great therapeutic interest in treating diseases associated with dysfunction of NMDARs.

The subthalamic nucleus (STN) is a key component of the basal ganglia, a group of subcortical nuclei that controls movement and are dysregulated in movement disorders like Parkinson's disease (PD) (Swanger, et al., Journal of Neuroscience, 2015, 35(48):15971). GluN2D-containing receptors expressed in subthalamic neurons participate in excitatory synaptic activity onto STN neurons. In the parkinsonian condition, degeneration of dopaminergic neurons in the substantia nigra pars compacta (SNc) leads to an imbalance of the output of several different nuclei, including potential for over excitation of glutamatergic neurotransmission pathway from the STN (Zhang, et al., Br. J. Pharmacol., 2014, 171(16):3938-45). This dysfunction can be improved by a potent and GluN2D subunit-selective antagonist, thereby improving clinical symptoms of movement disorder and benefiting patients suffering from related neurological disorders.

While subunit selective compounds have been developed that inhibit GluN2A- (prototype TCN-201) and GluN2B-containing NMDARs (prototype ifenprodil), compounds that target the GluN2C and GluN2D receptors are currently underdeveloped. Furthermore, structural basis for the action of existing GluN2C/D-selective compounds is not well understood due to the difficulty in the crystallization of these receptors compared to GluN2A/2B (Wang, et al., Nat. Commun., 2020, 11(1):423).

There is an unmet need for GluN2 subunit selective NMDAR modulators, especially for GluN2C/D subunit selective NMDAR modulators, with improved pharmaceutical properties.

### SUMMARY

Disclosed are negative allosteric modulators that selectively inhibit the GluN2 subunits of NMDARs. In some cases, the modulators selectively inhibit GluN2C and/or GluN2D over GluN2A and/or GluN2B.

In some embodiments, the disclosed compounds contain a monocyclic core (i.e., ring C), as shown in Formula I, an enantiomer or diastereomer thereof, or a pharmaceutically acceptable salt thereof,

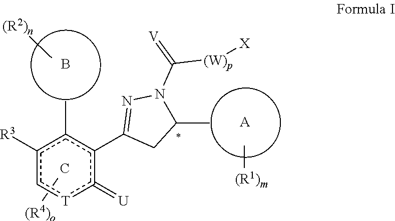

wherein ring A and ring B are independently 5- or 6-membered aryl or heteroaryl;
wherein T is CH, CR⁴, CH₂, CHR⁴, C(R⁴)₂, N, NH, or NR⁴;
wherein U is O, S, or absent;
wherein V is O, S, NH, N-alkyl, two hydrogen atoms each singly bonded to the adjacent carbon, or two fluorine atoms each singly bonded to the adjacent carbon;
wherein W, at each occurrence, is independently CH₂, C(Hal)₂, CH₂CH₂, CH₂CH₂CH₂, C(Hal)₂CH₂, CH₂C(Hal)₂, CH═CH, C(Hal)=CH, CH═C(Hal), C═O, O, S, or NH, optionally substituted by one or more, the same or different R¹⁰;
wherein p is 1, 2, or 3;
wherein X is —C(═Y)Z or 5- or 6-membered nitrogen-containing heterocyclyl optionally substituted by one or more, the same or different R⁶;
wherein Y is O, S, NH, N-alkyl, two hydrogen atoms each singly bonded to the adjacent carbon, or two fluorine atoms each singly bonded to the adjacent carbon;
wherein Z is halogen, OH, O-alkyl, O-alkanoyl, NH₂, NH-alkyl, N(alkyl)₂, NH-alkanoyl, N[(alkyl)(alkanoyl)], SH, S-alkyl, or S-alkanoyl, optionally substituted with one or more, the same or different R¹⁰;
wherein m and n are independently 0, 1, 2, 3, 4, or 5;
wherein o is 0, 1, 2, or 3;
wherein R¹, R², R⁴, and R⁶, at each occurrence, are individually and independently alkyl, haloalkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heteroaryl, or heterocyclyl, optionally substituted with one or more, the same or different R¹⁰;
wherein R³ is alkyl or haloalkyl, optionally substituted with one or more, the same or different R¹⁰;
wherein R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl; and
wherein the dotted lines in ring C each independently represent a single bond (i.e., a pair of shared electrons) or no bond (i.e., void).

In some embodiments, the compounds have a structure of Formula IA, an enantiomer or diastereomer thereof, or a pharmaceutically acceptable salt thereof,

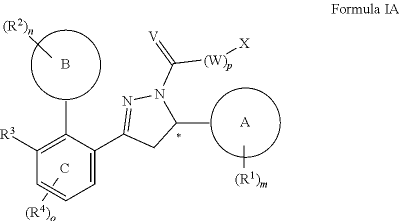

wherein o is 0, 1, 2, or 3; and
wherein ring A, ring B, V, W, X, R¹, R², R³, R⁴, m, n, and p are the same as those described for Formula I.

In some embodiments, the compounds have a structure of Formula IB, an enantiomer or diastereomer thereof, or a pharmaceutically acceptable salt thereof,

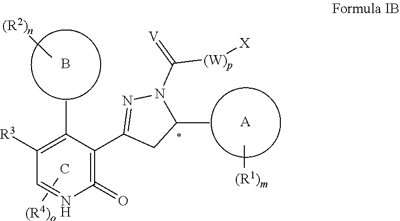

wherein o is 0, 1, or 2; and
wherein ring A, ring B, V, W, X, R¹, R², R³, R⁴, m, n, and p are the same as those described for Formula I.

In some embodiments, ring A and ring B are phenyl, for Formula I, IA, or IB.

In some embodiments, V is O, for Formula I, IA, or IB.

In some embodiments, p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰, for Formula I, IA, or IB.

In some embodiments, X is —C(═Y)Z, for Formula I, IA, or IB. Optionally, Y is O, for Formula I, IA, or IB. Optionally, Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰, for Formula I, IA, or IB.

In some embodiments, X is 5- or 6-membered nitrogen-containing heterocyclyl optionally substituted by one or more, the same or different R⁶, for Formula I, IA, or IB. Optionally, the 5- or 6-membered nitrogen-containing heterocyclyl is tetrazole.

In some embodiments, m is 1 and R¹ is halogen, for Formula I, IA, or IB. In some embodiments, n is 1 and R² is halogen, for Formula I, IA, or IB. In some embodiments, o is 1 and R⁴ is C₁-C₄ alkyl or C₁-C₄ haloalkyl, such as methyl, for Formula I, IA, or IB.

In some embodiments, R³ is C₁-C₄ alkyl or C₁-C₄ haloalkyl, such as methyl, for Formula I, IA, or IB.

In some embodiments, the compounds have the following features:
ring A and ring B are phenyl;
V is O;
p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰;
X is —C(═O)Z or 5- or 6-membered nitrogen-containing heterocyclyl optionally substituted by one or more, the same or different R⁶; Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰; m and n are independently 0, 1, or 2;
o is 0 or 1; and
the other undefined chemical groups are the same as those described above for Formula I, IA, or IB.

In some embodiments, the monocyclic-core compounds are selected from:

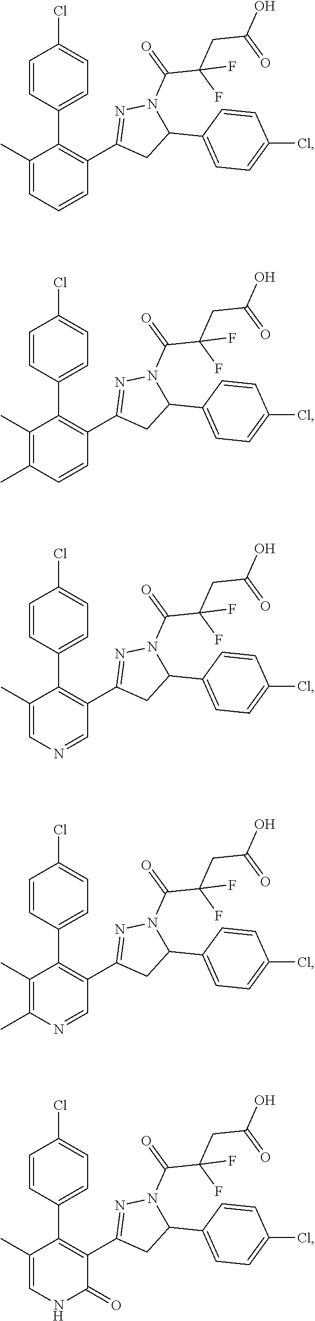

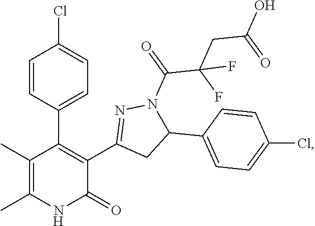

and pharmaceutically acceptable salts thereof.

In some embodiments, the disclosed compounds contain a bicyclic core (i.e., ring C fused with ring C′), as shown in Formula II, an enantiomer or diastereomer thereof, or a pharmaceutically acceptable salt thereof,

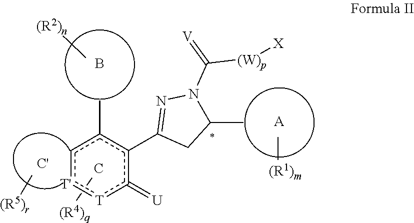

wherein ring A and ring B are independently 5- or 6-membered aryl or heteroaryl;
wherein ring C′ is 5- or 6-membered heteroaryl;
wherein T is CH, CR⁴, CH₂, CHR⁴, C(R⁴)₂, N, NH, or NR⁴;
wherein T′ is C or N;
wherein U is O, S, or absent;
wherein V is O, S, NH, N-alkyl, two hydrogen atoms each singly bonded to the adjacent carbon, or two fluorine atoms each singly bonded to the adjacent carbon;
wherein W, at each occurrence, is independently CH₂, C(Hal)₂, CH₂CH₂, CH₂CH₂CH₂, C(Hal)₂CH₂, CH₂C(Hal)₂, CH═CH, C(Hal)=CH, CH═C(Hal), C═O, O, S, or NH, optionally substituted by one or more, the same or different R¹⁰;
wherein p is 1, 2, or 3;
wherein X is —C(═Y)Z or 5- or 6-membered nitrogen-containing heterocyclyl optionally substituted by one or more, the same or different R⁶;
wherein Y is O, S, NH, N-alkyl, two hydrogen atoms each singly bonded to the adjacent carbon, or two fluorine atoms each singly bonded to the adjacent carbon;
wherein Z is halogen, OH, O-alkyl, O-alkanoyl, NH₂, NH-alkyl, N(alkyl)₂, NH-alkanoyl, N[(alkyl)(alkanoyl)], SH, S-alkyl, or S-alkanoyl, optionally substituted with one or more, the same or different R¹⁰;
wherein m and n are independently 0, 1, 2, 3, 4, or 5;
wherein q is 0, 1, or 2;
wherein r is 0, 1, 2, or 3;
wherein R¹, R², R⁴, R⁵, and R⁶, at each occurrence, are individually and independently alkyl, haloalkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heteroaryl, or heterocyclyl, optionally substituted with one or more, the same or different R¹⁰;
wherein R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl; and
wherein the dotted lines in ring C each independently represent a single bond (i.e., a pair of shared electrons) or no bond (i.e., void).

In some embodiments, ring C′ is 5-membered heteroaryl, such as thiophene, pyrrole, pyrazole, oxathiole, isoxathiole, thiazole, or isothiazole. Optionally, ring C′ is thiophene.

In some embodiments, ring C′ is 6-membered heteroaryl, such as pyridine or diazine.

Optionally, ring C′ is pyridine.

In some embodiments, the compounds have a structure of Formula IIA, an enantiomer or diastereomer thereof, or a pharmaceutically acceptable salt thereof,

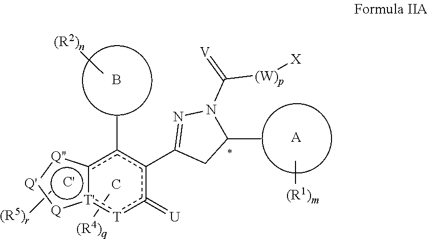

wherein Q, Q′, and Q″ are independently selected from S, O, N, NH, NR⁵, CH, or CR⁵;
wherein at least one of Q, Q′, and Q″ contains a heteroatom;
wherein r is 0, 1, or 2; and
wherein ring A, ring B, T, T′, U, V, W, X, R¹, R², R⁴, R⁵, m, n, p, and q are the same as those described for Formula II.

It is understood that the value of r in Formula IIA represents the number of incidences in which any of Q, Q′, and Q″ is NR⁵ or CR⁵.

In some embodiments, ring A and ring B are phenyl, for Formula II or IIA.

In some embodiments, V is O, for Formula II or IIA.

In some embodiments, p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰, for Formula II or IIA.

In some embodiments, X is —C(═Y)Z, for Formula II or IIA. Optionally, Y is O, for Formula II or IIA. Optionally, Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰, for Formula II or IIA.

In some embodiments, X is 5- or 6-membered nitrogen-containing heterocyclyl optionally substituted by one or more, the same or different R⁶, for Formula II or IIA. Optionally, the 5- or 6-membered nitrogen-containing heterocyclyl is tetrazole.

In some embodiments, m is 1 and R¹ is halogen, for Formula II or IIA. In some embodiments, n is 1 and R² is halogen, for Formula II or IIA. In some embodiments, q is 0, for Formula II or IIA. In some embodiments, r is 0, for Formula II or IIA.

In some embodiments, the compounds have the following features:
ring A and ring B are phenyl;
V is O;
p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰;
X is —C(═O)Z or 5- or 6-membered nitrogen-containing heterocyclyl optionally substituted by one or more, the same or different R⁶;
Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰;
m and n are independently 0, 1, or 2;
q is 0 or 1;
r is 0, 1, or 2; and
the other undefined chemical groups are the same as those described above in Formula II or IIA.

In some embodiments, the bicyclic-core compounds are selected from:

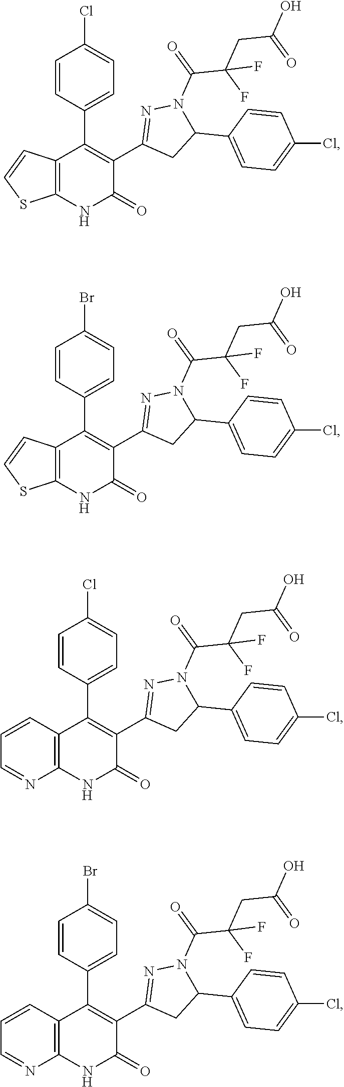

and pharmaceutically acceptable salts thereof.

In some embodiments, the disclosed compounds have a structure as shown in Formula III, an enantiomer or diastereomer thereof, or a pharmaceutically acceptable salt thereof,

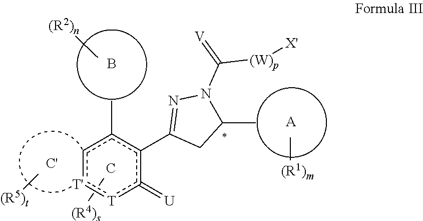

wherein ring A and ring B are independently 5- or 6-membered aryl or heteroaryl;
wherein ring C′ is 5- or 6-membered aryl or heterocyclyl or absent;
wherein T is CH, CR⁴, CH₂, CHR⁴, C(R⁴)₂, N, NH, or NR⁴;
wherein T′ is C or N when ring C′ is present and is CH, CR⁴, CH₂, CHR⁴, C(R⁴)₂, N, NH, or NR⁴ when ring C′ is absent;
wherein U is O, S, or absent;
wherein V is O, S, NH, N-alkyl, two hydrogen atoms each singly bonded to the adjacent carbon, or two fluorine atoms each singly bonded to the adjacent carbon;
wherein W, at each occurrence, is independently CH₂, C(Hal)₂, CH₂CH₂, CH₂CH₂CH₂, C(Hal)₂CH₂, CH₂C(Hal)₂, CH═CH, C(Hal)=CH, CH═C(Hal), C═O, O, S, or NH, optionally substituted by one or more, the same or different R¹⁰;
wherein p is 1, 2, or 3;
wherein X′ is 5- or 6-membered nitrogen-containing heterocyclyl optionally substituted by one or more, the same or different R⁶;
wherein m and n are independently 0, 1, 2, 3, 4, or 5;
wherein s is 0, 1, 2, 3, or 4 when ring C′ is absent and is 0, 1, or 2 when ring C′ is present;
wherein t is 0 when ring C′ is absent and is 0, 1, 2, 3, or 4 when ring C′ is present;
wherein R¹, R², R⁴, R⁵, and R⁶, at each occurrence, are individually and independently alkyl, haloalkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heteroaryl, or heterocyclyl, optionally substituted with one or more, the same or different R¹⁰;
wherein R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl; and
wherein the dotted lines in ring C each independently represent a single bond (i.e., a pair of shared electrons) or no bond (i.e., void).

In some embodiments, ring C′ is present in Formula III. In some embodiments, ring C′ is 5-membered heteroaryl, such as thiophene, pyrrole, pyrazole, oxathiole, isoxathiole, thiazole, and isothiazole. For example, ring C′ is thiophene. In some embodiments, ring C′ is 6-membered aryl or heteroaryl, such as phenyl, pyridine, or diazine. For example, ring C′ is phenyl.

In some embodiments, the compounds have a structure of Formula IIIA, an enantiomer or diastereomer thereof, or a pharmaceutically acceptable salt thereof,

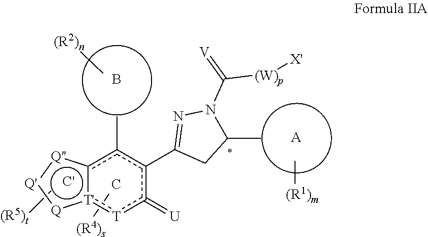

wherein Q, Q′, and Q″ are independently selected from S, O, N, NH, NR⁵, CH, and CR⁵;
wherein at least one of Q, Q′, and Q″ contains a heteroatom;
wherein t is 0, 1, 2, or 3;
wherein s is 0, 1, or 2; and
wherein ring A, ring B, T, T′, U, V, W, X′, R¹, R², R⁴, R⁵, m, n, and p are the same as those described for Formula III.

It is understood that the value of t in Formula IIIA represents the number of incidences in which any of Q, Q′, and Q″ is NR⁵ or CR⁵.

In some embodiments, Q is S, Q′ is CH or CR⁵, and Q″ is CH or CR⁵.

In some embodiments, the compounds have a structure of Formula IIIB, an enantiomer or diastereomer thereof, or a pharmaceutically acceptable salt thereof,

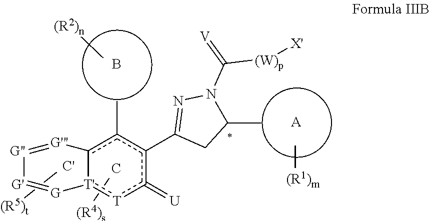

wherein G, G′, G″, and G′″ are independently CH, CR⁵ or N;
wherein t is 0, 1, 2, 3, or 4;
wherein s is 0, 1, or 2; and
wherein ring A, ring B, T, T′, U, V, W, X′, R¹, R², R⁴, R⁵, m, n, and p are the same as those described for Formula III.

It is understood that the value of t in Formula IIIB represents the number of incidences in which any of G, G′, G″, and G′″ is CR⁵.

In some embodiments, G, G′, G″, and G′″ are independently CH or CR⁵.

In some embodiments, ring C′ is absent in Formula III.

For example, the compounds have a structure of Formula IIIC, an enantiomer or diastereomer thereof, or a pharmaceutically acceptable salt thereof,

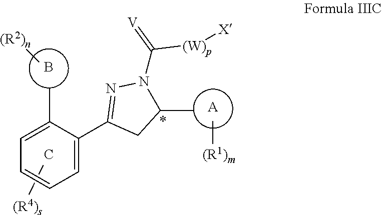

wherein s is 0, 1, 2, 3, or 4; and
wherein ring A, ring B, V, W, X′, R¹, R², R⁴, m, n, and p are the same as those described for Formula III.

In another example, the compounds have a structure of Formula HID, an enantiomer or diastereomer thereof, or a pharmaceutically acceptable salt thereof,

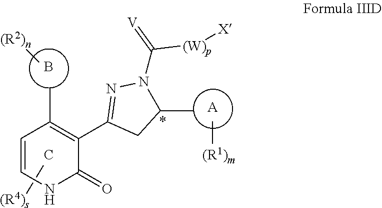

wherein s is 0, 1, 2, or 3; and
wherein ring A, ring B, V, W, X′, R¹, R², R⁴, m, n, and p are the same as those described for Formula III.

In another example, the compounds have a structure of Formula IIIE, an enantiomer or diastereomer thereof, or a pharmaceutically acceptable salt thereof,

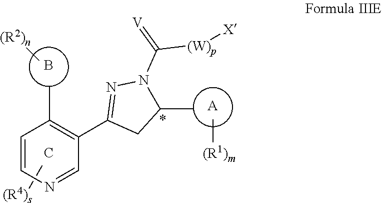

wherein s is 0, 1, 2, or 3; and
wherein ring A, ring B, V, W, X′, R¹, R², R⁴, m, n, and p are the same as those described above in Formula III.

In some embodiments, the 5- or 6-membered nitrogen-containing heterocyclyl in X′ for Formula III, IIIA, IIIB, IIIC, HID, or IIIE is selected from tetrazole (such as 1-tetrazole, 2-tetrazole, and 5-tetrazole), imidazole (such as 2-imidazole and 4-imidazole), oxazole (such as 2-oxazole and 4-oxazole), triazole (such as 3-(1,2,4-triazole)), thiazole (such as 2-thiazole and 4-thiazole), thiazolidine dione, oxazolidine dione, oxadiazol-5(4H)-one, thiadiazol-5(4H)-one, oxathiadiazole-2-oxide, and oxadiazol-5(4H)-thione. For example, the 5- or 6-membered nitrogen-containing heterocyclyl is tetrazole.

Optionally, the 5- or 6-membered nitrogen-containing heterocyclyl in X′ for Formula III, IIIA, IIIB, IIIC, HID, or IIIE is selected from the following:

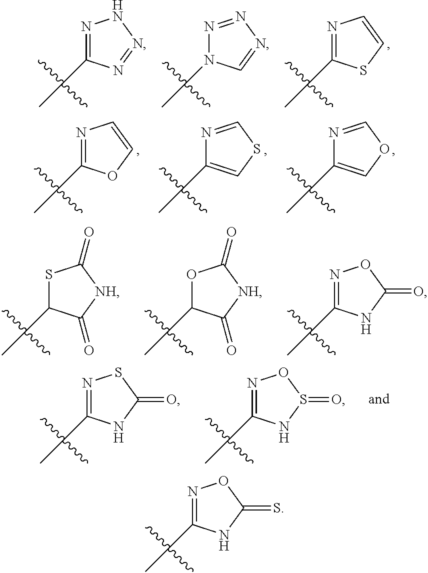

In some embodiments, X′ is 5-membered nitrogen-containing heterocyclyl optionally substituted by one or more, the same or different R⁶, for Formula III, IIIA, IIIB, IIIC, IIID, or IIIE. For example, X′ is 5-membered nitrogen-containing heteroaryl optionally substituted by one or more, the same or different R⁶.

In some embodiments, the compounds have a structure of Formula IIIF or a pharmaceutically acceptable salt thereof,

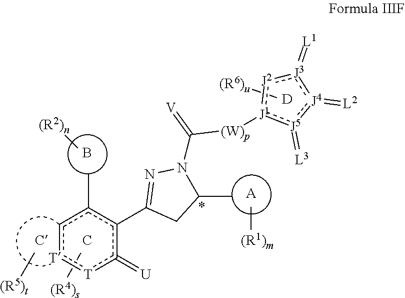

wherein u is 0, 1, 2, 3, or 4;
wherein J¹, J², J³, J⁴, and J⁵ are independently selected from S, O, N, NH, NR⁶, C, CH, and CR⁶;
wherein at least one of J¹, J², J³, J⁴, and J⁵ is N, NH, or NR⁶ and at least one of J¹, J², J³, J⁴ and J⁵ is C, CH, or CR⁶;
wherein L¹, L², and L³ are independently 0, S, or absent;
wherein when L¹ is not absent, J³ is C or S;
wherein when L² is not absent, J⁴ is C or S;
wherein when L³ is not absent, J⁵ is C or S; and
wherein the dotted lines in ring D each independently represent a single bond (i.e., a pair of shared electrons) or no bond (i.e., void).

It is understood that the value of u in Formula IIIF represents the number of incidences in which any of J¹, J², J³, J⁴, and J⁵ is NR⁶ or CR⁶.

In some embodiments, u is 0.

In some embodiments, L¹, L², and L³ are absent. In some embodiments, L₁ and L³ are absent and L² is 0 or S. In some embodiments, L¹ and L³ are independently 0 or S and L² is absent.

In some embodiments, ring A and ring B are phenyl, for Formula III, IIIA, IIIB, IIIC, HID, IIIE, or IIIF.

In some embodiments, V is O, for Formula III, IIIA, IIIB, IIIC, HID, IIIE, or IIIF.

In some embodiments, p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰, for Formula III, IIIA, IIIB, IIIC, IIID, IIIE, or IIIF.

In some embodiments, m is 1 and R¹ is halogen, for Formula III, IIIA, IIIB, IIIC, HID, IIIE, or IIIF. In some embodiments, n is 1 and R² is halogen, for Formula III, IIIA, IIIB, IIIC, HID, IIIE, or IIIF.

In some embodiments, the compounds have the following features:
ring A and ring B are phenyl;
V is O;
p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰;
m and n are independently 0, 1, or 2; and
the other undefined chemical groups are the same as those described for Formula III, IIIA, IIIB, IIIC, HID, IIIE, or IIIF.

In some embodiments, the compounds are selected from:

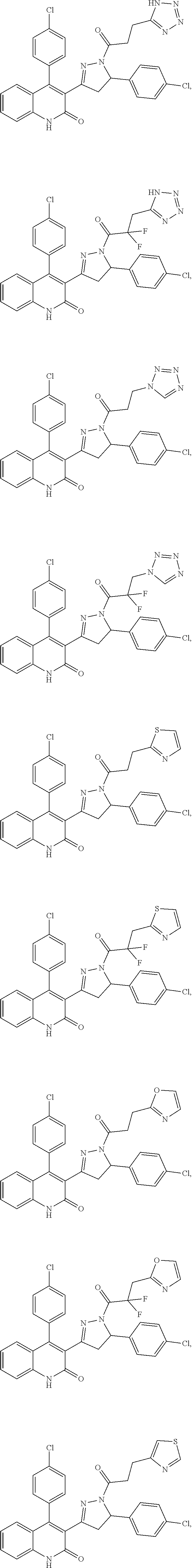

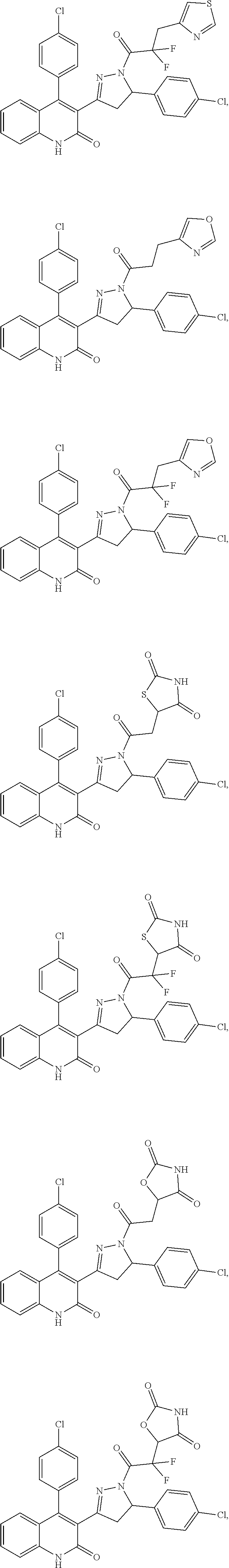

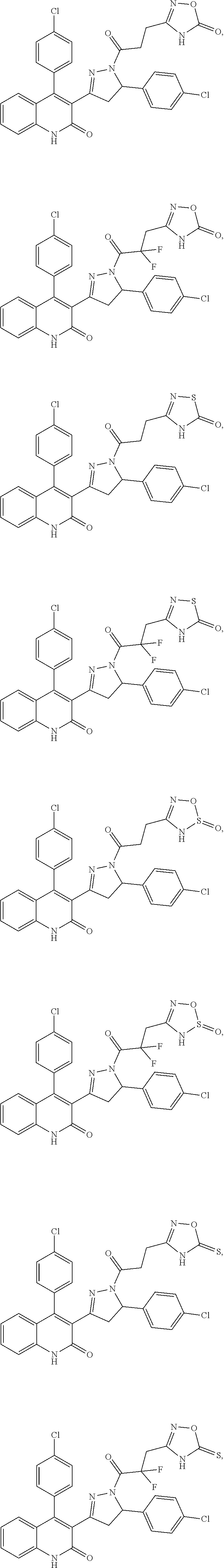

and pharmaceutically acceptable salts thereof,

Also disclosed are compositions containing a compound described herein, wherein the compound is in greater than 60%, 70%, 80%, 90%, 95%, or 98% enantiomeric excess with respect to the stereocenter labeled by the “*” sign in the formulas disclosed herein.

In some embodiments, the compound of Formula I in its corresponding composition is in greater than 95% enantiomeric excess for the configuration depicted below:

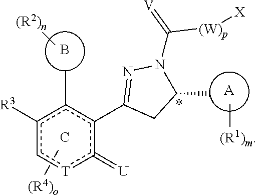

In some embodiments, the compound of Formula II in its corresponding composition is in greater than 95% enantiomeric excess for the configuration depicted below:

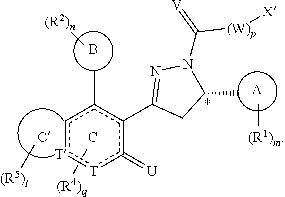

In some embodiments, the compound of Formula III in its corresponding composition is in greater than 95% enantiomeric excess for the configuration depicted below:

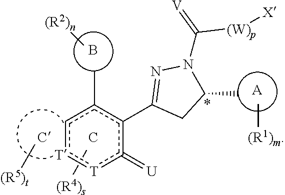

Also disclosed are pharmaceutical formulations of the disclosed compounds or compositions. In general, the pharmaceutical formulations also contain a pharmaceutically acceptable carrier. In some embodiments, the pharmaceutical formulations are in the form of tablet, capsule, pill, gel, cream, granule, solution, suspension, emulsion, or nanoparticulate formulation. In some embodiments, the pharmaceutical formulations are oral formulations. In some embodiments, the pharmaceutical formulations are intravenous formulations.

This disclosure also relates to (1) the compounds disclosed herein for treatment of a neurological condition or disorder disclosed herein or use as a medicament, (2) the compounds disclosed herein for use in the treatment of a neurological condition or disorder disclosed herein, or (3) the compounds disclosed herein for the manufacture of a medicament for treatment of a neurological condition or disorder disclosed herein.

This disclosure also provides methods of treating a neurological condition or disorder in a subject in need thereof. The methods include administering an effective amount of a compound disclosed herein to the subject. In some embodiments, the neurological condition or disorder is mediated by GluN2C- or GluN2D-containing NMDARs.

### DETAILED DESCRIPTION

The present disclosure describes negative allosteric modulators that selectively inhibit the GluN2 subunits of NMDARs, especially the GluN2C and/or GluN2D subunits. In some cases, the modulators selectively inhibit GluN2C and/or GluN2D over GluN2A and/or GluN2B. For example, the IC₅₀ values of the modulators for GluN2C and/or GluN2D are lower than the corresponding IC₅₀ values for GluN2A and/or GluN2B.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. To the extent that chemical formulas reported herein contain one or more unspecified chiral centers, the formulas are intended to encompass all stable stereoisomers, enantiomers, and diastereomers. It is also understood that the formulas encompass all tautomeric forms.

### I. Definitions

It must be noted that, as used in the specification, the singular forms “a,” “an,” and “the” include plural referents unless the context clearly dictates otherwise.

“Subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments of the present disclosure also contemplate treatment that merely reduces symptoms and/or delays disease progression. For example, the disclosure encompasses treatment that reduce the symptoms of or cognitive deficits associated with a neurological disorder or condition described herein.

As used herein, the term “combination with” when used to describe administration with an additional treatment means that the agent may be administered prior to, together with, or after the additional treatment, or a combination thereof.

As used herein, “alkyl” means a noncyclic straight chain or branched chain, unsaturated or saturated hydrocarbon such as those containing from 1 to 25 carbon atoms. For example, a “C₈-C₁₈ alkyl” refers to an alkyl containing 8 to 18 carbon atoms. Likewise, a “C₆-C₂₂ alkyl” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; representative saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; representative straight chain and branched alkynyls include acetylenyl, propynyl, 1-butynyl, 2-butynyl, 1-pentynyl, 2-pentynyl, 3-methyl-1-butynyl, and the like.

As used herein, “heteroalkyl” refers to alkyl groups where one or more carbon atoms are replaced with a heteroatom, such as, O, N, or S. Similar to alkyl groups, heteroalkyl groups can be straight or branched, saturated or saturated. Optionally, the nitrogen and/or sulphur heteroatom(s) can be oxidized, and the nitrogen heteroatom(s) can be quaternized. Suitable heteroalkyl groups may contain 1 to 25 carbon atoms and 1 to 4 heteroatoms.

Non-aromatic, mono or polycyclic alkyls are referred to as “carbocycles” or “carbocyclyl” groups. Representative saturated carbocycles include cyclopropyl, cyclobutyl, cyclopentyl, cyclohexyl, and the like; representative unsaturated carbocycles include cyclopentenyl, cyclohexenyl, and the like. In polycyclic carbocyclyl groups, the rings can be attached together in a pendant manner (i.e., two rings are connected by a single bond), in a spiro manner (i.e., two rings are connected through a defining single common atom), in a fused manner (i.e., two rings share two adjacent atoms—in other words, two rings share one covalent bond), in a bridged manner (i.e., two rings share three or more atoms, separating the two bridgehead atoms by a bridge containing at least one atom), or a combination thereof. The number of “members” of a carbocyclyl group refers to the total number of carbon atoms in the ring(s) of the carbocyclyl group.

“Heterocarbocycles” or “heterocarbocyclyl” groups are carbocycles which contain from 1 to 4 heteroatoms independently selected from nitrogen, oxygen, and sulphur, wherein the nitrogen and/or sulphur heteroatom(s) may be optionally oxidized, and the nitrogen heteroatom(s) may be optionally quaternized. Heterocarbocycles may be saturated or unsaturated (but not aromatic), monocyclic or polycyclic. Exemplary heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, and the like. The number of “members” of a heterocarbocyclyl group refers to the total number of carbon atoms and heteroatoms in the ring(s) of the heterocarbocyclyl group.

The term “aryl” refers to aromatic homocyclic (i.e., hydrocarbon) mono-, bi- or tricyclic ring-containing groups, preferably having 6 to 12 members, such as phenyl, naphthyl and biphenyl. Optionally, the aryl group is phenyl. In polycyclic aryl groups, the rings can be attached together in a pendant manner or can be fused. The number of “members” of an aryl group refers to the total number of carbon atoms in the ring(s) of the aryl group.

As used herein, “heteroaryl” or “heteroaromatic” refers an aromatic heterocarbocycle having 1 to 4 heteroatoms selected from nitrogen, oxygen and sulfur and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl (pyridinyl), quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl. The number of “members” of a heteroaryl group refers to the total number of carbon atoms and heteroatoms in the ring(s) of the heteroaryl group.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like. In polycyclic heterocyclyl groups, the rings can be attached together in a pendant manner (i.e., two rings are connected by a single bond), in a spiro manner (i.e., two rings are connected through a defining single common atom), in a fused manner (i.e., two rings share two adjacent atoms; in other words, two rings share one covalent bond), in a bridged manner (i.e., two rings share three or more atoms, separating the two bridgehead atoms by a bridge containing at least one atom), or a combination thereof. The number of “members” of a heterocyclyl group refers to the total number of carbon atoms and heteroatoms in the ring(s) of the heterocyclyl group.

“Alkoxy” or “alkyloxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, and t-butoxy.

“Alkylamino” refers an alkyl group as defined above with the indicated number of carbon atoms attached through an amino bridge. An example of an alkylamino is methylamino (i.e., —NH—CH₃).

“Alkylthio” refers to an alkyl group as defined above with the indicated number of carbon atoms attached through a sulfur bridge. An example of an alkylthio is methylthio (i.e., —S—CH₃).

The terms “halogen” and “Hal” refer to fluorine, chlorine, bromine, and iodine.

The term “substituted” refers to a molecule wherein at least one hydrogen atom is replaced with a substituent. The molecule may be multiply substituted. In the case of an oxo substituent (“═O”), two hydrogen atoms are replaced. Example substituents within this context may include halogen, hydroxyl, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{x}R^{y}, —NR^{x}C(═O)R^{y}, —NR^{x}C(═O)NR^{y}R^{z}, —NR^{x}C(═O)OR^{y}, —NR^{x}SO₂R^{y}, —C(═O)R^{x}, —C(═O)OR^{x}, —C(═O)NR^{x}R^{y}, —OC(═O)NR^{x}R^{y}, —OR^{x}, —SR^{x}, —SOR^{x}, —S(═O)₂R^{x}, —OS(═O)₂R^{x}, and —S(═O)₂OR^{x}. R^{x}, R^{y}, and R^{z} in this context may be the same or different, and independently hydrogen, halogen, hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

The term “optionally substituted,” as used herein, means that substitution is optional.

It is understood that any substitution is in accordance with permitted valence of the substituted atom and the substituent, and that the substitution results in a stable compound, i.e., a compound that does not spontaneously undergo transformation such as by rearrangement, cyclization, elimination, etc., at room temperature.

### II. Compounds

Disclosed are negative allosteric modulators of GluN2-containing NMDA receptors. These compounds are highly selective for GluN2C/D over GluN2A/B.

To the extent that chemical formulas described herein contain one or more unspecified chiral centers, the formulas are intended to encompass all stable stereoisomers, enantiomers, and diastereomers. Such compounds can exist as a single enantiomer, a mixture of diastereomers, a racemic mixture, or combinations thereof. It is also understood that the chemical formulas encompass all tautomeric forms.

As used herein, “sulfinyl” refers to —S(═O)R^{B}, wherein R^{B} is an alkyl group, a heteroalkyl group, a carbocyclyl group, a heterocyclyl group, an aryl group, or a heteroaryl group.

As used herein, “sulfonyl” refers to —S(═O)₂R^{C}, wherein R^{C} is an alkyl group, a heteroalkyl group, a carbocyclyl group, a heterocyclyl group, an aryl group, or a heteroaryl group.

As used herein, “pharmaceutically acceptable salt” refers to the modification of the original compound by making the acid or base salts thereof. Examples of pharmaceutically acceptable salts include, but are not limited to, mineral or organic acid salts of basic residues such as amines, as well as alkali or organic salts of acidic residues such as carboxylic acids or phosphorus acids. For original compounds containing a basic residue, the pharmaceutically acceptable salts can be prepared by treating the compounds with an appropriate amount of a non-toxic inorganic or organic acid; alternatively, the pharmaceutically acceptable salts can be formed in situ during preparation of the original compounds. Exemplary salts of the basic residue include salts with an inorganic acid selected from hydrochloric, hydrobromic, sulfuric, sulfamic, phosphoric, and nitric acids or with an organic acid selected from acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic acids. For original compounds containing an acidic residue, the pharmaceutically acceptable salts can be prepared by treating the compounds with an appropriate amount of a non-toxic base; alternatively, the pharmaceutically acceptable salts can be formed in situ during preparation of the original compounds. Exemplary salts of the acidic residue include salts with a base selected from ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, tris(hydroxymethyl)aminomethane, 2-dimethylaminoethanol, 2-diethylaminoethanol, lysine, arginine, and histidine. Optionally, the pharmaceutically acceptable salts can be prepared by reacting the free acid or base form of the original compounds with a stoichiometric amount or more of the appropriate base or acid, respectively, in water, in an organic solvent, or in a mixture thereof. Lists of suitable pharmaceutically acceptable salts can be found in Remington's Pharmaceutical Sciences, 20th Ed., Lippincott Williams & Wilkins, Baltimore, M D, 2000 and Handbook of Pharmaceutical Salts: Properties, Selection, and Use, Stahl and Wermuth, Eds., Wiley-VCH, Weinheim, 2002.

Methods of making exemplary compounds are disclosed in the Examples. The methods are compatible with a wide variety of functional groups and compounds, and thus a wide variety of derivatives can be obtainable from the disclosed methods. Additional synthetic methods can be found in WO2019/191424, WO2014/210456, and WO2010/088408.

### A. Monocyclic-Core Compounds

### 1. General Structure

Generally, the monocyclic-core compounds have a structure of Formula I, an enantiomer or diastereomer thereof, or a pharmaceutically acceptable salt thereof,

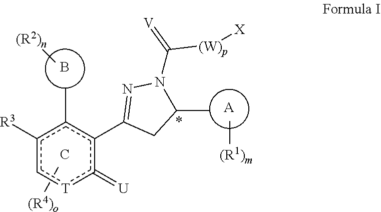

wherein ring A and ring B are independently 5- or 6-membered aryl or heteroaryl;
wherein T is CH, CR⁴, CH₂, CHR⁴, C(R⁴)₂, N, NH, or NR⁴;
wherein U is O, S, or absent,
wherein V is O, S, NH, N-alkyl, two hydrogen atoms each singly bonded to the adjacent carbon, or two fluorine atoms each singly bonded to the adjacent carbon;
wherein W, at each occurrence, is independently CH₂, C(Hal)₂, CH₂CH₂, CH₂CH₂CH₂, C(Hal)₂CH₂, CH₂C(Hal)₂, CH═CH, C(Hal)=CH, CH═C(Hal), C═O, O, S, or NH, optionally substituted by one or more, the same or different R¹⁰;
wherein p is 1, 2, or 3;
wherein X is —C(═Y)Z or 5- or 6-membered nitrogen-containing heterocyclyl optionally substituted by one or more, the same or different R⁶.
wherein Y is O, S, NH, N-alkyl, two hydrogen atoms each singly bonded to the adjacent carbon, or two fluorine atoms each singly bonded to the adjacent carbon;
wherein Z is halogen, OH, O-alkyl, O-alkanoyl, NH₂, NH-alkyl, N(alkyl)₂, NH-alkanoyl, N[(alkyl)(alkanoyl)], SH, S-alkyl, or S-alkanoyl, optionally substituted with one or more, the same or different R¹⁰;
wherein m and n are independently 0, 1, 2, 3, 4, or 5;
wherein o is 0, 1, 2, or 3;
wherein R¹, R², R⁴, and R⁶ are at each occurrence individually and independently alkyl, haloalkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heteroaryl, or heterocyclyl, optionally substituted with one or more, the same or different R¹⁰;
wherein R³ is alkyl or haloalkyl, optionally substituted with one or more, the same or different R¹⁰;
wherein R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl; and
wherein the dotted lines in ring C each independently represent a single bond (i.e., a pair of shared electrons) or no bond (i.e., void).

In some embodiments, T is CH or CR⁴, and U is absent. In some embodiments, T is N and U is absent. In some embodiments, T is NH or NR⁴ and U is O.

In some embodiments, ring A, ring B, or both are independently selected from pyrrolyl (such as 1-pyrrolyl, 2-pyrrolyl, and 3-pyrrolyl), furanyl (such as 2-furanyl and 3-furanyl), thiophenyl (such as 2-thiophenyl and 3-thiophenyl), imidazolyl, pyrazolyl, oxazolyl, isoxazolyl, oxathiolyl, isoxathiolyl, triazolyl, thiazolyl, isothiazolyl, phenyl, pyridinyl (such as 2-pyridinyl, 3-pyridinyl, and 4-pyridinyl), and pyrazinyl. In some embodiments, ring A, ring B, or both are independently selected from:

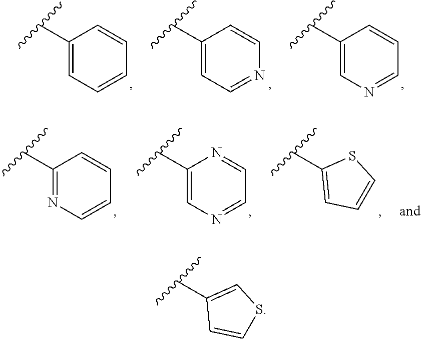

In some embodiments, ring A is 6-membered aryl or heteroaryl such as phenyl. In some embodiments, ring A is 5-membered heteroaryl such as thiophenyl (e.g., 2-thiophenyl and 3-thiophenyl).

In some embodiments, ring B is 6-membered aryl or heteroaryl such as phenyl.

In some embodiments, ring A is 5- or 6-membered aryl or heteroaryl and ring B is 6-membered aryl or heteroaryl. In some embodiments, ring A and ring B are phenyl. In some embodiments, ring A is thiophenyl (such as 2-thiophenyl and 3-thiophenyl) and ring B is phenyl.

In some embodiments, V is O.

In some embodiments, W, at each occurrence, is independently CH₂, C(Hal)₂ (such as CF₂), CH₂CH₂, CH₂CH₂CH₂, C(Hal)₂CH₂ (such as CF₂CH₂), CH₂C(Hal)₂ (such as CH₂CF₂), CH═CH, C(Hal)=CH (such as CF═CH), or CH═C(Hal) (such as CH═CF), optionally substituted by one or more, the same or different R¹⁰. In some embodiments, W, at each occurrence, is independently CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰. In some embodiments, W, at each occurrence, is independently CH₂, CF₂, CH₂CH₂, CF₂CH₂, CH₂CF₂, CH═CH, CF═CH, or CH═CF.

In some embodiments, p is 1 or 2.

In some embodiments, p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰. In some embodiments, p is 1 and W is CH₂, CF₂, CH₂CH₂, CF₂CH₂, CH₂CF₂, CH═CH, CF═CH, or CH═CF.

In some embodiments, X is —C(═Y)Z. In some cases, Y is O. In some cases, Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰. In some embodiments, Y is O and Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰. In some embodiments, X is a carboxylate, ester, or amide. For example, X is —COOH, —C(═O)OEt, —C(═O)OiPr, —C(═O)OiBu, —C(═O)OPh, —C(═O)OBz, —C(═O)NH₂, —C(═O)NHMe, or —C(═O)N(Me)₂.

In some embodiments, X is 5- or 6-membered nitrogen-containing heterocyclyl optionally substituted by one or more, the same or different R⁶. In some cases, X is 5-membered nitrogen-containing heterocyclyl optionally substituted by one or more, the same or different R⁶. In some cases, X is 5-membered nitrogen-containing heteroaryl optionally substituted by one or more, the same or different R⁶.

Optionally, the 5- or 6-membered nitrogen-containing heterocyclyl is selected from tetrazole (such as 1-tetrazole, 2-tetrazole, and 5-tetrazole), imidazole (such as 2-imidazole and 4-imidazole), oxazole (such as 2-oxazole and 4-oxazole), triazole (such as 3-(1,2,4-triazole)), thiazole (such as 2-thiazole and 4-thiazole), thiazolidine dione, oxazolidine dione, oxadiazol-5(4H)-one, thiadiazol-5(4H)-one, oxathiadiazole-2-oxide, and oxadiazol-5(4H)-thione. For example, the 5- or 6-membered nitrogen-containing heterocyclyl is tetrazole.

Optionally, the 5- or 6-membered nitrogen-containing heterocyclyl is selected from:

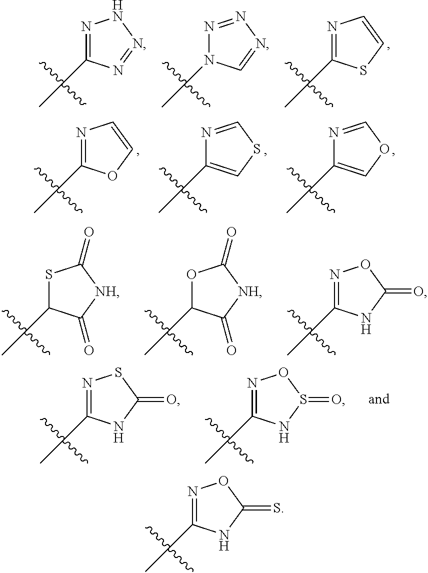

In some embodiments, m is 0, 1, 2, or 3. For example, m is 1. In some embodiments, n is 0, 1, 2, or 3. For example, n is 1. In some embodiments, o is 0 or 1. For example, o is 1.

In some embodiments, one or more of R¹, R², R⁴, and R⁶ are selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, at least one R¹ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰. For example, at least one R¹ is selected from methyl, trifluoromethyl, ethyl, methoxy, ethoxy, halogen, nitro, and cyano. For example, at least one R¹ is halogen, such as chloro.

In some embodiments, at least one R² is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰. For example, at least one R² is selected from methyl, trifluoromethyl, ethyl, methoxy, ethoxy, halogen, nitro, and cyano. For example, at least one R² is halogen, such as chloro.

In some embodiments, at least one R⁴ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

For example, at least one R⁴ is selected from C₁-C₄ alkyl and C₁-C₄ haloalkyl (such as trifluoromethyl). For example, at least one R⁴ is methyl.

In some embodiments, at least one R⁶ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, there is an R¹ at the para position of ring A (m≥1) when ring A is phenyl. For example, there is a halogen group (such as chloro) at the para position of ring A when ring A is phenyl.

In some embodiments, there is an R² at the para position of ring B (n≥1) when ring B is phenyl. For example, there is a halogen group (such as chloro) at the para position of ring B when ring B is phenyl.

In some embodiments, m is 1 and R¹ is halogen. For example, m is 1 and ring A is phenyl substituted with chloro (R¹) at the para position. As another example, m is 1 and ring A is 2-thiophene substituted with chloro (R¹) at the 5 position.

In some embodiments, n is 1 and R² is halogen. For example, n is 1 and ring B is phenyl para-substituted with chloro (R²).

In some embodiments, o is 1 and R⁴ is C₁-C₄ alkyl or C₁-C₄ haloalkyl, such as methyl.

In some embodiments, R³ is C₁-C₄ alkyl or C₁-C₄ haloalkyl. For example, R³ is methyl.

In some embodiments, the compounds have the following features:
ring A and ring B are phenyl;
V is O;
p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰;
X is —C(═O)Z or 5- or 6-membered nitrogen-containing heterocyclyl optionally substituted by one or more, the same or different R⁶;
Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰;
m and n are independently 0, 1, or 2; and
o is 0 or 1.

Optionally, Formula I is in the following configuration:

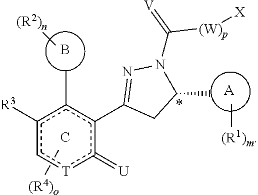

### 2. Exemplary Structures and Compounds

Optionally, the compounds have a structure of Formula IA, an enantiomer or diastereomer thereof, or a pharmaceutically acceptable salt thereof,

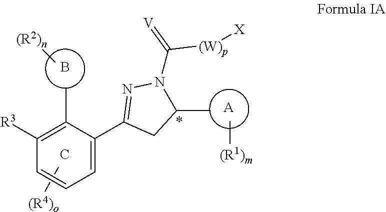

wherein o is 0, 1, 2, or 3; and
wherein ring A, ring B, V, W, X, R¹, R², R³, R⁴, m, n, and p are the same as those described above for Formula I.

Optionally, Formula IA is in the following configuration:

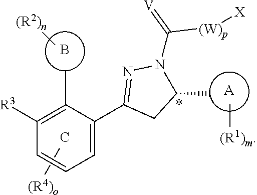

Optionally, the compounds have a structure of Formula IB, an enantiomer or diastereomer thereof, or a pharmaceutically acceptable salt thereof,

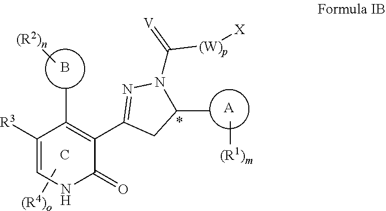

wherein o is 0, 1, or 2; and
wherein ring A, ring B, V, W, X, R¹, R², R³, R⁴, m, n, and p are the same as those described above for Formula I.

Optionally, Formula IB is in the following configuration:

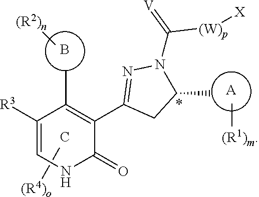

Optionally, the compounds have a structure of Formula IC, an enantiomer or diastereomer thereof, or a pharmaceutically acceptable salt thereof,

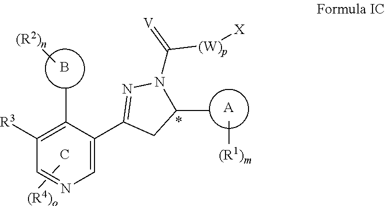

wherein o is 0, 1, or 2; and
wherein ring A, ring B, V, W, X, R¹, R², R³, R⁴, m, n, and p are the same as those described above for Formula I.

Optionally, Formula IC is in the following configuration:

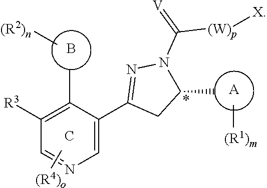

Exemplary compounds of Formulas I, IA, IB, and IC are described in the Examples.

In some embodiments, the compounds are selected from the following and pharmaceutically acceptable salts thereof:

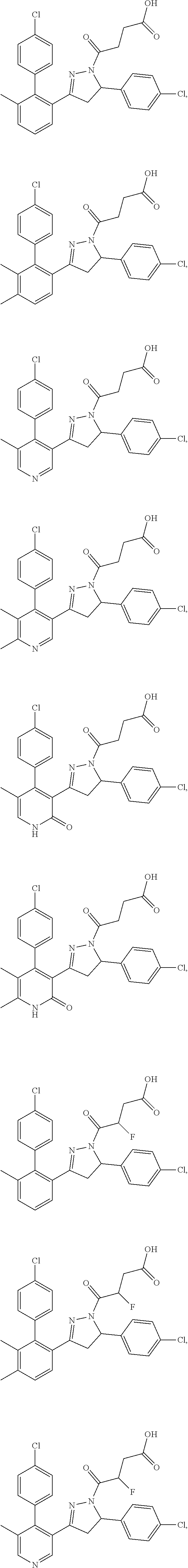

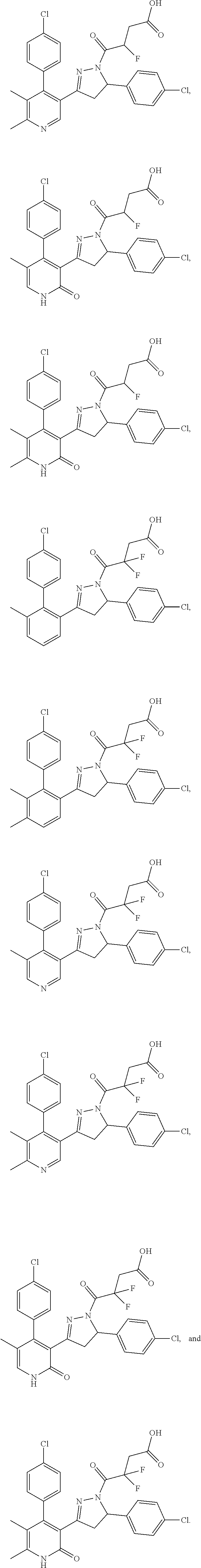

In some embodiments, the compounds are selected from the following and pharmaceutically acceptable salts thereof:

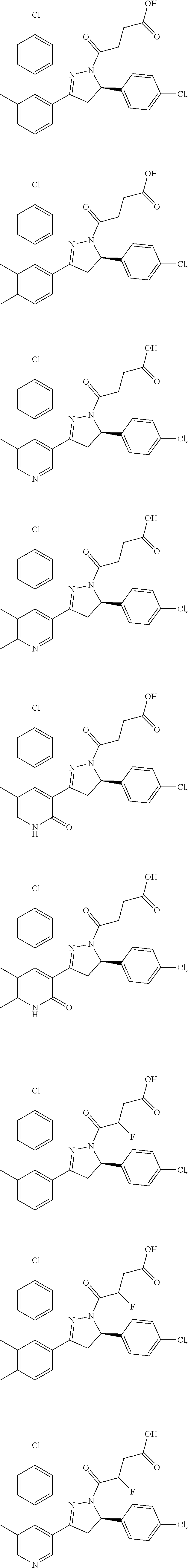

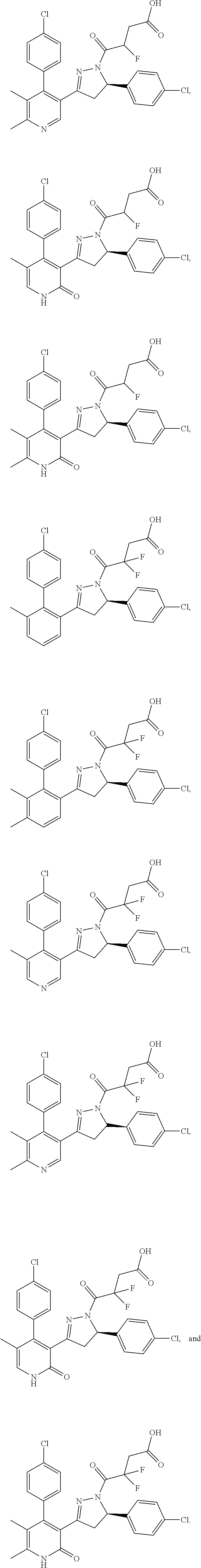

### B. Bicyclic-Core Compounds

### 1. General Structure

Generally, the bicyclic-core compounds have a structure of Formula II, an enantiomer or diastereomer thereof, or a pharmaceutically acceptable salt thereof,

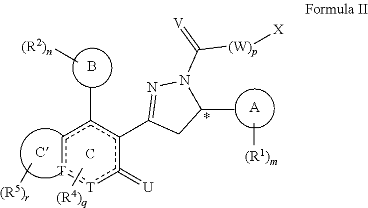

wherein ring A and ring B are independently 5- or 6-membered aryl or heteroaryl;
wherein ring C′ is 5- or 6-membered heteroaryl;
wherein T is CH, CR⁴, CH₂, CHR⁴, C(R⁴)₂, N, NH, or NR⁴;
wherein T′ is C or N;
wherein U is O, S, or absent;
wherein V is O, S, NH, N-alkyl, two hydrogen atoms each singly bonded to the adjacent carbon, or two fluorine atoms each singly bonded to the adjacent carbon;
wherein W, at each occurrence, is independently CH₂, C(Hal)₂, CH₂CH₂, CH₂CH₂CH₂, C(Hal)₂CH₂, CH₂C(Hal)₂, CH═CH, C(Hal)=CH, CH═C(Hal), C═O, O, S, or NH, optionally substituted by one or more, the same or different R¹⁰.
wherein p is 1, 2, or 3;
wherein X is —C(═Y)Z or 5- or 6-membered nitrogen-containing heterocyclyl optionally substituted by one or more, the same or different R⁶;
wherein Y is O, S, NH, N-alkyl, two hydrogen atoms each singly bonded to the adjacent carbon, or two fluorine atoms each singly bonded to the adjacent carbon;
wherein Z is halogen, OH, O-alkyl, O-alkanoyl, NH₂, NH-alkyl, N(alkyl)₂, NH-alkanoyl, N[(alkyl)(alkanoyl)], SH, S-alkyl, or S-alkanoyl, optionally substituted with one or more, the same or different R¹⁰;
wherein m and n are independently 0, 1, 2, 3, 4, or 5;
wherein q is 0, 1, or 2;
wherein r is 0, 1, 2, or 3;
wherein R¹, R², R⁴, R⁵, and R⁶, at each occurrence, are individually and independently alkyl, haloalkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heteroaryl, or heterocyclyl, optionally substituted with one or more, the same or different R¹⁰;
wherein R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl; and
wherein the dotted lines in ring C each independently represent a single bond (i.e., a pair of shared electrons) or no bond (i.e., void).

In some embodiments, ring C′ is 5-membered heteroaryl, such as thiophene, pyrrole, pyrazole, oxathiole, isoxathiole, thiazole, or isothiazole. For example, ring C′ is thiophene.

In some embodiments, ring C′ is 6-membered heteroaryl, such as pyridine or diazine. For example, ring C′ is pyridine.

In some embodiments, T is CH or CR⁴, and U is absent. In some embodiments, T is N and U is absent. In some embodiments, T is NH or NR⁴ and U is 0.

In some embodiments, T′ is C. In some embodiments, T′ is N.

In some embodiments, T is NH or NR⁴, T′ is C, and U is O.

In some embodiments, ring A, ring B, or both are independently selected from pyrrolyl (such as 1-pyrrolyl, 2-pyrrolyl, and 3-pyrrolyl), furanyl (such as 2-furanyl and 3-furanyl), thiophenyl (such as 2-thiophenyl and 3-thiophenyl), imidazolyl, pyrazolyl, oxazolyl, isoxazolyl, oxathiolyl, isoxathiolyl, triazolyl, thiazolyl, isothiazolyl, phenyl, pyridinyl (such as 2-pyridinyl, 3-pyridinyl, and 4-pyridinyl), and pyrazinyl. In some embodiments, ring A, ring B, or both are independently selected from:

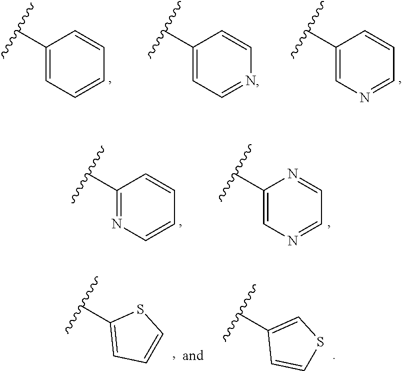

In some embodiments, ring A is 6-membered aryl or heteroaryl such as phenyl. In some embodiments, ring A is 5-membered heteroaryl such as thiophenyl (e.g., 2-thiophenyl and 3-thiophenyl).

In some embodiments, ring B is 6-membered aryl or heteroaryl such as phenyl.

In some embodiments, ring A is 5- or 6-membered aryl or heteroaryl and ring B is 6-membered aryl or heteroaryl. In some embodiments, ring A and ring B are phenyl. In some embodiments, ring A is thiophenyl (such as 2-thiophenyl and 3-thiophenyl) and ring B is phenyl.

In some embodiments, V is O.

In some embodiments, W, at each occurrence, is independently CH₂, C(Hal)₂ (such as CF₂), CH₂CH₂, CH₂CH₂CH₂, C(Hal)₂CH₂ (such as CF₂CH₂), CH₂C(Hal)₂ (such as CH₂CF₂), CH═CH, C(Hal)=CH (such as CF═CH), or CH═C(Hal) (such as CH═CF), optionally substituted by one or more, the same or different R¹⁰. In some embodiments, W, at each occurrence, is independently CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰. In some embodiments, W, at each occurrence, is independently CH₂, CF₂, CH₂CH₂, CF₂CH₂, CH₂CF₂, CH═CH, CF═CH, or CH═CF.

In some embodiments, p is 1 or 2.

In some embodiments, p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰. In some embodiments, p is 1 and W is CH₂, CF₂, CH₂CH₂, CF₂CH₂, CH₂CF₂, CH═CH, CF═CH, or CH═CF.

In some embodiments, X is —C(═Y)Z. In some cases, Y is O. In some cases, Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰. In some embodiments, Y is O and Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰. In some embodiments, X is a carboxylate, ester, or amide. For example, X is —COOH, —C(═O)OEt, —C(═O)OiPr, —C(═O)OiBu, —C(═O)OPh, —C(═O)OBz, —C(═O)NH₂, —C(═O)NHMe, or —C(═O)N(Me)₂.

In some embodiments, X is 5- or 6-membered nitrogen-containing heterocyclyl optionally substituted by one or more, the same or different R⁹. In some cases, X is 5-membered nitrogen-containing heterocyclyl optionally substituted by one or more, the same or different R⁶. In some cases, X is 5-membered nitrogen-containing heteroaryl optionally substituted by one or more, the same or different R⁶.

Optionally, the 5- or 6-membered nitrogen-containing heterocyclyl is selected from tetrazole (such as 1-tetrazole, 2-tetrazole, and 5-tetrazole), imidazole (such as 2-imidazole and 4-imidazole), oxazole (such as 2-oxazole and 4-oxazole), triazole (such as 3-(1,2,4-triazole)), thiazole (such as 2-thiazole and 4-thiazole), thiazolidine dione, oxazolidine dione, oxadiazol-5(4H)-one, thiadiazol-5(4H)-one, oxathiadiazole-2-oxide, and oxadiazol-5(4H)-thione. For example, the 5- or 6-membered nitrogen-containing heterocyclyl is tetrazole.

Optionally, the 5- or 6-membered nitrogen-containing heterocyclyl is selected from:

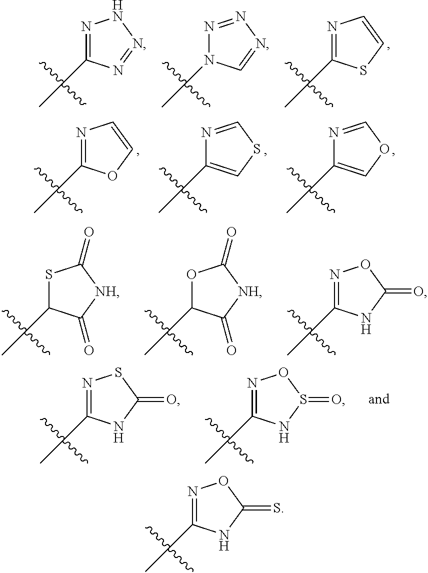

In some embodiments, m is 0, 1, 2, or 3. For example, m is 1. In some embodiments, n is 0, 1, 2, or 3. For example, n is 1. In some embodiments, q is 0 or 1. For example, q is 0. In some embodiments, r is 0, 1, or 2. For example, r is 0.

In some embodiments, one or more of R¹, R², R⁴, R⁵, and R⁶ are selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, at least one R¹ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰. For example, at least one R¹ is selected from methyl, trifluoromethyl, ethyl, methoxy, ethoxy, halogen, nitro, and cyano. For example, at least one R¹ is halogen, such as chloro.

In some embodiments, at least one R² is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰. For example, at least one R² is selected from methyl, trifluoromethyl, ethyl, methoxy, ethoxy, halogen, nitro, and cyano. For example, at least one R² is halogen, such as chloro.

In some embodiments, at least one R⁴ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, at least one R⁵ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, at least one R⁶ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, there is an R¹ at the para position of ring A (m≥1) when ring A is phenyl. For example, there is a halogen group (such as chloro) at the para position of ring A when ring A is phenyl.

In some embodiments, there is an R² at the para position of ring B (n≥1) when ring B is phenyl. For example, there is a halogen group (such as chloro) at the para position of ring B when ring B is phenyl.

In some embodiments, m is 1 and R¹ is halogen. For example, m is 1 and ring A is phenyl substituted with chloro (R¹) at the para position. As another example, m is 1 and ring A is 2-thiophene substituted with chloro (R¹) at the 5 position.

In some embodiments, n is 1 and R² is halogen. For example, n is 1 and ring B is phenyl para-substituted with chloro (R²).

In some embodiments, the compounds have the following features:
ring A and ring B are phenyl;
V is O;
p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰;
X is —C(═O)Z or 5- or 6-membered nitrogen-containing heterocyclyl optionally substituted by one or more, the same or different R⁶;
Z is OH, O-alkyl, O-alkanoyl, NH₂, NH-alkyl, N(alkyl)₂, NH-alkanoyl, or N[(alkyl)(alkanoyl)], optionally substituted with one or more, the same or different R¹⁰;
m and n are independently 0, 1, or 2;
q is 0 or 1; and
r is 0, 1, or 2.

Optionally, Formula II is in the following configuration:

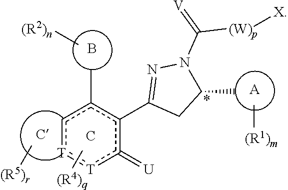

### 2. Exemplary Structures and Compounds

Optionally, the compounds have a structure of Formula IIA, an enantiomer or diastereomer thereof, or a pharmaceutically acceptable salt thereof,

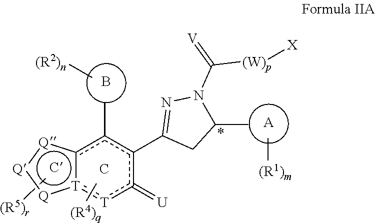

wherein Q, Q′, and Q″ are independently selected from S, O, N, NH, NR⁵, CH, or CR⁵;
wherein at least one of Q, Q′, and Q″ contains a heteroatom;
wherein r is 0, 1, or 2; and
wherein ring A, ring B, T, T′, U, V, W, X, R¹, R², R⁴, R⁵, m, n, p, and q are the same as those described above in Formula II.

In some embodiments, Q is S. In some embodiments, Q is O. In some embodiments, Q is N, NH, or NR⁵. In some embodiments, Q is CH or CR⁵. In some embodiments, Q′ is CH or CR⁵. In some embodiments, Q″ is CH or CR⁵. In some embodiments, Q is S, Q′ is CH or CR⁵, and Q″ is CH or CR⁵.

It is understood that the value of r in Formula IIA represents the number of incidences in which any of Q, Q′, and Q″ is NR⁵ or CR⁵.

Optionally, Formula IIA is in the following configuration:

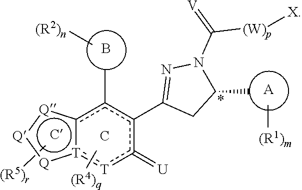

Exemplary compounds of Formulas II and IIA are described in the Examples.

In some embodiments, the compounds are selected from the following and pharmaceutically acceptable salts thereof:

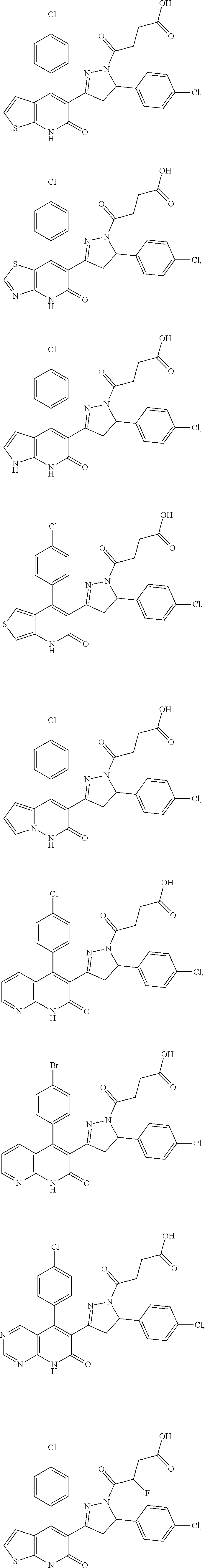

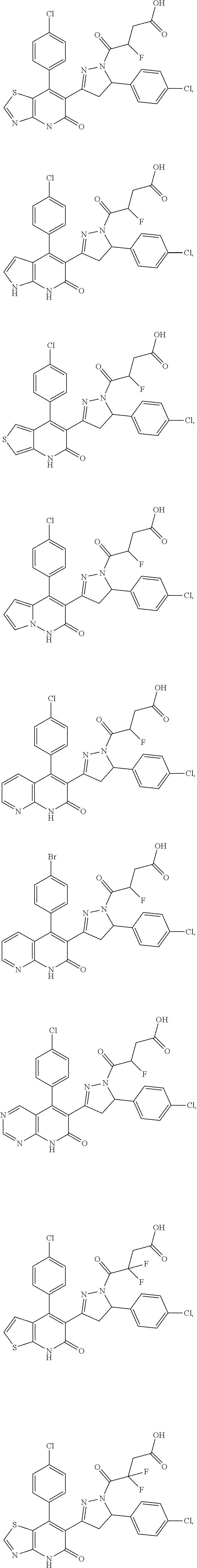

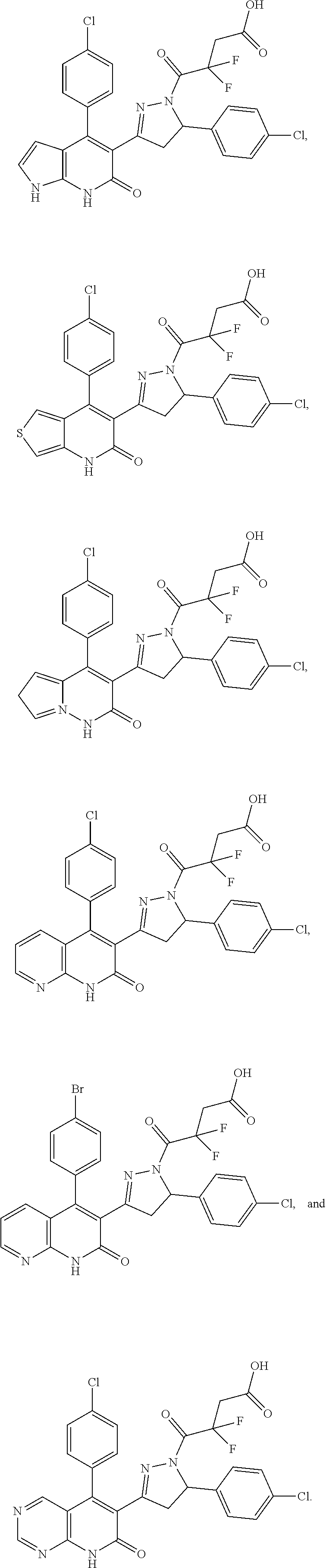

In some embodiments, the compounds are selected from the following and pharmaceutically acceptable salts thereof:

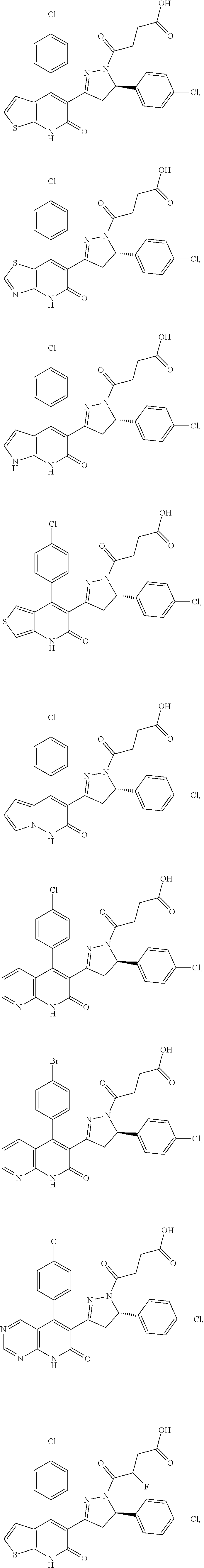

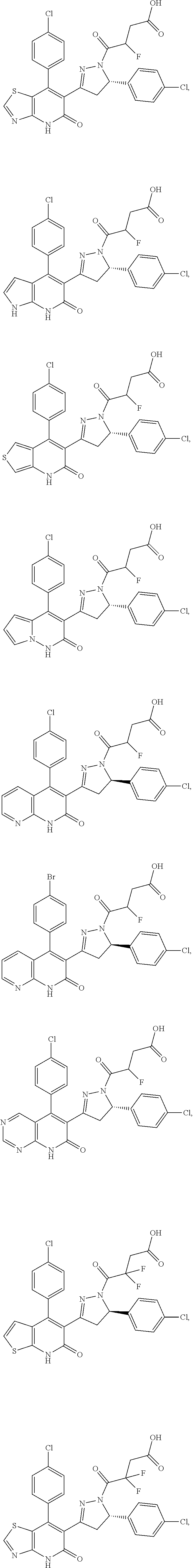

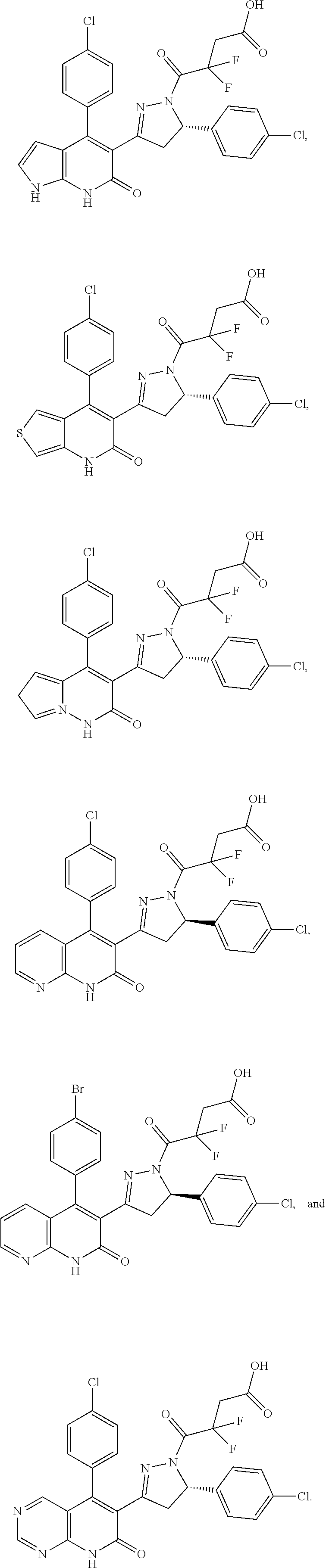

### C. Additional Compounds

### 1. General Structure

Generally, these additional compounds have a structure of Formula III, an enantiomer or diastereomer thereof, or a pharmaceutically acceptable salt thereof,

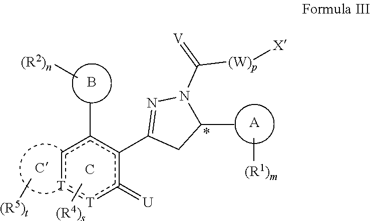

wherein ring A and ring B are independently 5- or 6-membered aryl or heteroaryl;
wherein ring C′ is 5- or 6-membered aryl or heterocyclyl or absent;
wherein T is CH, CR⁴, CH₂, CHR⁴, C(R⁴)₂, N, NH, or NR⁴;
wherein T′ is C or N when ring C′ is present and is CH, CR⁴, CH₂, CHR⁴, C(R⁴)₂, N, NH, or NR⁴ when ring C′ is absent;
wherein U is O, S, or absent;
wherein V is O, S, NH, N-alkyl, two hydrogen atoms each singly bonded to the adjacent carbon, or two fluorine atoms each singly bonded to the adjacent carbon;
wherein W, at each occurrence, is independently CH₂, C(Hal)₂, CH₂CH₂, CH₂CH₂CH₂, C(Hal)₂CH₂, CH₂C(Hal)₂, CH═CH, C(Hal)=CH, CH═C(Hal), C═O, O, S, or NH, optionally substituted by one or more, the same or different R¹⁰;
wherein p is 1, 2, or 3;
wherein X′ is 5- or 6-membered nitrogen-containing heterocyclyl optionally substituted by one or more, the same or different R⁶;
wherein m and n are independently 0, 1, 2, 3, 4, or 5;
wherein s is 0, 1, 2, 3, or 4 when ring C′ is absent and is 0, 1, or 2 when ring C′ is present;
wherein t is 0 when ring C′ is absent and is 0, 1, 2, 3, or 4 when ring C′ is present;
wherein R¹, R², R⁴, R⁵, and R⁶, at each occurrence, are individually and independently alkyl, haloalkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heteroaryl, or heterocyclyl, optionally substituted with one or more, the same or different R¹⁰;
wherein R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl; and
wherein the dotted lines in ring C each independently represent a single bond (i.e., a pair of shared electrons) or no bond (i.e., void).

In some embodiments, ring C′ is present. In some embodiments, ring C′ is 5-membered heteroaryl, such as thiophene, pyrrole, pyrazole, oxathiole, isoxathiole, thiazole, and isothiazole. For example, ring C′ is thiophene. In some embodiments, ring C′ is 6-membered aryl or heteroaryl, such as phenyl, pyridine, and diazine. For example, ring C′ is phenyl. In some embodiments, T is CH or CR⁴, and U is absent. In some embodiments, T is N and U is absent. In some embodiments, T is NH or NR⁴ and U is O. In some embodiments, T′ is C. In some embodiments, T′ is N. In some embodiments, T is NH or NR⁴, T′ is C, and U is O.

In some embodiments, ring C′ is absent. In some embodiments, T is CH or CR⁴, and U is absent. In some embodiments, T is N and U is absent. In some embodiments, T is NH or NR⁴ and U is O. In some embodiments, T′ is CH or CR⁴. In some embodiments, T is CH or CR⁴, T′ is CH or CR⁴, and U is absent. In some embodiments, T is NH or NR⁴, T′ is CH or CR⁴, and U is O. In some embodiments, T is N, T′ is CH or CR⁴, and U is absent.

In some embodiments, ring A, ring B, or both are independently selected from pyrrolyl (such as 1-pyrrolyl, 2-pyrrolyl, and 3-pyrrolyl), furanyl (such as 2-furanyl and 3-furanyl), thiophenyl (such as 2-thiophenyl and 3-thiophenyl), imidazolyl, pyrazolyl, oxazolyl, isoxazolyl, oxathiolyl, isoxathiolyl, triazolyl, thiazolyl, isothiazolyl, phenyl, pyridinyl (such as 2-pyridinyl, 3-pyridinyl, and 4-pyridinyl), and pyrazinyl. In some embodiments, ring A, ring B, or both are independently selected from:

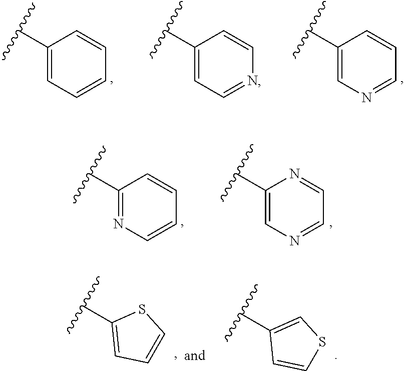

In some embodiments, ring A is 6-membered aryl or heteroaryl such as phenyl. In some embodiments, ring A is 5-membered heteroaryl such as thiophenyl (e.g., 2-thiophenyl and 3-thiophenyl).

In some embodiments, ring B is 6-membered aryl or heteroaryl such as phenyl.

In some embodiments, ring A is 5- or 6-membered aryl or heteroaryl and ring B is 6-membered aryl or heteroaryl. In some embodiments, ring A and ring B are phenyl. In some embodiments, ring A is thiophenyl (such as 2-thiophenyl and 3-thiophenyl) and ring B is phenyl.

In some embodiments, V is O.

In some embodiments, W, at each occurrence, is independently CH₂, C(Hal)₂ (such as CF₂), CH₂CH₂, CH₂CH₂CH₂, C(Hal)₂CH₂ (such as CF₂CH₂), CH₂C(Hal)₂ (such as CH₂CF₂), CH═CH, C(Hal)=CH (such as CF═CH), or CH═C(Hal) (such as CH═CF), optionally substituted by one or more, the same or different R¹⁰. In some embodiments, W, at each occurrence, is independently CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰. In some embodiments, W, at each occurrence, is independently CH₂, CF₂, CH₂CH₂, CF₂CH₂, CH₂CF₂, CH═CH, CF═CH, or CH═CF.

In some embodiments, p is 1 or 2.

In some embodiments, p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰. In some embodiments, p is 1 and W is CH₂, CF₂, CH₂CH₂, CF₂CH₂, CH₂CF₂, CH═CH, CF═CH, or CH═CF.

In some embodiments, m is 0, 1, 2, or 3. For example, m is 1. In some embodiments, n is 0, 1, 2, or 3. For example, n is 1.

In some embodiments, when ring C′ is absent, t is 0 and s is 0, 1, 2, or 3. In some embodiments, when ring C′ is absent, t is 0 and s is 0, 1, or 2. For example, when ring C′ is absent, t is 0 and s is 1. For example, when ring C′ is absent, t is 0 and s is 2.

In some embodiments, when ring C′ is present, t is 0, 1, 2, or 3 and s is 0 or 1. In some embodiments, when ring C′ is present, s is 0 or 1. For example, when ring C′ is present, s is 0. In some embodiments, when ring C′ is present, t is 0, 1, or 2. For example, when ring C′ is present, t is 0.

In some embodiments, X′ is 5-membered nitrogen-containing heterocyclyl optionally substituted by one or more, the same or different R⁶. For example, X′ is 5-membered nitrogen-containing heteroaryl optionally substituted by one or more, the same or different R⁶.

Optionally, the 5- or 6-membered nitrogen-containing heterocyclyl is selected from tetrazole (such as 1-tetrazole, 2-tetrazole, and 5-tetrazole), imidazole (such as 2-imidazole and 4-imidazole), oxazole (such as 2-oxazole and 4-oxazole), triazole (such as 3-(1,2,4-triazole)), thiazole (such as 2-thiazole and 4-thiazole), thiazolidine dione, oxazolidine dione, oxadiazol-5(4H)-one, thiadiazol-5(4H)-one, oxathiadiazole-2-oxide, and oxadiazol-5(4H)-thione. For example, the 5- or 6-membered nitrogen-containing heterocyclyl is tetrazole.

Optionally, the 5- or 6-membered nitrogen-containing heterocyclyl is selected from:

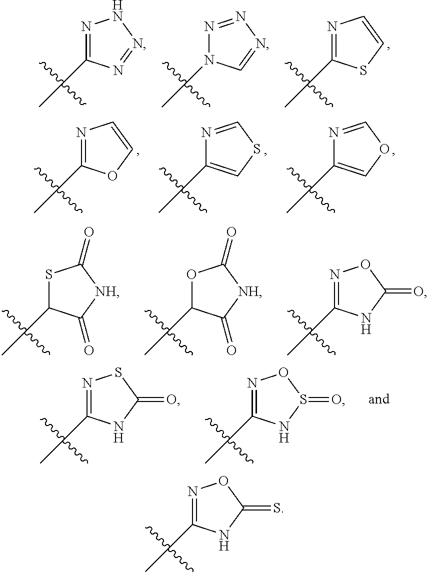

In some embodiments, one or more of R¹, R², R⁴, R⁵ and R⁶ are selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, at least one R¹ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

For example, at least one R¹ is selected from methyl, trifluoromethyl, ethyl, methoxy, ethoxy, halogen, nitro, and cyano. For example, at least one R¹ is halogen, such as chloro.

In some embodiments, at least one R² is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

For example, at least one R² is selected from methyl, trifluoromethyl, ethyl, methoxy, ethoxy, halogen, nitro, and cyano. For example, at least one R² is halogen, such as chloro.

In some embodiments, at least one R⁴ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, at least one R⁵ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰.

In some embodiments, at least one R⁶ is selected from C₁-C₄ alkyl, C₁-C₄ haloalkyl (such as trifluoromethyl), halogen, nitro, cyano, C₁-C₄ alkoxy, C₁-C₄ alkanoyl, carbocyclyl, aryl, heteroaryl, and heterocyclyl, optionally substituted with one or more, the same or different R¹⁰ In some embodiments, there is an R¹ at the para position of ring A (m≥1) when ring A is phenyl. For example, there is a halogen group (such as chloro) at the para position of ring A when ring A is phenyl.

In some embodiments, there is an B² at the para position of ring B (n≥1) when ring B is phenyl. For example, there is a halogen group (such as chloro) at the para position of ring B when ring B is phenyl.

In some embodiments, m is 1 and R¹ is halogen. For example, m is 1 and ring A is phenyl substituted with chloro (R¹) at the para position. As another example, m is 1 and ring A is 2-thiophene substituted with chloro (R¹) at the 5 position.

In some embodiments, n is 1 and R² is halogen. For example, n is 1 and ring B is phenyl para-substituted with chloro (R²).

In some embodiments, when ring C′ is absent, t is 0 and s is 1 or 2, wherein at least one of R⁴ is C₁-C₄ alkyl or C₁-C₄ haloalkyl, optionally substituted with one or more, the same or different R¹⁰. In some embodiments, when ring C′ is absent, t is 0 and s is 2, wherein both R⁴ groups are independently C₁-C₄ alkyl or C₁-C₄ haloalkyl. For example, when ring C′ is absent, t is 0 and s is 2, wherein both R⁴ groups are methyl.

In some embodiments, the compounds have the following features:
ring A and ring B are phenyl;
V is O;
p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰; and
m and n are independently 0, 1, or 2.

Optionally, Formula III is in the following configuration:

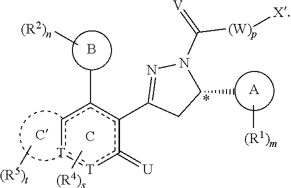

### 2. Exemplary Structures and Compounds

Optionally, the compounds have a structure of Formula IIIA, an enantiomer or diastereomer thereof, or a pharmaceutically acceptable salt thereof,

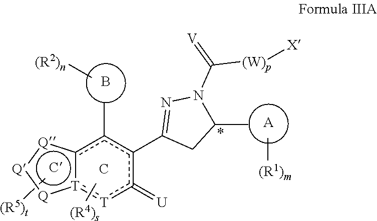

wherein Q, Q′, and Q″ are independently selected from S, O, N, NH, NR⁵, CH, and CR⁵;
wherein at least one of Q, Q′, and Q″ contains a heteroatom;
wherein t is 0, 1, 2, or 3;
wherein s is 0, 1, or 2; and
wherein ring A, ring B, T, T′, U, V, W, X′, R¹, R², R⁴, R⁵, m, n, and p are the same as those described above in Formula III.

It is understood that the value of t in Formula IIIA represents the number of incidences in which any of Q, Q′, and Q″ is NR⁵ or CR⁵.

In some embodiments, Q is S. In some embodiments, Q is 0. In some embodiments, Q is N, NH, or NR⁵. In some embodiments, Q is CH or CR⁵. In some embodiments, Q′ is CH or CR⁵.

In some embodiments, Q″ is CH or CR⁵. In some embodiments, Q is S, Q′ is CH or CR⁵, and Q″ is CH or CR⁵.

Optionally, Formula IIIA is in the following configuration:

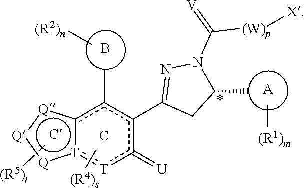

Optionally, the compounds have a structure of Formula IIIB, an enantiomer or diastereomer thereof, or a pharmaceutically acceptable salt thereof,

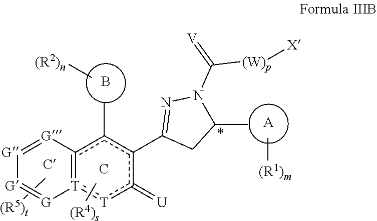

wherein G, G′, G″, and G′″ are independently CH, CR⁵ or N;
wherein t is 0, 1, 2, 3, or 4;
wherein s is 0, 1, or 2; and
wherein ring A, ring B, T, T′, U, V, W, X′, R¹, R², R⁴, R⁵, m, n, and p are the same as those described above in Formula III.

It is understood that the value of t in Formula IIIB represents the number of incidences in which any of G, G′, G″, and G′″ is CR⁵.

In some embodiments, G, G′, G″, and G′″ are independently CH or CR⁵. For example, G, G′, G″, and G′″ are CH (t is 0).

Optionally, Formula IIIB is in the following configuration:

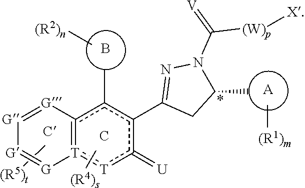

Optionally, the compounds have a structure of Formula IIIC, an enantiomer or diastereomer thereof, or a pharmaceutically acceptable salt thereof,

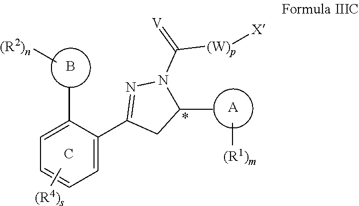

wherein s is 0, 1, 2, 3, or 4; and
wherein ring A, ring B, V, W, X′, R¹, R², R⁴, m, n, and p are the same as those described above for Formula III.

Optionally, Formula IIIC is in the following configuration:

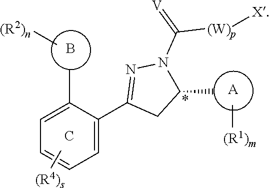

In some embodiments, Formula IIIC is Formula IIIC′, as shown below:

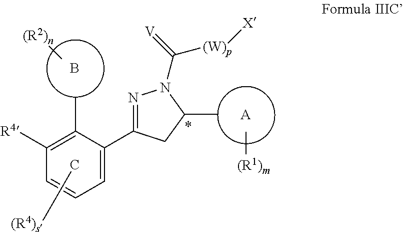

wherein R^{4′} has the same scope as R⁴; and
wherein s′ is 0, 1, 2, or 3.

In some embodiments, R^{4′} is alkyl or haloalkyl, optionally substituted with one or more, the same or different R¹⁰. In some embodiments, R^{4′} is C₁-C₄ alkyl or C₁-C₄ haloalkyl, optionally substituted with one or more, the same or different R¹⁰. In some embodiments, R^{4′} is C₁-C₄ alkyl or C₁-C₄ haloalkyl, such as methyl.

In some embodiments, s′ is 1 and R⁴ is C₁-C₄ alkyl or C₁-C₄ haloalkyl, such as methyl.

Optionally, Formula IIIC′ has the following configuration:

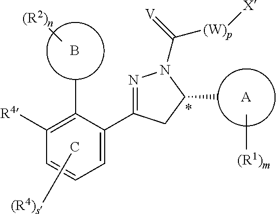

Optionally, the compounds have a structure of Formula HID, an enantiomer or diastereomer thereof, or a pharmaceutically acceptable salt thereof,

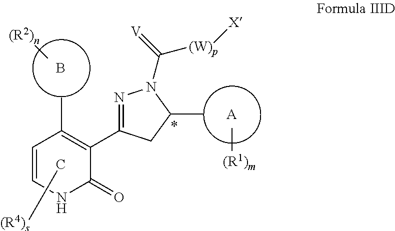

wherein s is 0, 1, 2, or 3; and
wherein ring A, ring B, V, W, X′, R¹, R², R⁴, m, n, and p are the same as those described above in Formula III.

Optionally, Formula IIID is in the following configuration:

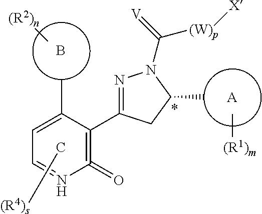

In some embodiments, Formula HID is Formula IIID′, as shown below:

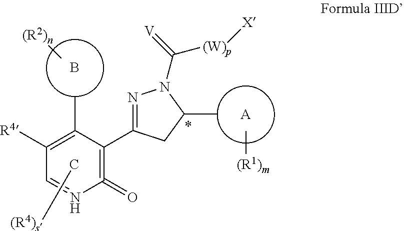

wherein R^{4′} has the same scope as R⁴; and
wherein s′ is 0, 1, or 2.

In some embodiments, R^{4′} is alkyl or haloalkyl, optionally substituted with one or more, the same or different R¹⁰. In some embodiments, R^{4′} is C₁-C₄ alkyl or C₁-C₄ haloalkyl, optionally substituted with one or more, the same or different R¹⁰. In some embodiments, R^{4′} is C₁-C₄ alkyl or C₁-C₄ haloalkyl, such as methyl.

In some embodiments, s′ is 1 and R⁴ is C₁-C₄ alkyl or C₁-C₄ haloalkyl, such as methyl.

Optionally, Formula IIID′ has the following configuration:

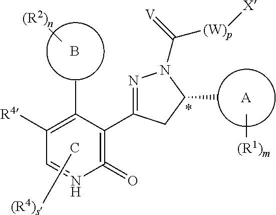

Optionally, the compounds have a structure of Formula IIIE, an enantiomer or diastereomer thereof, or a pharmaceutically acceptable salt thereof,

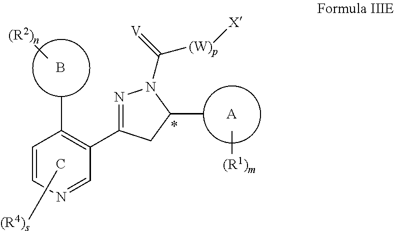

wherein s is 0, 1, 2, or 3; and
wherein ring A, ring B, V, W, X′, R¹, R², R⁴, m, n, and p are the same as those described above in Formula III.

Optionally, Formula IIIE is in the following configuration:

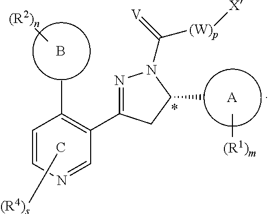

In some embodiments, Formula IIIE is Formula IIIE′, as shown below:

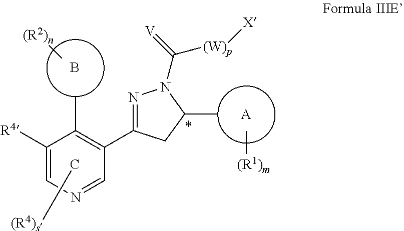

wherein R^{4′} has the same scope as R⁴; and
wherein s′ is 0, 1, or 2.

In some embodiments, R^{4′} is alkyl or haloalkyl, optionally substituted with one or more, the same or different R¹⁰. In some embodiments, R^{4′} is C₁-C₄ alkyl or C₁-C₄ haloalkyl, optionally substituted with one or more, the same or different R¹⁰. In some embodiments, R^{4′} is C₁-C₄ alkyl or C₁-C₄ haloalkyl, such as methyl.

In some embodiments, s′ is 1 and R⁴ is C₁-C₄ alkyl or C₁-C₄ haloalkyl, such as methyl.

Optionally, Formula IIIE′ has the following configuration:

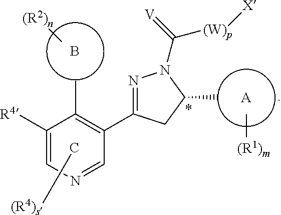

Optionally, the compounds have a structure of Formula IIIF, an enantiomer or diastereomer thereof, or a pharmaceutically acceptable salt thereof,

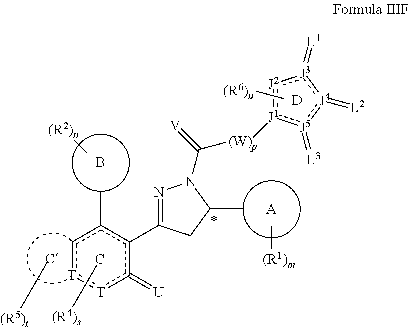

wherein u is 0, 1, 2, 3, or 4;
wherein J¹, J², J³, J⁴, and J⁵ are independently selected from S, O, N, NH, NR⁶, C, CH, and CR⁶;
wherein at least one of J¹, J³, J⁴, and J⁵ is N, NH, or NR⁶ and at least one of J¹, J², J³, J⁴ and J⁵ is C, CH, or CR⁶;
wherein L¹, L², and L³ are independently O, S, or absent;
wherein when L¹ is not absent, J³ is C or S;
wherein when L² is not absent, J⁴ is C or S;
wherein when L³ is not absent, J⁵ is C or S; and
wherein the dotted lines in ring D each independently represent a single bond (i.e., a pair of shared electrons) or no bond (i.e., void).

It is understood that the value of u in Formula IIIF represents the number of incidences in which any of J, J², J³, J⁴, and J⁵ is NR⁶ or CR⁶.

In some embodiments, u is 0, 1, or 2. For example, u is 0.

In some embodiments, L¹, L², and L³ are absent. In some embodiments, L¹ and L³ are absent and L² is O or S. In some embodiments, L¹ and L³ are independently O or S and L² is absent.

In some embodiments, ring D is selected from tetrazole (such as 1-tetrazole, 2-tetrazole, and 5-tetrazole), imidazole (such as 2-imidazole and 4-imidazole), oxazole (such as 2-oxazole and 4-oxazole), triazole (such as 3-(1,2,4-triazole)), thiazole (such as 2-thiazole and 4-thiazole), thiazolidine dione, oxazolidine dione, oxadiazol-5(4H)-one, thiadiazol-5(4H)-one, oxathiadiazole-2-oxide, and oxadiazol-5(4H)-thione. For example, ring D is tetrazole.

Optionally, ring D is selected from:

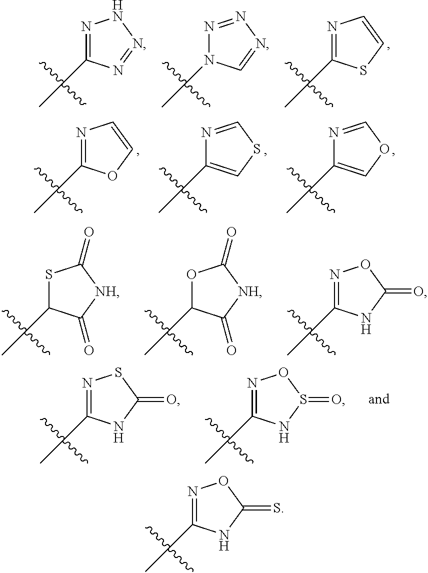

Optionally, Formula IIIF has the following configuration:

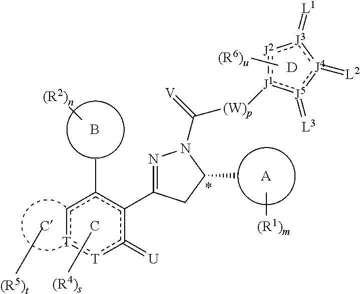

Optionally, the compounds have a structure of Formula IIIFA, an enantiomer or diastereomer thereof, or a pharmaceutically acceptable salt thereof,

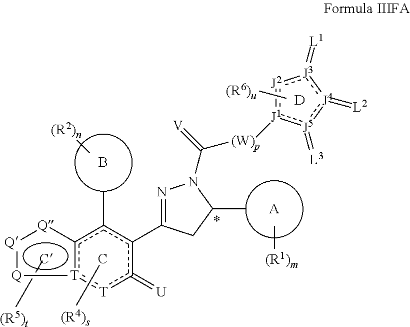

wherein Q, Q′, and Q″ are independently selected from S, O, N, NH, NR⁵, CH, and CR⁵;
wherein at least one of Q, Q′, and Q″ contains a heteroatom;
wherein t is 0, 1, 2, or 3;
wherein s is 0, 1, or 2; and
wherein ring A, ring B, ring D, T, T′, U, V W, R¹, R², R⁴, R⁵, R⁶, J¹, J², J³, J⁴, J⁵, L¹, L², L³, m, n, p, and u are the same as those described above in Formula IIIF.

It is understood that the value of t in Formula IIIFA represents the number of incidences in which any of Q, Q′, and Q″ is NR⁵ or CR⁵.

In some embodiments, Q is S. In some embodiments, Q is O. In some embodiments, Q is N, NH, or NR. In some embodiments, Q is CH or CR⁵. In some embodiments, Q′ is CH or CR⁵.

In some embodiments, Q″ is CH or CR⁵. In some embodiments, Q is S, Q′ is CH or CR⁵, and Q″ is CH or CR⁵.

Optionally, Formula IIIFA is in the following configuration:

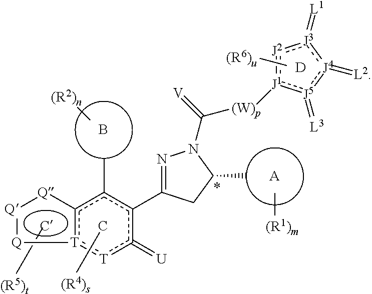

Optionally, the compounds have a structure of Formula IIIFB, an enantiomer or diastereomer thereof, or a pharmaceutically acceptable salt thereof,

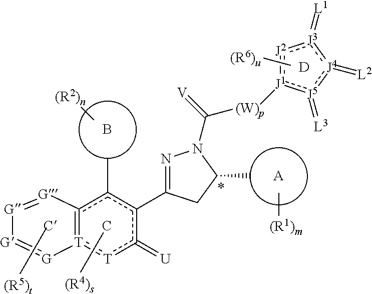

wherein G, G′, G″, and G′″ are independently CH, CR⁵ or N;
wherein t is 0, 1, 2, 3, or 4;
wherein s is 0, 1, or 2; and
wherein ring A, ring B, ring D, T, T′, U, V W, R¹, R², R⁴, R⁵, R⁶, J¹, J², J³, J⁴, J⁵, L¹, L², L³, m, n, p, and u are the same as those described above in Formula IIIF.

It is understood that the value of t in Formula IIIFB represents the number of incidences in which any of G, G′, G″, and G′″ is CR⁵.

In some embodiments, G, G′, G″, and G′″ are independently CH or CR⁵. For example, G, G′, G″, and G′″ are CH (t is 0).

Optionally, Formula IIIFB is in the following configuration:

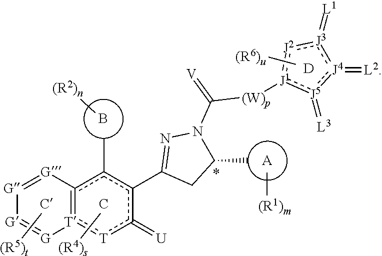

In some embodiments, Formula IIIFB is Formula IIIFB′, as shown below:

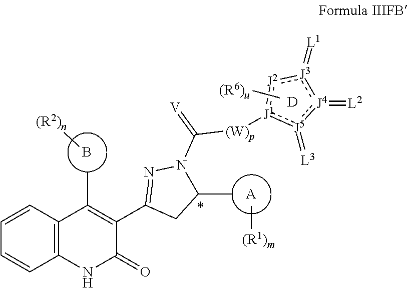

Optionally, Formula IIIFB′ is in the following configuration:

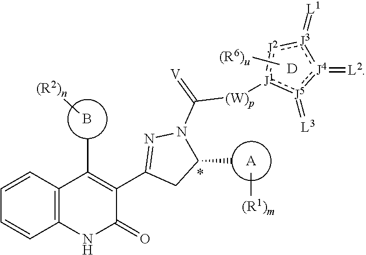

Optionally, the compounds have a structure of Formula IIIFC, an enantiomer or diastereomer thereof, or a pharmaceutically acceptable salt thereof.

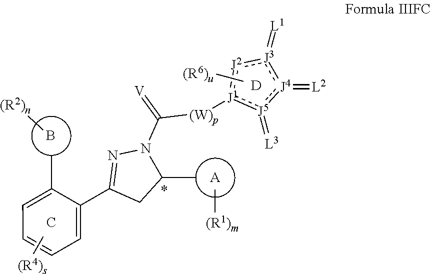

wherein s is 0, 1, 2, 3, or 4; and
wherein ring A, ring B, ring D, T, T′, U, V, W, R¹, R², R⁴, R⁵, R⁶, J¹, J², J³, J⁴, J⁵, L¹, L², L³, m, n, p, and u are the same as those described above in Formula IIIF.

Optionally, Formula IIIFC is in the following configuration:

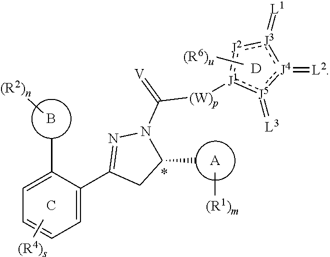

In some embodiments, Formula IIIFC is Formula IIIFC′, as shown below:

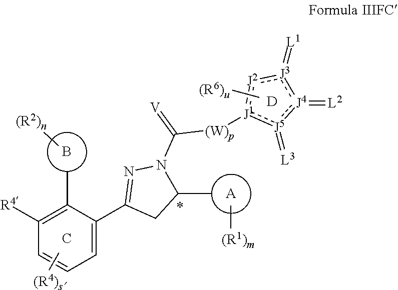

wherein R^{4′} has the same scope as R⁴; and
wherein s′ is 0, 1, 2, or 3.

In some embodiments, R^{4′} is alkyl or haloalkyl, optionally substituted with one or more, the same or different R¹⁰. In some embodiments, R^{4′} is C₁-C₄ alkyl or C₁-C₄ haloalkyl, optionally substituted with one or more, the same or different R¹⁰. In some embodiments, R^{4′} is C₁-C₄ alkyl or C₁-C₄ haloalkyl, such as methyl.

In some embodiments, s′ is 1 and R⁴ is C₁-C₄ alkyl or C₁-C₄ haloalkyl, such as methyl.

Optionally, Formula IIIFC′ has the following configuration:

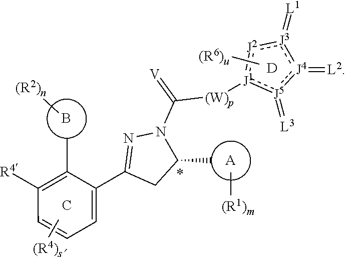

Optionally, the compounds have a structure of Formula IIIFD, an enantiomer or diastereomer thereof, or a pharmaceutically acceptable salt thereof,

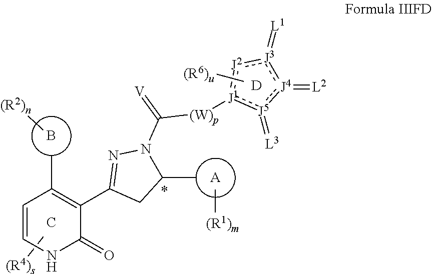

wherein s is 0, 1, 2, or 3; and
wherein ring A, ring B, ring D, T, T′, U, V W, R¹, R², R⁴, R⁵, R⁶, J¹, J², J³, J⁴, J⁵, L¹, L², L³, m, n, p, and u are the same as those described above in Formula IIIF.

Optionally, Formula IIIFD is in the following configuration:

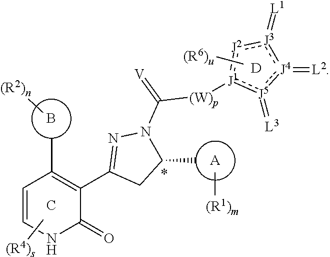

In some embodiments, Formula IIIFD is Formula IIIFD′, as shown below:

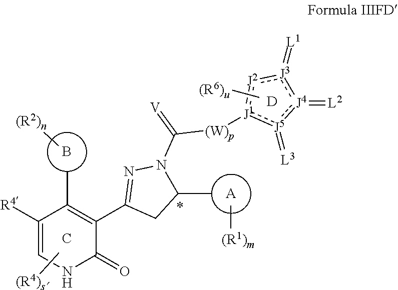

wherein R^{4′} has the same scope as R⁴; and
wherein s′ is 0, 1, or 2.

In some embodiments, R^{4′} is alkyl or haloalkyl, optionally substituted with one or more, the same or different R¹⁰. In some embodiments, R^{4′} is C₁-C₄ alkyl or C₁-C₄ haloalkyl, optionally substituted with one or more, the same or different R¹⁰. In some embodiments, R^{4′} is C₁-C₄ alkyl or C₁-C₄ haloalkyl, such as methyl.

In some embodiments, s′ is 1 and R⁴ is C₁-C₄ alkyl or C₁-C₄ haloalkyl, such as methyl.

Optionally, Formula IIIFD′ has the following configuration:

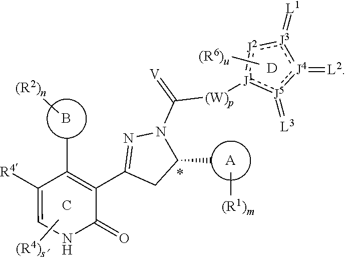

Optionally, the compounds have a structure of Formula IIIFE, an enantiomer or diastereomer thereof, or a pharmaceutically acceptable salt thereof,

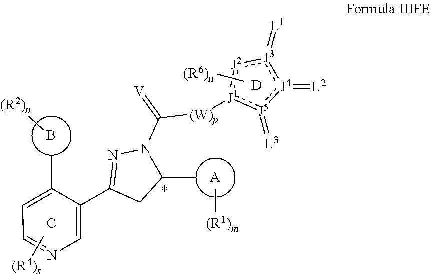

wherein s is 0, 1, 2, or 3; and
wherein ring A, ring B, ring D, T, T′, U, V W, R¹, R², R⁴, R⁵, R⁶, J¹, J², J³, J⁴, J⁵, L¹, L², L³, m, n, p, and u are the same as those described above in Formula IIIF.

Optionally, Formula IIIFE is in the following configuration:

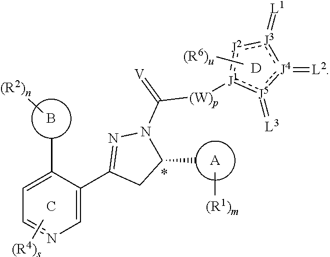

In some embodiments, Formula IIIFE is Formula IIIFE′, as shown below:

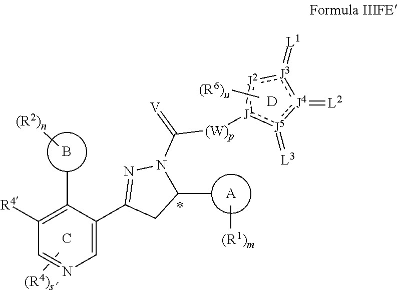

wherein R^{4′} has the same scope as R⁴; and
wherein s′ is 0, 1, or 2.

In some embodiments, R^{4′} is alkyl or haloalkyl, optionally substituted with one or more, the same or different R¹⁰. In some embodiments, R^{4′} is C₁-C₄ alkyl or C₁-C₄ haloalkyl, optionally substituted with one or more, the same or different R¹⁰. In some embodiments, R^{4′} is C₁-C₄ alkyl or C₁-C₄ haloalkyl, such as methyl.

In some embodiments, s′ is 1 and R⁴ is C₁-C₄ alkyl or C₁-C₄ haloalkyl, such as methyl.

Optionally, Formula IIIFE′ has the following configuration:

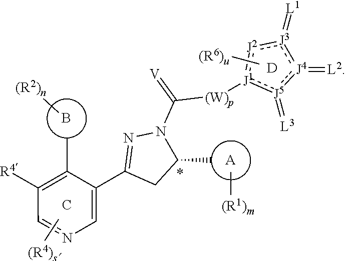

In some embodiments, the compounds are selected from the following and pharmaceutically acceptable salts thereof:

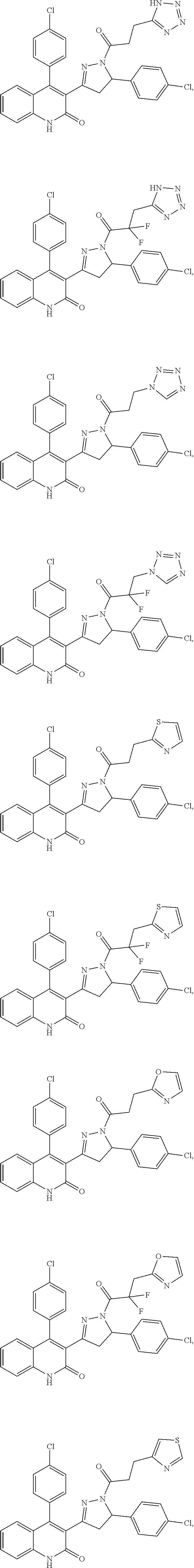

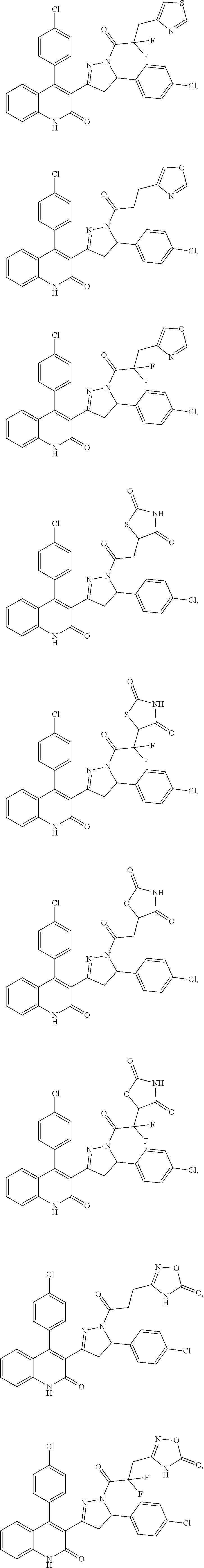

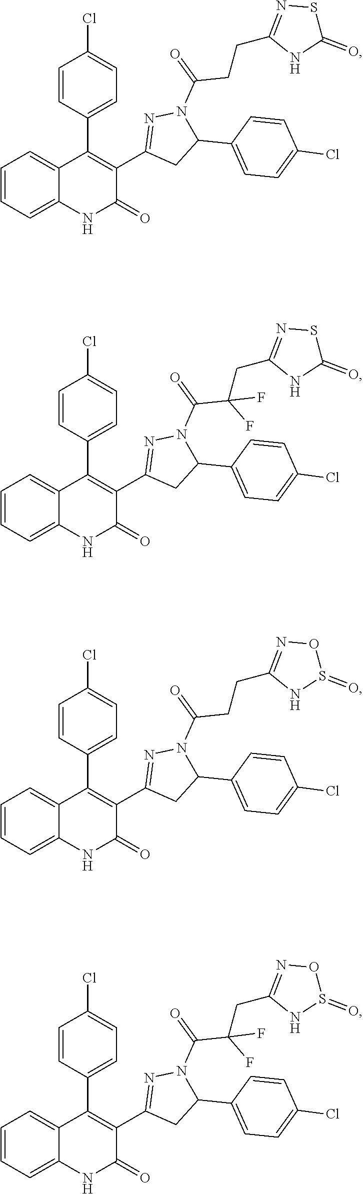 [SMILES: O=C(CCc1nsc(=O)[nH]1)N1N=C(c2c(-c3ccc(Cl)cc3)c3ccccc3[nH]c2=O)CC1c1ccc(Cl)cc1 ; O=C(N1N=C(c2c(-c3ccc(Cl)cc3)c3ccccc3[nH]c2=O)CC1c1ccc(Cl)cc1)C(F)(F)Cc1nsc(=O)[nH]1 ; O=C(CCC1=NOS(=O)N1)N1N=C(c2c(-c3ccc(Cl)cc3)c3ccccc3[nH]c2=O)CC1c1ccc(Cl)cc1 ; O=C(N1N=C(c2c(-c3ccc(Cl)cc3)c3ccccc3[nH]c2=O)CC1c1ccc(Cl)cc1)C(F)(F)CC1=NOS(=O)N1]

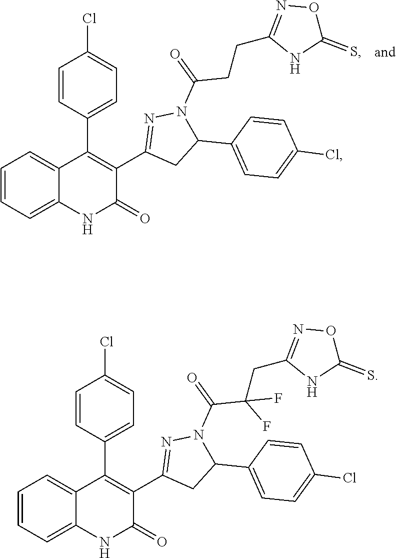 [SMILES: O=C(CCc1noc(=S)[nH]1)N1N=C(c2c(-c3ccc(Cl)cc3)c3ccccc3[nH]c2=O)CC1c1ccc(Cl)cc1 ; "and" ; O=C(N1N=C(c2c(-c3ccc(Cl)cc3)c3ccccc3[nH]c2=O)CC1c1ccc(Cl)cc1)C(F)(F)Cc1noc(=S)[nH]1]

The 5-membered nitrogen-containing heterocyclyl in the exemplary compounds above can be optionally substituted by one or more R⁶ where applicable.

In some embodiments, the compounds are selected from the following and pharmaceutically acceptable salts thereof:

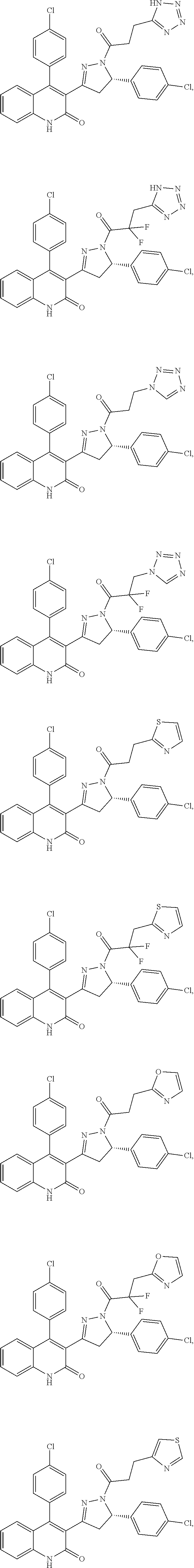

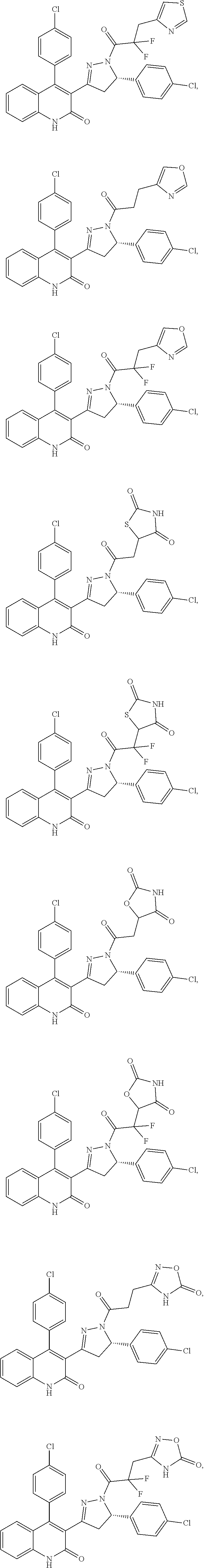

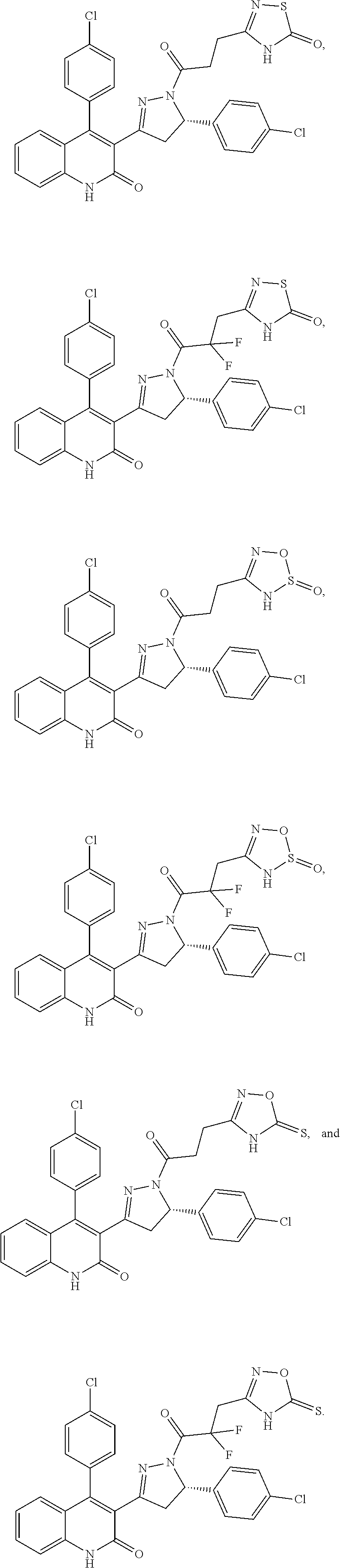

### III. Compositions

The disclosed compounds may be present in a mixture of stereoisomers. In some embodiments, the compounds in the mixture of stereoisomers may be in greater than 60%, 70%, 80%, 90%, 95%, or 98% diastereomeric or enantiomeric excess. In some embodiments, the compounds in the mixture of stereoisomers may be in greater than 95% diastereomeric or enantiomeric excess.

Disclosed are compositions containing the afore-mentioned mixture of stereoisomers. In some embodiments, the compositions contain a compound disclosed herein, wherein the compound is in greater than 60%, 70%, 80%, 90%, 95%, or 98% enantiomeric excess with respect to the stereocenter labeled by the “*” sign in Formulas I, IA, IB, IC, II, IIA, III, IIIA, IIIB, IIIC, IIIC′, HID, IIID′, IIIE, IIIE′, IIIF, IIIFA, IIIFB, IIIFB′, IIIFC, IIIFC′, IIIFD, IIIFD′, IIIFE, and IIIFE′.

For example, the compositions contain a compound of Formula I as disclosed herein, wherein the compound is in greater than 95% enantiomeric excess for the configuration depicted below:

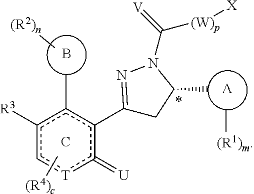

Another example, the compositions contain a compound of Formula II as disclosed herein, wherein the compound is in greater than 95% enantiomeric excess for the configuration depicted below:

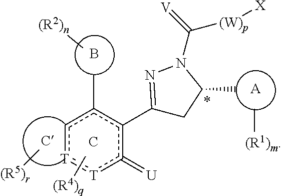

Another example, the compositions contain a compound of Formula III as disclosed herein, wherein the compound is in greater than 95% enantiomeric excess for the configuration depicted below:

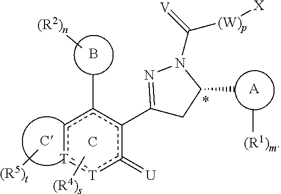

The disclosed compounds may be present in a mixture of the salt form and the non-salt form. In some embodiments, more than 50%, 60%, 70%, 80%, 90%, 95%, or 98% of the compound in the mixture may be in the salt form, calculated as the ratio of the weight of the salt form to the total weight of the salt form and the non-salt form. In some embodiments, more than 90% of the compound in the mixture may be in the salt form.

### IV. Formulations

Disclosed are pharmaceutical formulations containing a compound or composition described above. Generally, the pharmaceutical formulations also contain a pharmaceutically acceptable excipient. The pharmaceutical formulations may also include one or more further active agents or may be administered in combination with one or more such active agents.

The pharmaceutical formulations can be in the form of tablet, capsule, pill, caplets, cream, gel, granule, solution (such as aqueous solution, e.g., saline, buffered saline), emulsion, suspension, nanoparticle formulation, etc. In some embodiments, the pharmaceutical formulations are oral formulations. In some embodiments, the pharmaceutical formulations are intravenous formulations. In some embodiments, the pharmaceutical formulations are topical formulations.

The pharmaceutical formulations may be prepared in a manner known per se, which usually involves mixing a compound or composition according to the disclosure with the pharmaceutically acceptable excipient, and, if desired, in combination with other pharmaceutical active agent(s), when necessary under aseptic conditions.

As used herein, “emulsion” refers to a composition containing a mixture of non-miscible components homogenously blended together. In some forms, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil or an oleaginous substance is the dispersed liquid and water or an aqueous solution is the continuous phase, it is known as an oil-in-water emulsion, whereas when water or an aqueous solution is the dispersed phase and oil or an oleaginous substance is the continuous phase, it is known as a water-in-oil emulsion.

As used herein, “biocompatible” refers to materials that are neither themselves toxic to the host (e.g., a non-human animal or human), nor degrade (if the material degrades) at a rate that produces monomeric or oligomeric subunits or other byproducts at toxic concentrations in the host.

As used herein, “biodegradable” refers to materials degrade or break down into its component subunits, or digestion, e.g., by a biochemical process, of the materials into smaller (e.g., non-polymeric) subunits.

As used herein, “enteric polymers” refer to polymers that become soluble in the higher pH environment of the lower gastrointestinal tract or slowly erode as the dosage form passes through the gastrointestinal tract.

As used herein, “enzymatically degradable polymers” refer to polymers that are degraded by bacterial enzymes present in the lower gastrointestinal tract, particularly in the colon.

As used herein, “pharmaceutically acceptable” refers to compounds, materials, compositions, and/or formulations which are, within the scope of sound medical judgment, suitable for use in contact with the tissues of human beings and animals without excessive toxicity, irritation, allergic response, or other problems or complications that commensurate with a reasonable benefit/risk ratio, in accordance with the guidelines of agencies such as the Food and Drug Administration (FDA) and its foreign equivalents.

As used herein, “nanoparticle” generally refers to particles having a diameter from about 1 nm to 1000 nm, preferably from about 10 nm to 1000 nm, more preferably from about 100 nm to 1000 nm, most preferably from about 250 nm to 1000 nm. In some embodiments, “nanoparticles” can also refer to “microparticles,” which are particles having a diameter from about 1 micron to about 100 microns, preferably from about 1 to about 50 microns, more preferably from about 1 to about 30 microns, most preferably from about 1 micron to about 10 microns. In some embodiments, the nanoparticles can be a mixture of nanoparticles, as defined above, and microparticles, as defined above.

As used herein, the term “surfactant” refers to any agent which preferentially absorbs to an interface between two immiscible phases, such as the interface between water and an organic polymer solution, a water/air interface, or organic solvent/air interface. Surfactants generally possess a hydrophilic moiety and a lipophilic moiety.

As used herein, “gel” is a semisolid system containing a dispersion of the active agent, i.e., the compound, in a liquid vehicle that is rendered semisolid by the action of a thickening agent or polymeric material dissolved or suspended in the liquid vehicle. The liquid vehicle may include a lipophilic component, an aqueous component or both.

As used herein, “hydrogel” refers to a swollen, water-containing network of finely-dispersed polymer chains that are water-insoluble, where the polymeric molecules are in the external or dispersion phase and water (or an aqueous solution) forms the internal or dispersed phase. The chains can be chemically cross-linked (chemical gels) or physically cross-linked (physical gels). Chemical gels possess polymer chains that are connected through covalent bonds, whereas physical gels have polymer chains linked by non-covalent bonds or cohesion forces, such as van der Waals interactions, ionic interactions, hydrogen bonding interactions, and/or hydrophobic interactions.

As used herein, drug-containing “beads” refer to beads made with drug and one or more excipients. Drug-containing beads can be produced by applying the drug to an inert support, e.g., inert sugar beads coated with the drug, or by creating a “core” comprising both the drug and the one or more excipients. As is also known, drug-containing “granules” and “particles” comprise the drug and optionally one or more excipients. Typically, drug-containing granules and particles do not contain an inert support. Drug-containing granules generally comprise drug-containing particles and require further processing. Generally, drug-containing particles are smaller than drug-containing granules and are not further processed. Although drug-containing beads, granules, and particles, as described above, may be formulated to provide immediate release, drug-containing beads and drug-containing granules are generally employed to provide delayed release.

### A. Physical Forms and Unit Dosages

Depending upon the manner of introduction, the compounds or compositions described herein may be formulated in a variety of ways. The pharmaceutical formulations can be prepared in various forms, such as granules, tablets, capsules, pills, caplets, suppositories, powders, controlled release formulations, nanoparticle formulations, solutions (such as aqueous solutions, e.g., saline, buffered saline), syrups, suspensions, emulsions, creams, gels, ointments, salves, lotions, aerosols, and the like.

In some embodiments, the pharmaceutical formulations are in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspensions (including nano- or micro-suspensions), shakes, emulsions, etc., can also be utilized. Intravenous formulations are usually in liquid dosage forms, including solutions, emulsions, and suspensions. Suitable topical formulations include, but are not limited to, lotions, ointments, creams, and gels. In some embodiments, the topical formulations are in the form of gels or creams.

The concentration of the compound to the pharmaceutically acceptable excipient may vary from about 0.5 to about 100 wt %. For oral use, the pharmaceutical formulations generally contain from about 5 to about 100 wt % of the compound. For other uses, the pharmaceutical formulations generally have from about 0.5 to about 50 wt % of the compound.

In some embodiments, the pharmaceutical formulations are in a unit dosage form, and may be suitably packaged, for example in a box, blister, vial, bottle, sachet, ampoule or in any other suitable single-dose or multi-dose holder or container (which may be properly labeled), optionally with one or more leaflets containing product information and/or instructions for use. Generally, such unit dosages contain between 1 and 1000 mg, and usually between 5 and 500 mg, of a compound from the disclosure, e.g., about 10, 25, 50, 100, 200, 300 or 400 mg per unit dosage.

### B. Pharmaceutically Acceptable Excipients

As used herein, “excipient” refers to all components present in the pharmaceutical formulations other than the active ingredient(s). Pharmaceutically acceptable excipients are composed of materials that are considered safe and may be administered to an individual without causing undesirable biological side effects or unwanted interactions. For example, the pharmaceutically acceptable excipients can be compounds or materials recognized by the FDA as “generally recognized as safe” or “GRAS”.

Generally, excipients include, but are not limited to, diluents (fillers), binders, lubricants, disintegrants, pH modifying or buffering agents, preservatives, antioxidants, solubility enhancers, wetting or emulsifying agents, plasticizers, colorants (such as pigments and dyes), stabilizers, glidants, solvent or dispersion medium, surfactants, pore formers, and coating or matrix materials.

In some embodiments, drug-containing tablets, beads, granules, or particles contain one or more of the following excipients: diluents, binders, lubricants, disintegrants, pigments, stabilizers, and surfactants. If desired, the tablets, beads, granules, or particles may also contain minor amount of nontoxic auxiliary substances such as wetting or emulsifying agents, dyes, pH buffering agents, or preservatives.

Examples of coating or matrix materials include, but are not limited to, cellulose polymers (such as methylcellulose, ethyl cellulose, cellulose acetate, cellulose acetate phthalate, hydroxypropyl cellulose, hydroxyethyl cellulose, hydroxymethyl cellulose, hydroxypropyl methylcellulose, hydroxypropyl methylcellulose phthalate, hydroxypropyl methylcellulose acetate succinate, cellulose acetate trimellitate, and carboxymethylcellulose sodium), vinyl polymers and copolymers (such as polyvinyl pyrrolidone, polyvinyl acetate, polyvinyl acetate phthalate, vinyl acetate-crotonic acid copolymer, and ethylene-vinyl acetate copolymer), acrylic acid polymers and copolymers (such as those formed from acrylic acid, methacrylic acid, methyl acrylate, ethyl acrylate, methyl methacrylate and/or ethyl methacrylate, and other methacrylic resins that are commercially available under the tradename EUDRAGIT®), enzymatically degradable polymers (such as azo polymers, pectin, chitosan, amylose, and guar gum), zein, shellac, and polysaccharides. In some embodiments, the coating or matrix materials may contain conventional excipients such as plasticizers, colorants, glidants, stabilizers, pore formers, and surfactants.

In some embodiments, the coating or matrix materials are pH-sensitive or pH-responsive polymers, such as the enteric polymers commercially available under the tradename EUDRAGIT®. For example, EUDRAGIT® L30D-55 and L100-55 are soluble at pH 5.5 and above; EUDRAGIT® L100 is soluble at pH 6.0 and above; EUDRAGIT® S is soluble at pH 7.0 and above, as a result of a higher degree of esterification.

In some embodiments, the coating or matrix materials are water-insoluble polymers having different degrees of permeability and expandability, such as EUDRAGIT® NE, RL, and RS.

Depending on the coating or matrix materials, the decomposition/degradation or structural change of the pharmaceutical formulations may occur at different locations of the gastrointestinal tract. In some embodiments, the coating or matrix materials are selected such that the pharmaceutical formulations can survive exposure to gastric acid and release the compound in the intestines after oral administration.

Diluents, also referred to as “fillers,” can increase the bulk of a solid dosage form so that a practical size is provided for compression of tablets or formation of drug-containing beads, granules, or particles. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, powdered sugar, and combinations thereof.

Binders are used to impart cohesive qualities to a solid dosage formulation, and thus ensure that a drug-containing tablet, bead, granule, or particle remains intact after the formation of the dosage forms. Suitable binder materials include, but are not limited to, starch, pregelatinized starch, gelatin, sugars (such as sucrose, glucose, dextrose, lactose and sorbitol), polyethylene glycol, waxes, natural and synthetic gums (such as acacia, tragacanth, and sodium alginate), cellulose (such as hydroxypropylmethylcellulose, hydroxypropylcellulose, and ethylcellulose), veegum, synthetic polymers (such as acrylic acid and methacrylic acid copolymers, methacrylic acid copolymers, methyl methacrylate copolymers, aminoalkyl methacrylate copolymers, polyacrylic acid, polymethacrylic acid, and polyvinylpyrrolidone), and combinations thereof.

Lubricants are used to facilitate tablet manufacture. Suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, gums, and cross-linked polymers, such as cross-linked polyvinylpyrrolidone (e.g., POLYPLASDONE® XL from GAF Chemical Corp.).

Plasticizers are normally present to produce or promote plasticity and flexibility and to reduce brittleness. Examples of plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides.

Stabilizers are used to inhibit or retard decomposition reactions of the active agents in the formulations or stabilize particles in a dispersion, For example, when the decomposition reactions involve an oxidation reaction of an active agent in the formulations, the stabilizer can be an antioxidant or a reducing agent. Stabilizers also include nonionic emulsifiers such as sorbitan esters, polysorbates, and polyvinylpyrrolidone.

Glidants are used to reduce sticking effects during film formation and drying. Exemplary glidant include, but are not limited to talc, magnesium stearate, and glycerol monostearates.

Pigments such as titanium dioxide may also be used.

Preservatives can inhibit the deterioration and/or decomposition of a pharmaceutical formulation. Deterioration or decomposition can be brought about by any of microbial growth, fungal growth, and undesirable chemical or physical changes. Suitable preservatives include benzoate salts (e.g., sodium benzoate), ascorbic acid, methyl hydroxybenzoate, ethyl p-hydroxybenzoate, n-propyl p-hydroxybenzoate, n-butyl p-hydroxybenzoate, potassium sorbate, sorbic acid, proprionate salts (e.g., sodium propionate), chlorobutanol, benzyl alcohol, and combinations thereof.

Surfactants may be anionic, cationic, amphoteric or nonionic surface-active agents. Exemplary anionic surfactants include, but are not limited to, those containing carboxylate, sulfonate, and sulfate ions. Examples of anionic surfactants include sodium, potassium, ammonium of long chain (e.g., 13-21) alkyl sulfonates; alkyl aryl sulfonates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, and polyoxyethylene coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, polyxamers (such as poloxamer 401), stearoyl monoisopropanolamide, and polyoxyethylene hydrogenated tallow amide. Examples of amphoteric surfactants include, but are not limited to, sodium N-dodecyl-β-alanine, sodium N-lauryl-β-iminodipropionate, myristoamphoacetate, lauryl betaine, and lauryl sulfobetaine.

Pharmaceutical formulations in liquid forms typically contain a solvent or dispersion medium such as water, aqueous solution (such as saline and buffered saline), ethanol, polyol (such as glycerol, propylene glycol, and liquid polyethylene glycol), oil (such as vegetable oil, e.g., peanut oil, corn oil, sesame oil, etc.), and combinations thereof. Preferably, the pharmaceutical formulations in liquid forms are aqueous formulations. Suitable solvents or dispersion media for intravenous formulations include, but are not limited to, water, saline, buffered saline (such as phosphate buffered saline), and Ringer's solution.

### C. Pharmaceutical Acceptable Carriers

In some embodiments, the pharmaceutical formulations are prepared using a pharmaceutically acceptable carrier, which encapsulates, embeds, entraps, dissolves, disperses, absorbs, or bind to a compound or composition disclosed herein. The pharmaceutical acceptable carrier is composed of materials that are considered safe and can be administered to a subject without causing undesirable biological side effects or unwanted interactions. Preferably, the pharmaceutically acceptable carrier does not interfere with the effectiveness of the compound or composition in performing its function. The pharmaceutically acceptable carrier can be formed of biodegradable materials, non-biodegradable materials, or combinations thereof. The pharmaceutical acceptable excipient described above may be partially or entirely present in the pharmaceutical acceptable carrier.

In some embodiments, the pharmaceutical acceptable carrier is a controlled-release carrier, such as delayed-release carriers, sustained-release (extended-release) carriers, and pulsatile-release carriers.

In some embodiments, the pharmaceutical acceptable carrier is pH-sensitive or pH-responsive. In some forms, the pharmaceutical acceptable carrier can decompose or degrade in a certain pH range. In some forms, the pharmaceutical acceptable carrier can experience a structural change when experiencing a change in the pH.

Exemplary pharmaceutical acceptable carriers include, but are not limited to, nanoparticles, liposomes, hydrogels, polymer matrices, and solvent systems.

In some embodiments, the pharmaceutical acceptable carrier is nanoparticles. In some forms, the compound is embedded in the matrix formed by materials of the nanoparticles.

The nanoparticles can be biodegradable, and preferably are capable of biodegrading at a controlled rate for delivery of the compound. The nanoparticles can be made of a variety of materials. Both inorganic and organic materials can be used. Both polymeric and non-polymeric materials can be used.

Preferably, the nanoparticles are polymeric nanoparticles formed of one or more biocompatible polymers, copolymers, or blends thereof. In some forms, the biocompatible polymers are biodegradable. In some forms, the biocompatible polymers are non-biodegradable.

In some forms, the nanoparticles are formed of a mixture of biodegradable and non-biodegradable polymers. The polymers may be tailored to optimize different characteristics of the nanoparticles including: (i) interactions between the compound and the polymer to provide stabilization of the compound and retention of activity upon delivery; (ii) rate of polymer degradation and, thereby, rate of release; (iii) surface characteristics and targeting capabilities via chemical modification; and (iv) particle porosity.

Exemplary polymers include, but are not limited to, polymers prepared from lactones such as poly(caprolactone) (PCL), polyhydroxy acids and copolymers thereof such as poly(lactic acid) (PLA), poly(glycolic acid) (PGA), poly(lactic acid-co-glycolic acid) (PLGA), and blends thereof, polyalkyl cyanoacralate, polyurethanes, polyamino acids such as poly-L-lysine (PLL), poly(valeric acid), and poly-L-glutamic acid, hydroxypropyl methacrylate (HPMA), polyanhydrides, polyorthoesters, poly(ester amides), polyamides, poly(ester ethers), polycarbonates, ethylene vinyl acetate polymer (EVA), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), celluloses including derivatized celluloses such as alkyl celluloses, hydroxyalkyl celluloses, cellulose ethers, cellulose esters, nitro celluloses, hydroxypropylcellulose, and carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”), polydioxanone and its copolymers, polyhydroxyalkanoates, polypropylene fumarate, polyoxymethylene, poloxamers, poly(butyric acid), trimethylene carbonate, polyphosphazenes, polysaccharides, peptides or proteins, and copolymers or blends thereof.

Preferably, the polymer is an FDA approved biodegradable polymer such as polyhydroxy acids (e.g., PLA, PLGA, PGA), polyanhydride, polyhydroxyalkanoate such as poly(3-butyrate) or poly(4-butyrate), and copolymer or blends thereof.

Materials other than polymers may be used to form the nanoparticles. Suitable materials include excipients such as surfactants.

The use of surfactants in the nanoparticles may improve surface properties by, for example, reducing particle-particle interactions, and render the surface of the particles less adhesive. Both naturally occurring surfactants and synthetic surfactants can be incorporated into the nanoparticles. Exemplary surfactants include, but are not limited to, phosphoglycerides such as phosphatidylcholines (e.g., L-α-phosphatidylcholine dipalmitoyl), diphosphatidyl glycerol, hexadecanol, fatty alcohols, polyoxyethylene-9-lauryl ether, fatty acids such as palmitic acid or oleic acid, sorbitan trioleate, glycocholate, surfactin, poloxomers, sorbitan fatty acid esters such as sorbitan trioleate, tyloxapol, and phospholipids.

The nanoparticles can contain a plurality of layers. The layers can have similar or different release kinetic profiles for the compound. For example, the nanoparticles can have a controlled-release core surrounded by one or more additional layers. The one or more additional layers can include an instant-release layer, preferably on the surface of the nanoparticles. The instant-release layer can provide a bolus of the compound shortly after administration.

The composition and structure of the nanoparticles can be selected such that the nanoparticles are pH-sensitive or pH-responsive. In some forms, the particles are formed of pH-sensitive or pH-responsive polymers such as the enteric polymers commercially available from under the tradename EUDRAGIT®, as described above. Depending on the particle materials, the decomposition/degradation or structural change of the nanoparticles may occur at different locations of the gastrointestinal tract. In some embodiments, the particle materials are selected such that the pharmaceutical formulations can survive exposure to gastric acid and release the compound in the intestines after oral administration.

### D. Controlled Release

In some embodiments, the pharmaceutical formulations can be controlled release formulations. Examples of controlled release formulations include extended-release formulations, delayed release formulations, pulsatile release formulations, and combinations thereof. In some embodiments, each dosage unit in capsule may contain a plurality of drug-containing beads, granules, or particles, having different release profiles.

### 1. Extended Release

In some embodiments, the extended-release formulations are prepared as diffusion or osmotic systems, for example, as described in “Remington—The science and practice of pharmacy” (20th Ed., Lippincott Williams & Wilkins, 2000).

A diffusion system is typically in the form of a matrix, generally prepared by compressing the drug with a slowly dissolving carrier, optionally into a tablet form. The three major types of materials used in the preparation of the matrix are insoluble plastics, hydrophilic polymers, and fatty compounds. Plastic matrices include, but are not limited to, methyl acrylate-methyl methacrylate copolymer, polyvinyl chloride, and polyethylene. Hydrophilic polymers include, but are not limited to, cellulosic polymers such as methyl ethyl cellulose, hydroxyalkylcelluloses (such as hydroxypropylcellulose, hydroxypropylmethylcellulose), sodium carboxymethylcellulose, CARBOPOL® 934, polyethylene oxides, and mixtures thereof. Fatty compounds include, but are not limited to, various waxes such as carnauba wax and glyceryl tristearate, wax-type substances including hydrogenated castor oil and hydrogenated vegetable oil, and mixtures thereof.

In some embodiments, the plastic material is a pharmaceutically acceptable acrylic polymer, including but not limited to, acrylic acid and methacrylic acid copolymers, methyl methacrylate copolymers, ethoxyethyl methacrylate copolymers, cyanoethyl methacrylate copolymers, aminoalkyl methacrylate copolymers, poly(acrylic acid), poly(methacrylic acid), methacrylic acid alkylamine copolymers, poly(methyl methacrylate), poly(methacrylic acid)(anhydride), polymethacrylate, polyacrylamide, poly(methacrylic acid anhydride), and glycidyl methacrylate copolymers.

In some embodiments, the acrylic polymer can be an ammonio methacrylate copolymer. Ammonio methacrylate copolymers are well known in the art and are described as fully polymerized copolymers of acrylic and methacrylic acid esters with a low content of quaternary ammonium groups.

In some embodiments, the acrylic polymer is an acrylic resin lacquer such as those commercially available from under the tradename EUDRAGIT®. In some embodiments, the acrylic polymer contains a mixture of two acrylic resin lacquers, EUDRAGIT® RL30D and EUDRAGIT® RS30D. EUDRAGIT® RL30D and EUDRAGIT® RS30D are copolymers of acrylic and methacrylic esters with a low content of quaternary ammonium groups, the molar ratio of ammonium groups to the remaining neutral methacrylic esters being 1:20 in EUDRAGIT® RL30D and 1:40 in EUDRAGIT® RS30D. In some embodiments, the mean molecular weight for both copolymers is about 150,000, The code designations RL (high permeability) and RS (low permeability) refer to the permeability properties of these polymers. EUDRAGIT® RL/RS mixtures are insoluble in water and in digestive fluids. However, multiparticulate systems formed to include the same are swellable and permeable in aqueous solutions and digestive fluids. In some embodiments, the acrylic polymer can also be or include other EUDRAGIT® acrylic resin lacquers, such as EUDRAGIT® S-100, EUDRAGIT® L-100, or a mixture thereof.

The polymers described above such as EUDRAGIT® RL/RS may be mixed in any desired ratio in order to ultimately obtain a sustained-release formulation having a desirable dissolution profile. Desirable sustained release multiparticulate systems may be obtained, for instance, from 100% EUDRAGIT® RL, to 50% EUDRAGIT® RL+50% EUDRAGIT® RS, and to 10% EUDRAGIT® RL+90% EUDRAGIT® RS.

Matrices with different drug release mechanisms described above can be combined in a final dosage form containing single or multiple units. Examples of multiple units include, but are not limited to, multilayer tablets and capsules containing tablets, beads, or granules. An immediate release portion can be added to the extended-release system by means of either applying an immediate release layer on top of the extended-release core using a coating or compression process or in a multiple unit system such as a capsule containing extended and immediate release beads.

Extended-release tablets containing hydrophilic polymers are prepared by techniques commonly known in the art such as direct compression, wet granulation, or dry granulation.

Extended-release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Alternatively, extended-release formulations can be prepared using osmotic systems or by applying a semi-permeable coating to a solid dosage form. In the latter case, the desired drug release profile can be achieved by combining low permeable and high permeable coating materials in suitable proportion.

### 2. Delayed Release

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a coating material. The drug-containing composition may be a tablet for incorporation into a capsule, a tablet for use as an inner core in a “coated core” dosage form, or a plurality of drug-containing beads, particles, or granules, for incorporation into either a tablet or capsule. Suitable coating materials include bioerodible, gradually hydrolyzable, gradually water-soluble, and/or enzymatically degradable polymers, such as those described above. In some embodiments, the coating material is or contains enteric polymers. Combinations of different coating materials may also be used. Multilayer coatings using different polymers may also be applied.

The preferred coating weights for particular coating materials may be readily determined by those skilled in the art by evaluating individual release profiles for tablets, beads and granules prepared with different quantities of the coating materials.

The coating materials may contain conventional additives, such as plasticizers (generally represent about 10 wt % to 50 wt % relative to the dry weight of the coating material), colorants, stabilizers, glidants, etc., such as those described above.

### 3. Pulsatile Release

Pulsatile-release formulations release a plurality of drug doses at spaced-apart time intervals. Generally, upon administration, such as ingestion, of the pulsatile-release formulations, release of the initial dose is substantially immediate, e.g., the first drug release “pulse” occurs within about one hour of administration. This initial pulse is followed by a first time-interval (lag time) during which very little or no drug is released from the formulations, after which a second dose is then released. Similarly, a second lag time (nearly drug release-free interval) between the second and third drug release pulses may be designed. The duration of the lag times will vary depending on the formulation design, especially on the length of the desired drug administration interval, e.g., a twice daily dosing profile, a three times daily dosing profile, etc.

For pulsatile-release formulations providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In some forms, the pulsatile-release formulations contain a plurality of drug carriers with different drug-release kinetics.

In some forms, the pulsatile-release formulations contain a drug carrier with a plurality of drug-loaded layers. The drug-loaded layers may have different drug release kinetics. The layers may be separated by a delayed-release coating. For example, the carrier may have a drug-loaded layer on the surface for the first pulse and a drug-loaded core for the second pulse; the drug-loaded core may be surrounded by a delayed-release coating, which creates a lag time between the two pulses.

In some embodiments, the pulsatile release profile is achieved with formulations that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the drug in a controlled release polymer matrix. Each dosage unit may comprise a compressed or molded tablet, wherein each tablet within the capsule provides a different drug release profile.

### E. Exemplary Formulations for Different Routes of Administration

A subject suffering from schizophrenia, Parkinson's disease, depression, obsessive-compulsive disorders, neuropathic pain, stroke, traumatic brain injury, epilepsy, or other neurologic events or neurodegeneration involving NMDA GluN2 receptor activation, especially NMDA GluN2C/D receptor activation, can be treated by either targeted or systemic administration, via oral, inhalation, topical, trans- or sub-mucosal, subcutaneous, parenteral, intramuscular, intravenous or transdermal administration of a formulation comprising an effective amount of one or more compounds described herein, optionally in a pharmaceutically acceptable carrier. The compounds are typically administered by oral administration. Alternatively, the compounds can be administered by inhalation or intranasal routes. Alternatively, the compounds are administered transdermally (for example via a slow-release patch) or topically. In yet another embodiment, the compounds are administered subcutaneously, intravenously, intraperitoneally, intramuscularly, parenterally, or submucosally.

In some embodiments, the compounds are administered orally. Oral formulations will generally include an inert diluent or an edible carrier. They may be enclosed in gelatin capsules or compressed into tablets. For the purpose of oral administration, the active compound can be incorporated with one or more excipients as described above and used in the form of tablets, pills, troches, or capsules.

When the compounds are administered orally in the form of tablets, pills, capsules, troches and the like, the corresponding oral formulations can contain any of the following ingredients or compositions of a similar nature: a binder as described above, a disintegrant as described above, a lubricant as described above, a glidant as described above, a sweetening agent (such as sucrose or saccharin), and/or a flavoring agent (such as peppermint, methyl salicylate, or orange flavoring). When the oral formulation is in the form of capsule, it can contain, in addition to materials listed above, a liquid carrier (such as a fatty oil). In addition, the oral formulations can contain various other materials which modify the physical form of the dosage unit, for example, coatings of polysaccharides, shellac, or enteric agents as described in previous sections.

The compound can also be administered orally as a component of an elixir, suspension, syrup, wafer, chewing gum or the like. A syrup may contain, in addition to the active compounds, a sweetening agent (such as sucrose, saccharine, etc.) and preservatives, dyes and colorings and flavors.

The compounds can also be administered in specific, measured amounts in the form of an aqueous suspension by use of a pump spray bottle. This administration route is suitable for intranasal administration. The aqueous suspension compositions may be prepared by admixing the compounds with water and other pharmaceutically acceptable excipients. The aqueous suspension compositions may contain water, auxiliaries and/or one or more of the excipients, such as: suspending agents, e.g., microcrystalline cellulose, sodium carboxymethylcellulose, hydroxypropyl-methyl cellulose; humectants, e.g. glycerol and propylene glycol; acids, bases or buffer substances for adjusting the pH, e.g., citric acid, sodium citrate, phosphoric acid, sodium phosphate as well as mixtures of citrate and phosphate buffers; surfactants, e.g. Polysorbate 80; and antimicrobial preservatives, e.g., benzalkonium chloride, phenylethyl alcohol and potassium sorbate.

In some embodiments, the compounds are in the form of an inhaled dosage for administration via inhalation. For example, the compounds may be in the form of an aerosol suspension, a dry powder or liquid particle form. The compounds may be prepared for delivery as a nasal spray or in an inhaler, such as a metered dose inhaler. Pressurized metered-dose inhalers (“MDI”) generally deliver aerosolized particles suspended in chlorofluorocarbon propellants such as CFC-11, CFC-12, or the non-chlorofluorocarbons or alternate propellants such as the fluorocarbons, HFC-134A or HFC-227 with or without surfactants and suitable bridging agents. Dry-powder inhalers can also be used, either breath activated or delivered by air or as pressure such as the dry-powder inhaler.

A parenteral preparation can be enclosed in ampoules, disposable syringes or multiple dose vials made of glass or plastic.

If administered intravenously, carriers of the compounds can be physiological saline, bacteriostatic water, Cremophor ELTM (BASF, Parsippany, NJ), or phosphate buffered saline (PBS).

Suitable vehicles or carriers for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients, and stabilizers can be used to prepare topical compositions. Examples of thickening agents include petrolatum, beeswax, xanthan gum, or polyethylene, humectants such as sorbitol, emollients such as mineral oil, lanolin and its derivatives, or squalene.

In some embodiments, the compounds are prepared with carriers that will protect them against rapid elimination from the body, such as a controlled release formulation as described in previous sections.

### V. Methods of Using

Disclosed are methods of treating a neurological condition or disorder in a subject in need thereof. The methods include administering an effective amount of a compound disclosed herein to the subject.

In some embodiments, the compound is administered in the form of a pharmaceutical formulation, such as those described above. The compound or pharmaceutical formulation can be administered in a variety of manners, depending on whether local or systemic administration is desired. In some embodiments, the compound is directly administered to a specific bodily location of the subject, e.g., topically administration. In some embodiments, the compound is administered in a systemic manner, such as enteral administration (e.g., oral administration) or parenteral administration (e.g., injection, infusion, and implantation). Exemplary administration routes include oral administration, intravenous administration such as intravenous injection or infusion, and topical administration. In some embodiments, the compound is administered orally. In some embodiments, the compound is administered intravenously. In some embodiments, the compound is administered intranasally.

As used herein, “effective amount” of a material refers to a nontoxic but sufficient amount of the material to provide the desired result. The exact amount required may vary from subject to subject, depending on the species, age, and general condition of the subject, the severity of the disease, disorder, or condition that is being treated, the particular drug or therapy used, its mode of administration, and the like.

### A. Indications

In general, the neurological condition or disorder is mediated by abnormality in GluN2-containing NMDARs, especially those containing GluN2C or GluN2D. The abnormality in GluN2-containing NMDARs can be caused by or associated with elevated expression levels and/or up-regulated activity of these receptors.

Exemplary neurological conditions or disorders in this context include, but are not limited to, (1) major mental disorders (such as depression, bipolar disorder, attention-deficit disorder, schizophrenia, anxiety, various psychoses, compulsivity and related disorders, and epilepsies), (2) conditions that involve basal ganglia or altered dopamine (such as dystonia and related motor disorders, Huntingtin's disease, Parkinson's disease, and L-DOPA-induced dyskinesias or dyskinesias that result from medication), (3) substance abuse/addiction or predisposition to substance abuse/addiction, (4) pain disorders, (5) developmental delay or situations with impaired learning, memory, and/or cognition (such as neurodegenerative diseases, e.g., Alzheimer's disease, Parkinson's disease, frontal lobe dementia, and motor retraining after acute injury), (6) acute neuronal or glial injuries (such as those that occur during hypoxia, ischemia, stroke, periventricular leukomalacia, traumatic brain injury, spinal cord injury, acute injury involving the white matter, neonatal seizures, and status epilepticus), and (7) circuit disorders (such as Huntington's disease).

In some embodiments, the compounds are used in a method of treatment or prophylaxis of schizophrenia, Parkinson's disease, bipolar disorder, depression, anxiety, neuropsychiatric or mood disorders, obsessive-compulsive disorder, motor dysfunction, neuropathic pain, ischemic and hemorrhagic stroke, subarachnoid hemorrhage, cerebral vasospasm, ischemia, hypoxia, Alzheimer's disease, pre-senile dementia, amyotrophic lateral sclerosis (ALS), Huntington's chorea, traumatic brain injury, epilepsy, or other neurologic events, neurocognitive disorders, tardive dyskinesia, motor disorders, mood disorders or neurodegeneration involving NMDAR activation.

For example, the compounds are used to treat Parkinson's disease or reduce one or more symptoms of Parkinson's disease. Exemplary symptoms include dystonia and related movement disorders.

In some embodiments, the compounds are used to provide cognitive enhancement, in normal or cognitively deficient individuals.

In some embodiments, the compounds are used to treat or prevent stroke or stroke-associated damages and can be administered under emergency care for a stroke, for maintenance treatment of stroke, and/or for rehabilitation of stroke. In some embodiments, the stroke includes vessel occlusion due to atherosclerosis, thrombotic emboli, or a vascular disease that leads to vessel occlusion without bleeding.

In some embodiments, the compounds are used to treat patients with ischemic injury or hypoxia, or to prevent or treat the neuronal toxicity associated with ischemic injury or hypoxia. In some embodiments, the ischemic injury is produced by vascular pathology, thrombotic emboli, atherosclerosis, or other vascular abnormalities that reduces cerebral blood flow in a region or completely occludes a cerebral blood vessel either transiently or permanently. In some embodiments, the ischemic injury is selected from, but not limited to, one of the following: traumatic brain injury, cognitive deficit after bypass surgery, cognitive deficit after carotid angioplasty, and neonatal ischemia following hypothermic circulatory arrest. Optionally, the bypass surgery is a cardiac bypass—a surgery involving cardiac bypass pump. In another embodiments, the ischemic injury is vasospasm after subarachnoid hemorrhage.

A subarachnoid hemorrhage refers to an abnormal condition in which blood collects beneath the arachnoid mater, a membrane that covers the brain. This area, called the subarachnoid space, normally contains cerebrospinal fluid. The accumulation of blood in the subarachnoid space and the vasospasm of the vessels which results from it can lead to stroke, seizures, and other complications. The methods and compounds described herein can be used to treat patients experiencing a subarachnoid hemorrhage. In one embodiment, the methods and compounds described herein can be used to limit the toxic effects of hemorrhage, including, for example, stroke and/or ischemia that can result from the subarachnoid hemorrhage. In a particular embodiment, the methods and compounds described herein can be used to treat patients with traumatic subarachnoid hemorrhage. For example, the traumatic subarachnoid hemorrhage can be due to a head injury. In another embodiment, the methods and compounds described herein can be used to treat patients with spontaneous subarachnoid hemorrhage.

In some embodiments, methods are provided to treat patients with neuropathic pain. In certain embodiments, the neuropathic pain or related disorder can be selected from, but not limited to, peripheral diabetic neuropathy, postherpetic neuralgia, complex regional pain syndromes, peripheral neuropathies, chemotherapy-induced neuropathic pain, cancer neuropathic pain, neuropathic low back pain, HIV neuropathic pain, trigeminal neuralgia, and central post-stroke pain.

Neuropathic pain can be associated with signals generated ectopically and often in the absence of ongoing noxious events by pathologic processes in the peripheral or central nervous system. Alternatively, neuropathic pain can result from peripheral or central nervous system pathologic events, including, but not limited to trauma; ischemia; ongoing metabolic or toxic diseases, infections or endocrinologic disorders, including, but not limited to, diabetes mellitus, diabetic neuropathy, amyloidosis, amyloid polyneuropathy (primary and familial), neuropathies with monoclonal proteins, vasculitic neuropathy, HIV infection, herpes zoster shingles, and postherpetic neuralgia; neuropathy associated with Guillain-Barre syndrome; neuropathy associated with Fabry's disease; entrapment due to anatomic abnormalities; trigeminal and other CNS neuralgias; malignancies; inflammatory conditions or autoimmune disorders, including, but not limited to, demyelinating inflammatory disorders, rheumatoid arthritis, systemic lupus erythematosus, and Sjogren's syndrome; and cryptogenic causes, including, but not limited to idiopathic distal small fiber neuropathy. Other causes of neuropathic pain that can be treated by the compounds and methods described herein include, but are not limited to, exposure to toxins or drugs (e.g., arsenic, thallium, alcohol, chemotherapeutic agents such as vincristine and cisplatin, and dideoxynucleosides), dietary or absorption abnormalities, immuno-globulinemias, hereditary abnormalities, and amputations (including mastectomy). Neuropathic pain can also result from compression of nerve fibers, such as radiculopathies and carpal tunnel syndrome.

The compounds can also be used to treat one or more of the following conditions or disorders: chronic nerve injury, chronic pain syndromes, ischemia following transient or permanent vessel occlusion, seizures, spreading depression, restless leg syndrome, hypocapnia, hypercapnia, diabetic ketoacidosis, fetal asphyxia (optionally during parturition), spinal cord injury, status epilepticus, concussion, migraine, hypocapnia, hyperventilation, lactic acidosis, or retinopathies.

In some embodiments, the use of the compounds reduces symptoms of neuropathic pain, stroke, traumatic brain injury, epilepsy, and other neurologic events or neurodegeneration resulting from NMDAR activation.

### B. Dosing and Administration

The compound is administered for a sufficient time period to alleviate the undesired symptoms and the clinical signs associated with the neurological condition or disorder being treated. In some embodiments, the compound is administered less than three times daily. In some embodiments, the compound is administered once or twice daily. In some embodiments, the compound is administered once daily. In some embodiments, the compound is administered in a single oral dosage once a day.

In some embodiments, the compound can be administered in combination with one or more additional pharmaceutically active agents, such as other medications for neurological diseases or disorders. The one or more additional pharmaceutically active agents can be formulated in the same pharmaceutical formulation as the compound. Alternatively, the one or more additional pharmaceutically active agents can be formulated in separate pharmaceutical formulation(s).

### Examples

The examples below described studies to generate NMDAR GluN2-selective negative modulators having decreased lipophilicity, reduced molecular weight, and/or improved metabolic stability. For instance, molecular size is one of the important parameters that govern transmembrane diffusion: smaller molecules (<500 g/mol) have better passive diffusion in vivo (Yang, et al., Methods in Molecular Biology, 2015, 1266:29-53).

### General Chemistry Procedures

All the commercially available chemicals were purchased from Sigma-Aldrich, Alfa Aesar, or Fisher Scientific and used without further purification. Reaction progress was monitored by either thin layer chromatography (TLC) using silica-precoated glass plates (Merck KGaA; silica gel 60 F₂₅₄, 0.25 mm thickness) or liquid chromatography-mass spectrometry (LC-MS) on an Agilent Technologies 6100 quadrupole instrument equipped with UV detection at 254 and 210 nm and Agilent C18 XDB eclipse column (50 mm×4.6 mm, 3.5 μM). Automated flash column chromatography was performed using a Teledyne ISCO CombiFlash Companion system with silica gel-packed columns (RediSep). NMR spectra (¹H, ¹³C, ¹⁹F, and ³¹P) were obtained using either a Varian INOVA 600 MHz spectrometer, a Bruker 600 MHz spectrometer, a Varian INOVA 500 MHz spectrometer, a Varian VNMR 400 MHz spectrometer, or a Bruker 400 MHz spectrometer. ¹H NMR samples were prepared and processed in deuterated chloroform (CDCl₃) or deuterated methanol (CD₃OD) using the corresponding solvent peak as a reference, while ¹⁹F NMR spectra used the trifluoroacetic acid (TFA) residual peak as a reference. NMR data were reported to include chemical shifts (6) reported in ppm, multiplicities indicated as s (singlet), d (doublet), t (triplet), q (quartet), p (pentet), hept (heptet), td (triplet of doublets), m (multiplet), or br (broad), coupling constants (J) reported in Hz, and integration normalized to 1 atom (H, C, or F). High-resolution mass spectrometry (HRMS) was performed on a VG 70-S or JEOL instrument.

### Example 1. Design and Synthesis of NMDAR GluN2-Selective Negative Modulators with a Monocyclic Core

### A. Synthetic Procedures

A series of compounds containing a monocyclic core (e.g., compounds 15, 29, 73, 28, 55, and 56) were designed and synthesized using the procedures described below.

Synthesis of the 3-acetyl monocyclic intermediates containing a pyridine (for compounds 12, 39, and 68) or 2-pyridone (for compound 46) core is shown in Scheme 1.

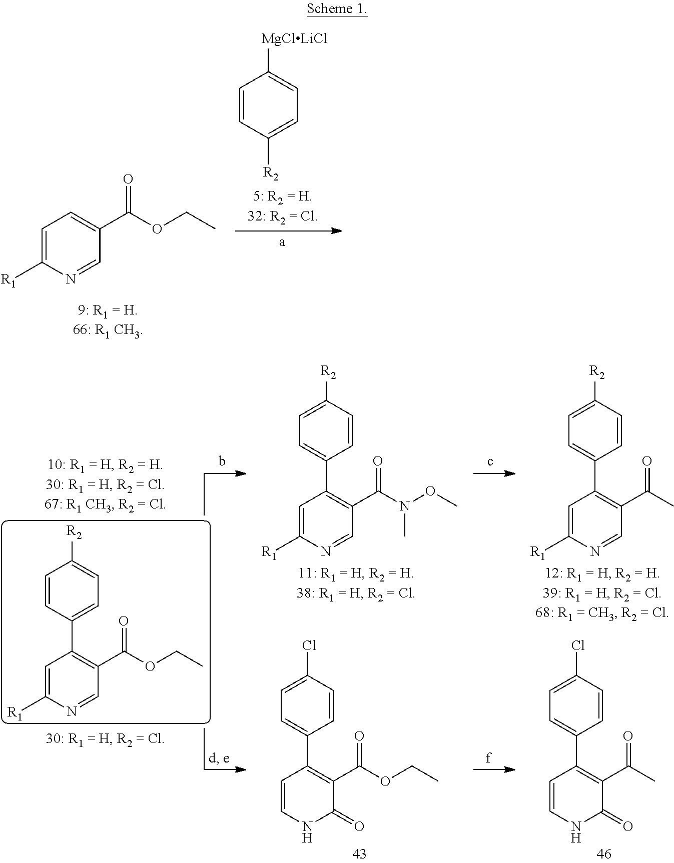

Synthesis of intermediates for the monocyclic-core analogs. Reagents and reactions for (a): (i) BF₃-OEt₂, THF, 0° C., 15 min; (ii) 5 or 32, −30° C., 2 h; (iii) chloranil, rt, 12 h, 37-79% over three steps. Reagents and reactions for (b): iPrMgCl, N,O-dimethylhydroxylamine, THF, 0° C., 30 min, 46%. Reagents and reactions for (c): MeMgBr, THF, 0° C., 1 h, 74%. Reagents and reactions for (d): m-CPBA, DCM, rt, 30 min, 65%. Reagents and reactions for (e): acetic anhydride, 130° C., 3.5 h, 42%. Reagents and reactions for (f): MeLi, THF, −78° C., 30 min, TMSCl, 46%.

Commercially available ethyl nicotinate (9 or 66) was first activated by Lewis acid (BF₃-Et₂O) and then treated with phenyl magnesium chloride (7 or 32) to generate the ethyl pyridine-3-carboxylate intermediate (10, 30, and 67) (Chen, et al., J. Am. Chem. Soc., 2013, 135(13):4958-61). Conversion of the ethyl pyridine-3-carboxylate intermediate to the desired 3-aceyl pyridine intermediate (12, 39, and 68) was achieved via the Weinreb ketone synthesis. The ethyl ester was treated with N,O-dimethylhydroxylamine to afford the Weinreb amide (11 and 38), followed by treatment with methyl magnesium bromide to yield the methyl ketone (12, 39, and 68). Oxidation of the ethyl pyridine-3-carboxylate intermediate (30) by m-CPBA followed by Polonovski rearrangement gave the 4-phenyl-2-pyridone-3-carboxylate intermediate (43) (Hyroyuki, et al., Chemical and Pharmaceutical Bulletin, 1987, 35(10):9), which was then converted to 3-acetal-2-pyridone intermediate (46) by treatment with methyllithium (Cooke, J. Org. Chem., 1986, 51(6)).

Synthesis of 3-acetyl-5,6-substituted 2-pyridone intermediates (for compounds 90 and 102) was achieved via the Knoevenagel reaction (Rai, et al., RSC Adv., 2014, 4(83):44141-44145) as shown in Scheme 2.

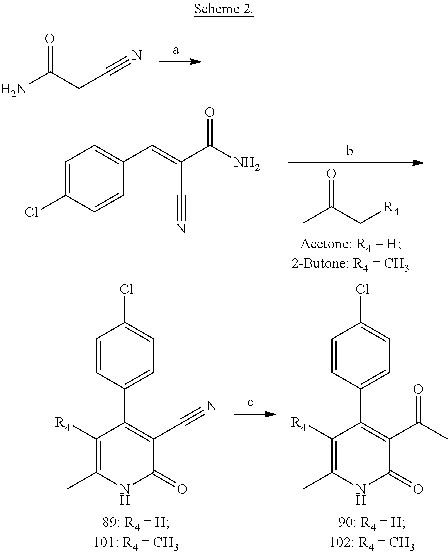

Synthesis of 3-acetyl 2-pyridone intermediates for the substituted 2-pyridone analogs. Reagents and conditions for (a): 4-chloro-benzaldehyde, 1 or 2 drops of piperadine, microwave, 120° C., 1 min EtOH, 9700, Reagents and conditions for (b): appropriate ketone, NaOEt, EtOH, rt, overnight, 23%. Reagents and conditions for (c): MeMgBr (3M ethereal solution), toluene, reflux, 4 h, 42-96%.

With the intermediates, the final compounds were synthesized by following the synthetic route reported in Acker, et al., J. Med. Chem., 2013, 56(16):6434-56. See Scheme 3 for details. Aldol condensation of 3-acetyl monocyclic core intermediate (12, 39, 68, 46, 90, and 102) with 4-chloro benzaldehyde yielded unsaturated ketone intermediates (13, 40, 70, 51, 91, and 103). Then cyclization using hydrazine monohydrate followed by acyl chain installation using difluoro succinic anhydride yielded the desired analogs (15, 29, 73, 28, 55, and 56).

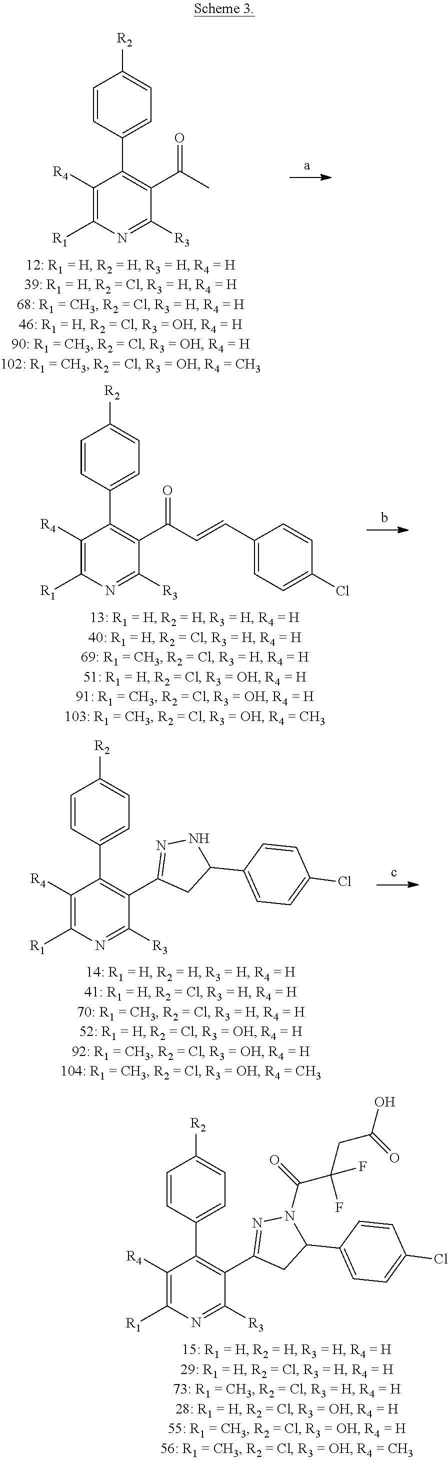

Synthesis of the monocyclic-core analogs. Reagents and conditions for (a): 4-chloro-benzaldehyde, KOH, EtOH/H₂O (3:2), 0° C. to rt, 20-74%. Reagents and conditions for (b): hydrazine monohydrate, EtOH, 110° C., microwave, 48-97%. Reagents and conditions for (c): 2,2-difluoro succinic anhydride, THF, rt, 3 h, 22-63%.

### B. Chemical Characterizations

### 1. 4-[3-(4-Chlorophenyl)-5-(4-phenyl-3-pyridyl)-3,4-dihydropyrazol-2-yl]-3,3-difluoro-4-oxo-butanoic acid (15)

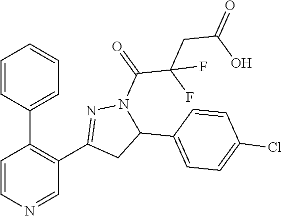 [SMILES: O=C(O)CC(F)(F)C(=O)N1N=C(c2cnccc2-c2ccccc2)CC1c1ccc(Cl)cc1]

¹H NMR (399 MHz, chloroform-d) δ 8.93 (s, 1H), 8.66 (d, J=4.9 Hz, 1H), 7.53-7.33 (m, 2H), 7.30 (t, J=7.4 Hz, 2H), 7.24 (d, J=7.4 Hz, 1H), 7.17 (d, J=8.4 Hz, 2H), 6.99 (d, J=8.1 Hz, 2H), 5.44 (dd, J=11.7, 4.4 Hz, 1H), 3.78-3.36 (m, 2H), 3.08 (dd, J=18.2, 11.7 Hz, 1H), 2.28 (dd, J=18.2, 4.4 Hz, 1H). ¹³C NMR (151 MHz, chloroform-d) δ 171.19, 169.60, 160.00 (t, J=30.8 Hz), 155.38, 150.43, 149.27, 148.60, 138.17, 137.31, 133.75, 129.45, 129.16, 128.98, 128.31, 127.02, 126.90, 125.33, 114.50 (t, J=249.7 Hz), 60.77, 43.25, 42.07 (t, J=26.2 Hz), 29.66. ¹⁹F NMR (376 MHz, chloroform-d) δ −98.41 (t, J=14.8 Hz), −98.22 (t, J=15.2 Hz), −98.61 (t, J=14.8 Hz), −99.33 (t, J=14.8 Hz). HRMS (ESI) m/z calculated for C₂₄H₁₉C₁F₂N₃O₃ [M+H]⁺: 470.87, found: 470.10782.

### 2. 4-[3-(4-Chlorophenyl)-5-[4-(4-chlorophenyl)-3-pyridyl]-3,4-dihydropyrazol-2-yl]-3,3-difluoro-4-oxo-butanoic acid (29)

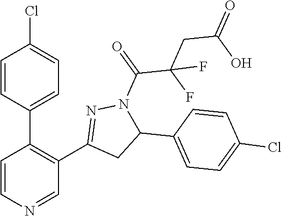 [SMILES: O=C(O)CC(F)(F)C(=O)N1N=C(c2cnccc2-c2ccc(Cl)cc2)CC1c1ccc(Cl)cc1]

¹H NMR (400 MHz, chloroform-d) δ 8.74 (s, 1H), 8.58 (d, J=5.1 Hz, 1H), 7.35-7.17 (m, 5H), 7.12 (d, J=8.4 Hz, 2H), 6.97 (d, J=8.5 Hz, 2H), 5.43 (dd, J=11.5, 4.2 Hz, 1H), 3.24 (tq, J=15.6, 4.9, 4.2 Hz, 3H), 2.42 (dd, J=18.1, 4.2 Hz, 1H). ¹³C NMR (126 MHz, chloroform-d) δ 168.60, 160.04 (t, J=30.9 Hz), 155.59, 150.40, 149.20, 148.58, 138.01, 136.04, 135.38, 133.76, 129.56, 129.19, 129.08, 128.96, 126.71, 126.19, 125.12, 114.52 (t, J=250.0 Hz), 60.59, 49.60, 43.58, 41.21 (t, J=26.1 Hz). ¹⁹F NMR (376 MHz, chloroform-d) 6-94.09-−107.15 (m). HRMS (ESI) m/z calculated for C₂₀H₁₆Cl₂N₃ [M+H]⁺: 504.06, found: 504.06884.

### 3. 4-[3-(4-Chlorophenyl)-5-[4-(4-chlorophenyl)-6-methyl-3-pyridyl]-3,4-dihydropyrazol-2-yl]-3, 3-difluoro-4-oxo-butanoic acid (73)

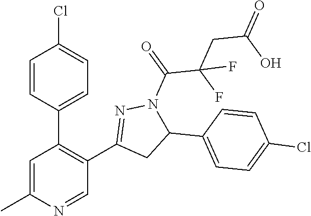 [SMILES: Cc1cc(-c2ccc(Cl)cc2)c(C2=NN(C(=O)C(F)(F)CC(=O)O)C(c3ccc(Cl)cc3)C2)cn1]

¹H NMR (500 MHz, chloroform-d) δ 8.61 (s, 1H), 7.24 (dd, J=8.1, 1.2 Hz, 4H), 7.14-7.07 (m, 3H), 7.00-6.95 (m, 2H), 5.43 (dd, J=11.6, 4.2 Hz, 1H), 3.27-3.12 (m, 1H), 2.98 (dd, J=36.8, 16.2 Hz, 1H), 2.66 (dd, J=16.4, 8.1 Hz, 1H), 2.56 (s, 3H), 2.45 (dd, J=18.1, 4.3 Hz, 1H).

### 4. 4-[3-(4-Chlorophenyl)-5-[4-(4-chlorophenyl)-2-oxo-1H-pyridin-3-yl]-3,4-dihydropyrazol-2-yl]-3,3-difluoro-4-oxo-butanoic acid (28)

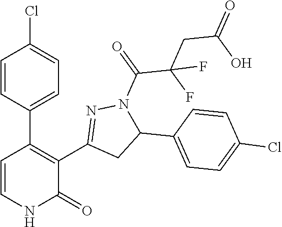 [SMILES: O=C(O)CC(F)(F)C(=O)N1N=C(c2c(-c3ccc(Cl)cc3)cc[nH]c2=O)CC1c1ccc(Cl)cc1]

¹H NMR (399 MHz, chloroform-d) δ 7.32 (d, J=6.6 Hz, 1H), 7.30-7.25 (m, 2H), 7.17 (d, J=8.3 Hz, 2H), 7.12 (d, J=8.4 Hz, 2H), 6.94 (d, J=8.4 Hz, 2H), 6.23 (d, J=7.0 Hz, 1H), 5.38 (dd, J=11.7, 4.3 Hz, 1H), 3.65 (dd, J=18.4, 11.8 Hz, 1H), 3.34 (d, J=0.8 Hz, 2H), 2.83 (dd, J=18.4, 4.5 Hz, 1H).

### 5. 4-[3-(4-Chlorophenyl)-5-[4-(4-chlorophenyl)-6-methyl-2-oxo-1H-pyridin-3-yl]-3,4-dihydropyrazol-2-yl]-3,3-difluoro-4-oxo-butanoic acid (55)

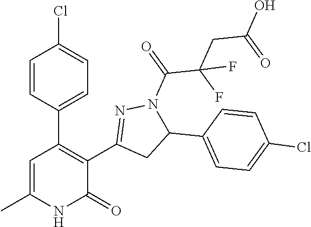 [SMILES: Cc1cc(-c2ccc(Cl)cc2)c(C2=NN(C(=O)C(F)(F)CC(=O)O)C(c3ccc(Cl)cc3)C2)c(=O)[nH]1]

¹H NMR (400 MHz, chloroform-d) δ 7.29 (d, J=8.3 Hz, 2H), 7.20 (d, J=8.2 Hz, 2H), 7.13 (d, J=8.4 Hz, 2H), 6.97 (d, J=8.5 Hz, 2H), 6.10 (s, 1H), 5.41 (d, J=10.4 Hz, 1H), 3.76-3.55 (m, 2H), 3.26-3.04 (m, 2H), 2.29 (s, 3H). ¹³C NMR (126 MHz, chloroform-d, methanol-d₄) δ 154.95, 154.88, 146.37, 138.53, 136.47, 135.14, 133.59, 129.20, 128.86, 128.81, 127.22, 109.29, 60.19, 44.48, 29.65, 18.98. ¹⁹F NMR (376 MHz, chloroform-d) δ −98.20-−101.01 (m), −104.33 (t, J=13.9 Hz). HRMS (ESI) m/z calculated for C₂₅H₂₀C₁₂F₂N₃O₄ [M+H]⁺: 534.07, found: 534.07918.

### 6. 4-[3-(4-Chlorophenyl)-5-[4-(4-chlorophenyl)-5,6-dimethyl-2-oxo-1H-pyridin-3-yl]-3,4-dihydropyrazol-2-yl]-3,3-difluoro-4-oxo-butanoic acid (56)

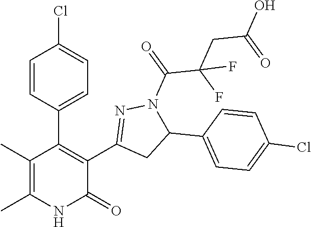 [SMILES: Cc1[nH]c(=O)c(C2=NN(C(=O)C(F)(F)CC(=O)O)C(c3ccc(Cl)cc3)C2)c(-c2ccc(Cl)cc2)c1C]

¹H NMR (399 MHz, chloroform-d) δ 7.46-7.29 (m, 2H), 7.15 (d, J=8.0 Hz, 1H), 7.09 (d, J=8.1 Hz, 1H), 6.94 (d, J=7.6 Hz, 2H), 6.80 (d, J=8.1 Hz, 2H), 5.33 (d, J=11.3 Hz, 1H), 3.58-3.35 (m, 3H), 2.59 (d, J=18.1 Hz, 1H), 2.22 (s, 3H), 1.76 (s, 3H). ¹³C NMR (151 MHz, chloroform-d, methanol-d₄) δ 156.45, 155.71, 144.26, 138.80, 135.64, 134.18, 133.28, 130.29, 128.78, 128.76, 128.70, 128.61, 128.53, 128.51, 128.41, 127.00, 126.90, 113.32, 60.15, 44.71, 29.48, 17.14, 14.00. ¹⁹F NMR (400 MHz, chloroform-d) 6-98.90 (d, J=92.8 Hz). HRMS (ESI) m/z calculated for C₂₆H₂₁Cl₂F₂N₃O₄ [M−H]⁺:546.09, found: 546.0798.

### Example 2. Design and Synthesis of NMDAR GluN2-Selective Negative Modulators with a Bicyclic Core

### A. Synthetic Procedures

A series of compounds containing a bicyclic core (e.g., compounds 57(a), 57(b), 88, and 120) were designed and synthesized using the procedures described in Scheme 4. See also Acker, et al., J. Med. Chem., 2013, 56(16):6434-56; Aurelio, et al., J. Med. Chem., 2010, 53(18):6550-6559; Whyte, et al., Angew. Chem. Int. Ed., 2018, 57(42):13927-13930.

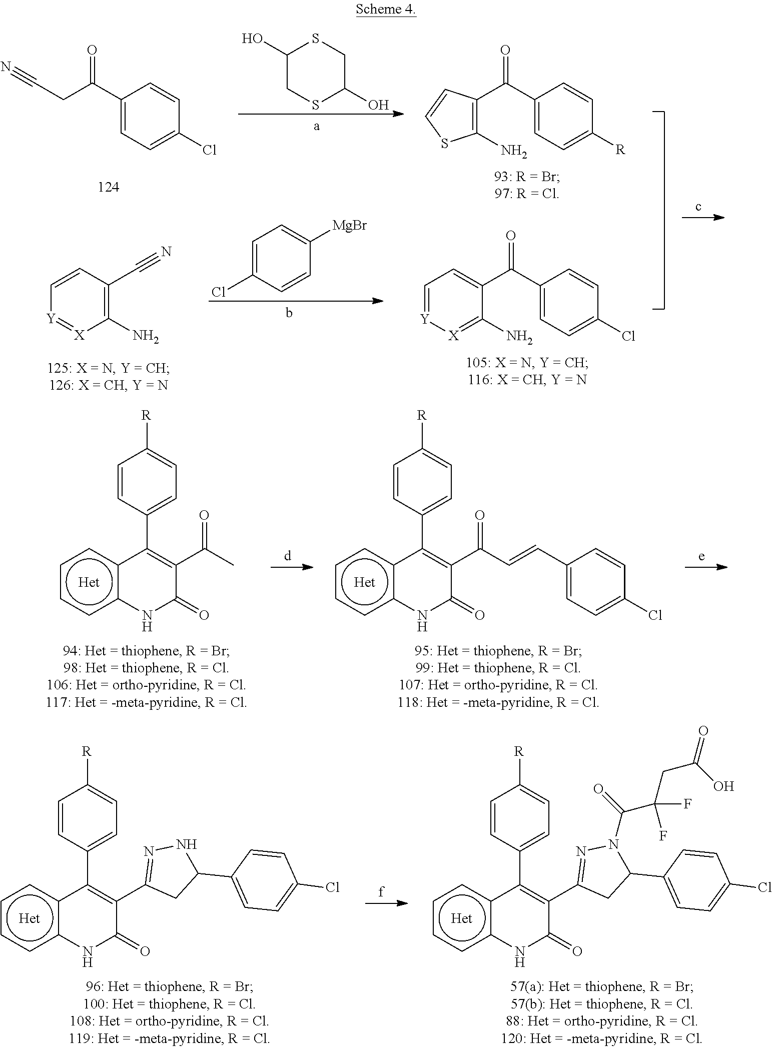

Scheme 4. Synthesis of the bicyclic-core analogs. Reagents and conditions for (a): DEA, EtOH, 50° C., 48%. Reagents and conditions for (b): 4-chlorophenyl magnesium bromide, toluene, 4 h, reflux, 34-41%. Reagents and conditions for (c): ethyl acetoacetate, DMF, drops of piperidine, 4 Å molecular sieves, 180° C., microwave, 8 min, 13-60%. Reagents and conditions for (d): 4-chloro-benzaldehyde, KOH, EtOH/H₂O (3:2), 0° C. to rt, 27-83%. Reagents and conditions for (e): hydrazine monohydrate, EtOH, 110° C., microwave, 29-94%. Reagents and conditions for (f): 2,2-difluoro-succinic anhydride, THF, rt, 3 h, 5-49%.

### B. Chemical Characterizations

### 1. 4-[5-[4-(4-Bromophenyl)-6-oxo-7H-thieno[2,3-b]pyridin-5-yl]-3-(4-chlorophenyl)-3,4-dihydropyrazol-2-yl]-3,3-difluoro-4-oxo-butanoic acid (57(a))

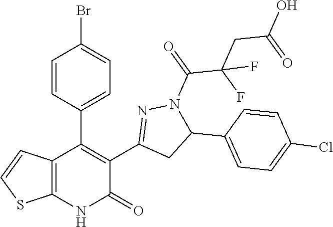 [SMILES: O=C(O)CC(F)(F)C(=O)N1N=C(c2c(-c3ccc(Br)cc3)c3ccsc3[nH]c2=O)CC1c1ccc(Cl)cc1]

¹H NMR (400 MHz, chloroform-d) δ 7.57 (m, 3H), 7.20-7.07 (m, 3H), 6.97 (d, J=5.7 Hz, 1H), 6.83 (s, 2H), 6.69 (d, J=5.7 Hz, 1H), 5.44 (s, 1H), 3.44 (m, 3H), 2.54 (s, 1H). HRMS (ESI) m/z calculated for C₂₆H₁₇BrClF₂N₃O₄S [M−H]⁻: 617.98, found: 617.97154.

### 2. 4-[3-(4-Chlorophenyl)-5-[4-(4-chlorophenyl)-6-oxo-7H-thieno[2,3-b]pyridin-5-yl]-3,4-dihydropyrazol-2-yl]-3,3-difluoro-4-oxo-butanoic acid (57(b))

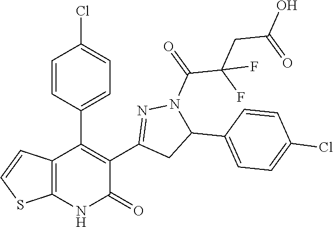 [SMILES: O=C(O)CC(F)(F)C(=O)N1N=C(c2c(-c3ccc(Cl)cc3)c3ccsc3[nH]c2=O)CC1c1ccc(Cl)cc1]

¹H NMR (399 MHz, chloroform-d) δ 7.31 (m, 2H), 7.15-7.10 (m, 3H), 7.06 (s, 1H), 6.88 (d, J=5.8 Hz, 1H), 6.81 (d, J=8.4 Hz, 2H), 6.58 (d, J=5.8 Hz, 1H), 5.32 (d, J=9.8 Hz, 1H), 3.64 (dd, J=18.4, 11.5 Hz, 1H), 3.10 (q, J=15.6, 15.2 Hz, 2H), 2.72 (dd, J=18.4, 4.1 Hz, 1H). ¹³C NMR (126 MHz, methanol-d₄) δ 168.24, 161.36, 159.62 (t, J=30.8 Hz), 155.91, 148.77, 148.66, 138.49, 135.05, 134.39, 133.55, 130.30, 129.41, 128.87, 128.77, 127.10, 122.79, 122.40, 118.48, 114.36, 60.31, 44.72, 29.60. ¹⁹F NMR (400 MHz, chloroform-d) δ−99.06-−101.53 (m).

### 3. 4-[3-(4-Chlorophenyl)-5-[4-(4-chlorophenyl)-2-oxo-1H-1,8-naphthyridin-3-yl]-3,4-dihydropyrazol-2-yl]-3,3-difluoro-4-oxo-butanoic acid (88)

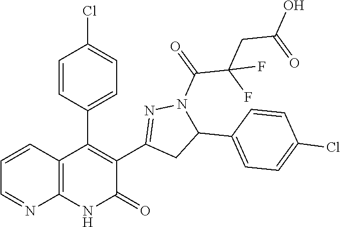 [SMILES: O=C(O)CC(F)(F)C(=O)N1N=C(c2c(-c3ccc(Cl)cc3)c3cccnc3[nH]c2=O)CC1c1ccc(Cl)cc1]

¹H NMR (600 MHz, chloroform-d) δ 8.62-8.47 (m, 1H), 7.53-7.46 (m, 1H), 7.44 (dd, J=8.2, 2.2 Hz, 1H), 7.26 (dd, J=8.2, 2.2 Hz, 1H), 7.15 (d, J=8.1 Hz, 2H), 7.11 (dd, J=8.1, 4.6 Hz, 1H), 7.02 (dd, J=8.2, 2.2 Hz, 1H), 6.74 (d, J=8.2 Hz, 2H), 5.36 (dd, J=11.7, 4.4 Hz, 1H), 3.71 (dd, J=18.5, 11.8 Hz, 3H), 2.67 (dd, J=18.5, 4.2 Hz, 1H). ¹³C NMR (126 MHz, chloroform-d) δ 168.73, 164.97, 159.63, 51.77, 150.48, 148.73, 138.32, 136.74, 135.55, 133.66, 131.83, 131.04, 129.59, 129.17, 129.04, 128.86, 127.00, 124.27, 119.35, 115.56, 113.47 (t, J=249.1 Hz), 60.42, 44.75, 40.93 (t, J=25.9 Hz). ¹⁹F NMR (376 MHz, chloroform-d) δ −98.71-−101.19 (m).

### 4. 4-[3-(4-Chlorophenyl)-5-[4-(4-chlorophenyl)-2-oxo-1H-1,7-naphthyridin-3-yl]-3,4-dihydropyrazol-2-yl]-3,3-difluoro-4-oxo-butanoic acid (120)

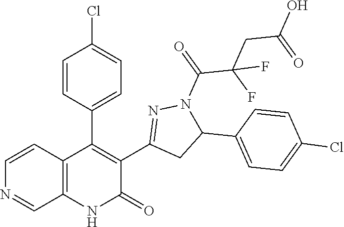 [SMILES: O=C(O)CC(F)(F)C(=O)N1N=C(c2c(-c3ccc(Cl)cc3)c3ccncc3[nH]c2=O)CC1c1ccc(Cl)cc1]

¹H NMR (400 MHz, methanol-d₄) δ 8.71 (s, 1H), 8.27 (d, J=5.4 Hz, 1H), 7.50 (dd, J=8.2, 2.2 Hz, 1H), 7.43 (dd, J=8.2, 2.2 Hz, 1H), 7.31 (dd, J=8.2, 2.2 Hz, 1H), 7.22-7.16 (m, 2H), 7.09 (ddd, J=12.6, 7.3, 3.1 Hz, 2H), 7.03 (d, J=5.5 Hz, 1H), 6.83-6.72 (m, 2H), 5.42 (dd, J=11.9, 4.3 Hz, 1H), 3.77-3.62 (m, 1H), 3.32-3.14 (m, 1H), 2.74 (dd, J=18.5, 4.3 Hz, 1H). ¹³C NMR (151 MHz, methanol-d₄) δ 168.35, 160.52, 159.83 (t, J=30.7 Hz), 154.92, 149.76, 142.04, 138.65, 138.34, 135.61, 133.96, 133.59, 131.58, 130.99, 129.68, 129.22, 129.12, 128.76, 128.34, 127.02, 124.78, 120.27, 114.42 (t, J=250.3 Hz), 60.51, 44.76, 41.00 (t, J=25.5 Hz). ¹⁹F NMR (376 MHz, methanol-d₄) δ −100.17 (ddd, J=121.0, 17.2, 13.0 Hz). HRMS (ESI) m/z calculated for C₂₇H₁₉C₁₂F₂N₄O₄ [M+H]⁺:571.07, found: 571.0754.

### Example 3. Design and Synthesis of NMDAR GluN2-Selective Negative Modulators without the Carboxylate Tail

### A. Synthetic Procedures

A series of compounds without the carboxylate tail (e.g., compounds 109 and 123) were designed and synthesized using the procedures shown in Scheme 5.

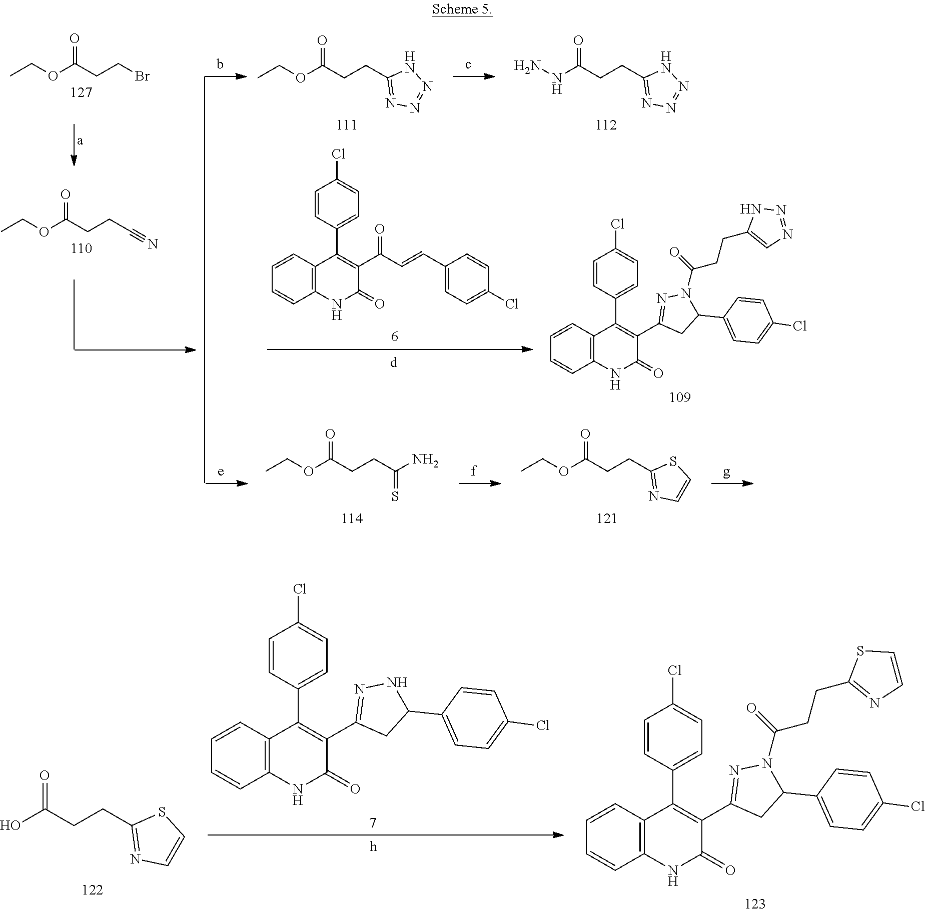

Synthesis of tetrazole/thiazole analogs. Reagents and conditions for (a): NaCN, DMSO, 50° C., 3 h, then rt, 15 h, 55%. Reagents and conditions for (b): NaN₃, triethylammonium chloride, nitrobenzene, microwave, 100° C., 2 h, 99%. Reagents and conditions for (c): hydrazine monohydrate, EtOH, microwave, 115° C., 5 min, 70%. Reagents and conditions for (d): 6, K₂CO₃, EtOH, microwave, 110° C., 20 min, 4%. Reagents and conditions for (e): P₂S₅, EtOH, rt, overnight, 15%. Reagents and conditions for (f): 2-chloro-1,1-diethoxyethane, pTSA, acetic acid, 90° C., 90 min, 25%. Reagents and conditions for (g): NaOH, EtOH, rt, overnight, 47%. Reagents and conditions for (h): i) oxlyl chloride, 2 drops of DMF, rt, 1 h; ii) 7, DCM, rt, 3 h, 8%.

The tetrazole-containing analog 109 was synthesized as described below. Tetrazole intermediate 111 was synthesized by first converting the commercially available ethyl 3-bromopropanoate 127 to ethyl 3-cyanopropanoate 110 using sodium cyanide. The nitrile 110 was then cyclized with sodium azide to form tetrazole moiety 111 under microwave condition. The ethyl ester 111 was converted to the monosubstituted hydrazide 112 using hydrazine monohydrate under microwave radiation (Ruger, et al., ChemMedChem, 2015, 10(11):1875-1983). Condensation of the hydrazide 112 with dihydroquinoline acryloyl intermediate 6 in the presence of K₂CO₃ afforded the final tetrazole analog 109.

The thiazole-containing analog 123 was synthesized as described below. The synthetic route of 123 was similar to that of 109. Nitrile 110 was treated with phosphorus pentasulfide to afford thioamide 114, which was then cyclized with chloroacetaldehyde diethyl acetal to form the thiazole ring (compound 121). Hydrolysis of the ethyl ester in compound 121 yielded the corresponding acid (compound 122), which was then activated by oxalyl chloride to form acyl chloride and then replaced by pyrazoline intermediate 7 to afford the final thiazole analog 123.

### B. Chemical Characterizations

### 1. 4-(4-Chlorophenyl)-3-[3-(4-chlorophenyl)-2-[3-(1H-tetrazol-5-yl)propanoyl]-3,4-dihydropyrazol-5-yl]-1H-quinolin-2-one (109)

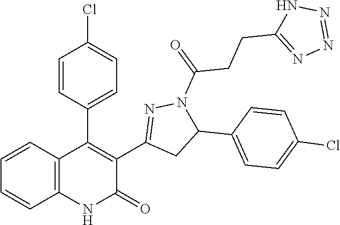 [SMILES: O=C(CCc1nnn[nH]1)N1N=C(c2c(-c3ccc(Cl)cc3)c3ccccc3[nH]c2=O)CC1c1ccc(Cl)cc1]

¹H NMR (600 MHz, methanol-d₄) δ 7.57 (t, J=7.8 Hz, 1H), 7.51 (d, J=8.2 Hz, 1H), 7.42 (d, J=8.2 Hz, 1H), 7.36 (t, J=9.0 Hz, 2H), 7.14 (dt, J=26.8, 7.7 Hz, 5H), 6.67 (d, J=7.8 Hz, 2H), 5.32 (dd, J=11.9, 3.3 Hz, 1H), 3.71 (dd, J=18.4, 11.8 Hz, 1H), 3.12 (t, J=7.2 Hz, 2H), 3.03 (q, J=7.4, 6.9 Hz, 2H), 2.67-2.57 (m, 1H). ¹³C NMR (151 MHz, methanol-d₄) δ 169.25, 161.27, 155.84, 153.66, 151.70, 139.53, 138.28, 134.82, 133.54, 133.36, 131.89, 131.24, 129.94, 128.87, 128.79, 128.71, 128.60, 127.71, 126.85, 123.11, 122.82, 119.98, 115.80, 59.18, 45.67, 31.43, 28.80. HRMS (ESI) m/z calculated for C₂₈H₂₂C₁₂N₇O₂ [M+H]⁺: 557.11, found: 558, 12219.

### 2. 4-(4-Chlorophenyl)-3-[3-(4-chlorophenyl)-2-(3-thiazol-2-ylpropanoyl)-3,4-dihydropyrazol-5-yl]-1H-quinolin-2-one (123)

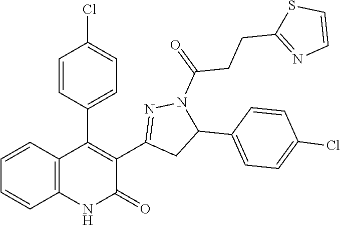 [SMILES: O=C(CCc1nccs1)N1N=C(c2c(-c3ccc(Cl)cc3)c3ccccc3[nH]c2=O)CC1c1ccc(Cl)cc1]

¹H NMR (600 MHz, chloroform-d) δ 7.45 (ddd, J=8.3, 6.3, 2.1 Hz, 1H), 7.37 (dd, J=8.1, 2.2 Hz, 1H), 7.29 (dd, J=8.2, 2.2 Hz, 1H), 7.26-7.23 (m, 2H), 7.23-7.21 (m, 1H), 7.13-7.08 (m, 3H), 7.05-7.00 (m, 3H), 6.71-6.65 (m, 2H), 5.24 (dd, J=11.9, 4.3 Hz, 1H), 3.65 (dd, J=18.4, 11.9 Hz, 1H), 3.15 (td, J=7.4, 3.6 Hz, 2H), 2.90 (dt, J=16.7, 7.5 Hz, 1H), 2.84-2.77 (m, 1H), 2.74 (dd, J=18.4, 4.3 Hz, 1H). ¹³C NMR (151 MHz, methanol-d₄) δ 169.97, 169.33, 161.41, 153.06, 151.53, 141.77, 139.70, 138.15, 134.69, 133.62, 133.28, 131.78, 131.18, 129.99, 128.75, 128.58, 128.52, 127.80, 127.03, 123.07, 123.05, 120.06, 118.59, 115.78, 59.14, 45.47, 33.60, 27.63. HRMS (ESI) m/z calculated for C₁₆H₁₂ClN₂O₂ [M+H]⁺:573.08, found: 573.0924.

### Example 4. Biological Activity of the NMDAR Modulators

### A. Methods

Two-electrode voltage-clamp recording was performed in Xenopus laevis oocytes injected with mRNA to express recombinant rat GluN1/GluN2A, GluN1/GluN2B, GluN1/GluN2C, and GluN1/GluN2D. cDNAs for rat NMDA subunits GluN1-la (GenBank U08261; hereafter GluN1), GluN2A (GenBank D13211), GluN2B (GenBank U11419), GluN2C (GenBank M91563), and GluN2D (GenBank L31611) were provided by Drs. S. Heinemann from Salk Institute, S. Nakanishi from Kyoto University, and P. Seeburg from the University of Heidelberg. An automatic injector (Nanoject II, Drummond Scientific) was used for cRNA injection using the pipettes filled with mineral oil. The cRNA that transcribed in vitro via the mMessage Machine kit (Ambion) was diluted with nuclease-free water, and then injected at a GluN1/GluN2 ratio of 1:2. The oocytes were stored in Barth's solution that contained 88 mM NaCl, 5.0 mM Tris-HCl, 2.4 mM NaHCO₃, 1.0 mM KCl, 0.84 mM MgSO₄, 0.41 mM CaCl₂), 0.33 mM Ca(NO₃)₂, 0.1 mg/ml gentamycin sulfate, 1.0 U/ml penicillin, and 1 μg/ml streptomycin at pH 7.4 and temperatures of 15-17° C. for two to five days before the two-electrode voltage-clamp recordings.

During recording, the oocytes were placed in a perfusion chamber and continually washed with the recording solution containing 90 mM NaCl, 1.0 mM KCl, 0.50 mM BaCl₂, 0.005 mM EDTA, and 10 mM HEPES at pH 7.4 and a temperature of 23° C. Glass electrodes with a tip resistance of 0.5 to 2.5 MΩ were obtained from thin-walled glass capillary tubes. Voltage electrodes and current electrodes were filled with 0.3 M and 3.0 M KCl, respectively. The recordings were executed with the oocytes membrane potential holding at −40 mV by an OC-725 amplifier (Warner Instrument Co.). All compounds were dissolved in dimethyl sulfoxide (DMSO) as 20 mM stock solutions and further diluted to reach the desired concentration (0.05-0.5% (vol/vol) DMSO) in recording solution comprised of 30 □M glycine and 100 □M glutamate. In general, each compound was tested in multiple oocytes. To evaluate the inhibitory efficacy of the compounds, concentration-response curves were plotted using the average two-electrode voltage-clamp recording results and fitted by the following equation:

response=100/{1+[(inhibitor concentration)/IC₅₀]^{N}}
where IC₅₀ is the concentration of compound that inhibit half maximal of the current response and N is the Hill slope.

### B. Results

Table 1 summarizes the activity of compounds 15, 29, 73, 28, 55, and 56 against GluN2 NMDARs. Table 2 summarizes the activity of compounds 57(a), 57(b), 88, and 120 against GluN2 NMDARs. Table 3 summarizes the activity of compounds 109, 200, 300, 400, and 123 against GluN2 NMDARs.

TABLE 1
Activities against GluN2 NMDARs for the monocyclic-core analogs.

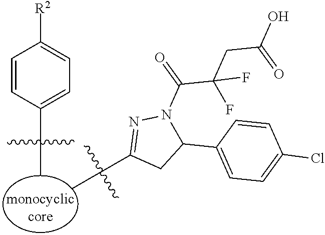

| | | | Subunit | | | | | | | | | |
|---|---|---|---|---|---|---|---|---|---|---|---|---|
| | | | Selectivity | | | | | | | | | |
| | | | 2A | 2B | | | | | | | | |
| | | | IC₅₀/ | IC₅₀/ | | | | | IC₅₀ (μM) | | | |
| | | | 2D | 2D | I₃₀ μM/I_{control} (mean ± SEM, %) | | | | [95% Confidence Interval] | | | |
| Compound | Monocyclic | R¹ | IC₅₀ | IC₅₀ | GluN2A | GluN2B | GluN2C | GluN2D | GluN2A | GluN2B | GluN2C | GluN2D |
| 15 (125-PM) | 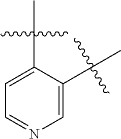 [SMILES: *c1ccncc1*] | H | — | — | 98 ± 4.1 (n = 1, 5) | 82 ± 1.1 (n = 2, 5) | 91 ± 3.5 (n = 1, 4) | 74 ± 7.1 (n = 1, 4) | — | — | — | — |
| 29 (127-PM) | 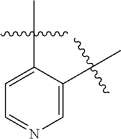 [SMILES: *c1ccncc1*] | Cl | — | — | 74 ± 3.4 (n = 1, 6) | 79 ± 4.3 (n = 1,5) | 35 ± 0.84 (n = 1, 6) | 27 ± 2.3 (n = 1, 6) | — | — | 15 [12, 19] | 12 [7.8, 17] |
| 73 (139-PM) | 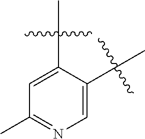 [SMILES: *c1cnc(C)cc1*] | Cl | — | — | 100 ± 2.0 (n = 2, 5) | 100 ± 2.7 (n = 1, 4) | 111 ± 1.9 (n = 1, 4) | 110 ± 3.1 (n = 1, 4) | — | — | — | — |
| 28 (136-PM) | 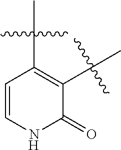 | Cl | — | — | 104 ± 2.2 (n = 1, 6) | 96 ± 2.4 (n = 1, 4) | 61 ± 1.0 (n = 1, 5) | 39 ± 1.7 (n = 1, 6) | — | — | 55 [43, 66] | 20 [12, 27] |
| 55 (145-PM) | 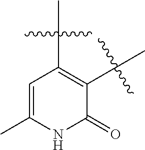 | Cl | — | 3 | 86 ± 4.4 (n = 1, 6) | 55 ± 3.0 (n = 1, 7) | 29 ± 2.0 (n = 1, 6) | 26 ± 1.8 (n = 1, 6) | — | 41 [24, 56] | 14 [12, 16] | 12 [9.8, 14] |
| 56 (147-PM) | 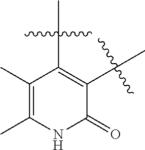 | Cl | — | — | 71 ± 5.0 (n = 1, 6) | 58 ± 2.0 (n = 2, 5) | 2.7 ± 0.32 (n = 1, 4) | 7.9 ± 2.9 (n = 1, 4) | 42 [37, 48] | 47 [40, 55] | 1.9 [1.6, 2.2] | 2.6 [2.5, 2.8] |

TABLE 2
Activities against GluN2 NMDARs for the bicyclic-core analogs.

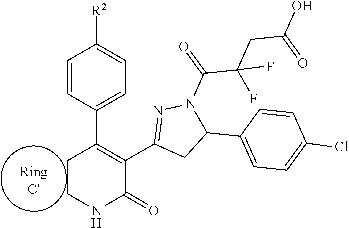

| | | | Subunit | | | | | | | | | |
|---|---|---|---|---|---|---|---|---|---|---|---|---|
| | | | Selectivity | | | | | | | | | |
| | | | 2A | 2B | | | | | | | | |
| | | | IC₅₀/ | IC₅₀/ | | | | | IC₅₀ (μM) | | | |
| | Ring | | 2D | 2D | I_{30 μM}/I_{control} (mean ± SEM, %) | | | | [95% Confidence Interval] | | | |
| Compound | C′ | R² | IC₅₀ | IC₅₀ | GluN2A | GluN2B | GluN2C | GluN2D | GluN2A | GluN2B | GluN2C | GluN2D |
| 57(a) (141-PM) | 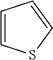 | Br | 24 | 44 | 47.6 ± 5.5 (n = 1, 3) | 37.7 ± 3.5 (n = 1, 4) | 22 ± 1.5 (n = 1, 6) | 19 ± 3.0 (n = 1, 8) | 11 [7.4, 15.2] | 19 [13.8, 23.5] | 0.56 [0.46, 0.66] | 0.43 [0.30, 0.52] |
| 57(b) (146-PM) | 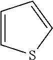 | Cl | — | — | 86 ± 7.4 (n = 1, 4) | 88 ± 4.6^{d} (n = 1, 4) | 6.9 ± 2.5 (n = 2, 4) | 5.9 ± 2.7 (n = 2, 4) | — | — | 0.32 [0.30, 0.33] | 0.25 [0.16,0 .32] |
| 88 (150-PM) | 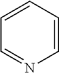 | Cl | 35 | 52 | 49 ± 7.3 (n = 2, 6) | 71 ± 0.9 (n = 2, 8) | 6.6 ± 0.9 (n = 2, 8) | 3.8 ± 0.6 (n = 2, 8) | 45 [43, 46] | 67 [59, 74] | 2.1 [1.8, 2.3] | 1.3 [1.1, 1.4] |
| 120 (155-PM) | 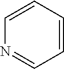 | Cl | — | — | 77 ± 4.8^{c} (n = 2, 7) | 82 ± 1.7^{c} (n = 2, 8) | 18 ± 1.0^{c} (n = 3, 11) | 7.4 ± 0.9^{c} (n = 3, 10) | — | — | 2.1 [1.9, 2.4] | 0.8 [0.6, 1.0] |

^{c} Current ratio reported for 10 μM test compound.
^{d} Current ratio reported for 3 μM test compound.

TABLE 3
Activities against GluN2 NMDARs for compounds 109, 200, 300, 400, and 123.

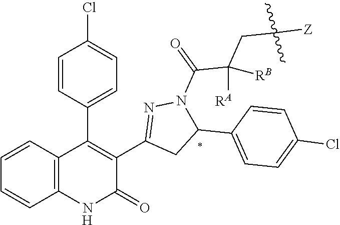

| | | | | Subunit | | | | | | | | | |
|---|---|---|---|---|---|---|---|---|---|---|---|---|---|
| | | | | Selectivity | | | | | | | | | |
| | | | | 2A | 2B | | | | | | | | |
| | | | | IC₅₀/ | IC₅₀/ | | | | | | IC₅₀ (IM) | | |
| | | R^{A} | Clog | 2D | 2D | I_{30 μM}/I_{control} (mean ± SEM, %) | | | | | [95% Confidence Interval] | | |
| Compound | Z | R^{B} | P | IC₅₀ | IC₅₀ | GluN2A | GluN2B | GluN2C | GluN2D | GluN2A | GluN2B | GluN2C | GluN2D |
| 109 (149-PM) | 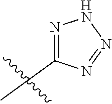 | H | 4.44 | 42 | 56 | 29 ± 4.1 (n = 2, 8) | 48 ± 1.9 (2, 8) | 2.3 ± 0.5 (2, 8) | 2.4 ± 1.6 (2, 8) | 22 [16, 28] | 29 [25, 33] | 0.57 [0.49, 0.64] | 0.52 [0.45, 0.58] |
| 200 ^{d} (997-72) | 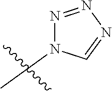 [SMILES: *n1cnnn1] | H | | — | — | 105 ± 10 (n = 1, 4) | 68 ± 1.6 (n = 1,4 ) | 20 ± 2.0 (n = 1, 3) | 18.8 ± 3.2 (n = 1, 4) | — | — | 2 | 1.7 |
| 300 ^{e} (997-87) | 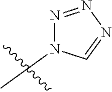 [SMILES: *n1cnnn1] | H | | — | — | 71 ± 1.8 (n = 1, 6) | 56 ± 2.9 (n = 1, 6) | 16 ± 2.8 (n = 1, 5) | 32 ± 1.5 (n = 1, 6) | — | — | 1.2 | 1.4 |
| 400 ^{f} (997-86) | 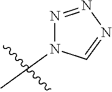 [SMILES: *n1cnnn1] | H | | — | — | 72 ± 2.5 (n = 1, 4) | 77 ± 1, 6 (n = 1, 4) | 18 ± 4.2 (n = 1, 6) | 9.1 ± 1.5 (n = 1, 6) | | | 0.95 | 0.72 |
| 123 (156-PM) | 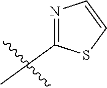 [SMILES: *c1nccs1] | H | 5.62 | — | — | 96 ± 1.8^{c} (n = 2, 8) | 94 ± 1.7^{c} (n = 2, 8) | 71 ± 4.8^{c} (n = 2, 8) | 67 ± 5.1^{c} (n = 2, 8) | — | —— | 23 [4.5, 53] | 7.9 [4.6, 12] |

^{c} Current ratio reported for 10 μM test compound.
^{d} Racemate.
^{e} (+) enantiomer.
^{f} (−) enantiomer.

What is claimed is:

1-50. (canceled)

51. A compound of Formula III, an enantiomer or diastereomer thereof, or a pharmaceutically acceptable salt thereof, 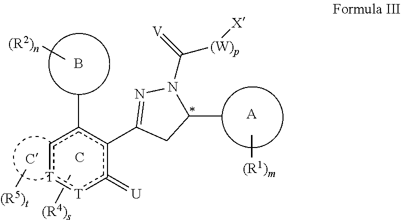
wherein ring A and ring B are independently aryl or 5- or 6-membered heteroaryl;
wherein ring C′ is aryl or 5- or 6-membered heterocyclyl;
wherein T is CH, CR⁴, CH₂, CHR⁴, C(R⁴)₂, N, NH, or NR⁴;
wherein T′ is C or N;
wherein U is O, S, or absent;
wherein V is O, S, NH, N-alkyl, two hydrogen atoms each singly bonded to the adjacent carbon, or two fluorine atoms each singly bonded to the adjacent carbon;
wherein W, at each occurrence, is independently CH₂, C(Hal)₂, CH₂CH₂, CH₂CH₂CH₂, C(Hal)₂CH₂, CH₂C(Hal)₂, CH═CH, C(Hal)=CH, CH═C(Hal), C═O, O, S, or NH, optionally substituted by one or more, the same or different R¹⁰;
wherein p is 1, 2, or 3;
wherein X′ is 5- or 6-membered nitrogen-containing heterocyclyl optionally substituted by one or more, the same or different R⁶;
wherein m and n are independently 0, 1, 2, 3, 4, or 5;
wherein s is 0, 1, or 2;
wherein t is 0, 1, 2, 3, or 4;
wherein R¹, R², R⁴, R⁵, and R⁶, at each occurrence, are individually and independently alkyl, haloalkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heteroaryl, or heterocyclyl, optionally substituted with one or more, the same or different R¹⁰;
wherein R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl; and
wherein the dotted lines in ring C each independently represent a single bond or no bond.

52. (canceled)

53. The compound of claim 51, wherein ring C′ is 5-membered heteroaryl.

54. The compound of claim 53, wherein ring C′ is thiophene, pyrrole, pyrazole, oxathiole, isoxathiole, thiazole, or isothiazole.

55. (canceled)

56. The compound of claim 51, wherein ring C′ is aryl or 6-membered heteroaryl.

57. The compound of claim 56, wherein ring C′ is phenyl, pyridine, or diazine.

58. (canceled)

59. The compound of claim 51, having a structure of Formula IIIA, an enantiomer or diastereomer thereof, or a pharmaceutically acceptable salt thereof, 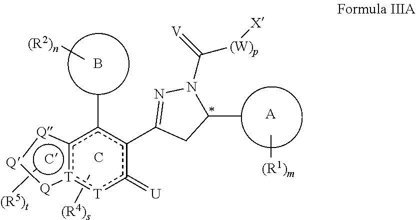
wherein Q, Q′, and Q″ are independently selected from the group consisting of S, O, N, NH, NR⁵, CH, and CR⁵;
wherein at least one of Q, Q′, and Q″ comprises a heteroatom as a ring atom for ring C′;
wherein t is 0, 1, 2, or 3;
wherein s is 0, 1, or 2; and
wherein ring A, ring B, ring C, T, T′, U, V, W, X′, R¹, R², R⁴, R⁵, m, n, and p are the same as those described for Formula III.

60. The compound of claim 59, wherein Q is S, Q′ is CH or CR⁵, and Q″ is CH or CR⁵.

61. The compound of claim 51, having a structure of Formula IIIB, an enantiomer or diastereomer thereof, or a pharmaceutically acceptable salt thereof, 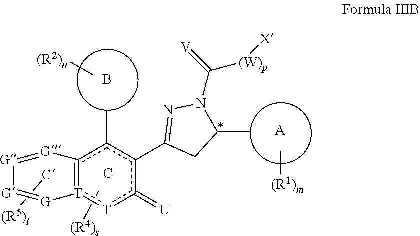
wherein G, G′, G″, and G′″ are independently CH, CR⁵ or N;
wherein t is 0, 1, 2, 3, or 4;
wherein s is 0, 1, or 2; and
wherein ring A, ring B, ring C, T, T′, U, V, W, X′, R¹, R², R⁴, R⁵, m, n, and p are the same as those described for Formula III.

62. The compound of claim 61, wherein G, G′, G″, and G′″ are independently CH or CR⁵.

63-67. (canceled)

68. The compound of claim 51, wherein ring A and ring B are phenyl.

69. The compound of claim 51, wherein V is O.

70. (canceled)

71. (canceled)

72. The compound of claim 51, wherein p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰.

73. (canceled)

74. The compound of claim 51, wherein m is 1 and R^{t} is halogen.

75. (canceled)

76. The compound of claim 51, wherein n is 1 and R² is halogen.

77. The compound of claim 51, wherein:
ring A and ring B are phenyl;
V is O;
p is 1 and W is CH₂, CF₂, CH₂CH₂, CH₂CH₂CH₂, CF₂CH₂, CH₂CF₂, CH═CH, CF═CH, or CH═CF, optionally substituted by one or more, the same or different R¹⁰; and
m and n are independently 0, 1, or 2.

78. The compound of claim 51, wherein X′ is 5-membered nitrogen-containing heterocyclyl optionally substituted by one or more, the same or different R⁶.

79. The compound of claim 78, wherein X′ is 5-membered nitrogen-containing heteroaryl optionally substituted by one or more, the same or different R⁶.

80. The compound of claim 78, having a structure of Formula IIIF, an enantiomer or diastereomer thereof, or a pharmaceutically acceptable salt thereof, 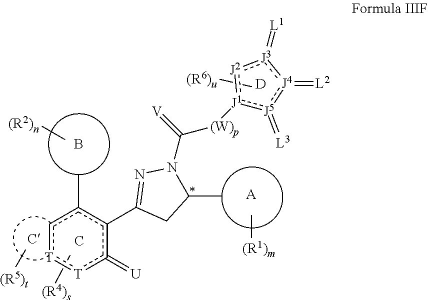
wherein u is 0, 1, 2, 3, or 4;
wherein J¹, J², J³, J⁴, and J⁵ are independently selected from the group consisting of S, O, N, NH, NR⁶, C, CH, and CR⁶;
wherein at least one of J, J², J³, J⁴, and J⁵ is N, NH, or NR^{W} and at least one of J, J², J³, J⁴ and J⁵ is C, CH, or CR⁶;
wherein L¹, L², and L³ are independently O, S, or absent;
wherein when L¹ is not absent, J³ is C or S;
wherein when L² is not absent, J⁴ is C or S;
wherein when L³ is not absent, J⁵ is C or S;
wherein the dotted lines in ring D each independently represent a single bond or no bond; and
wherein ring A, ring B, ring C, ring C′, T, T′, U, V, W, R¹, R², R⁴, R⁵, m, n, p, t, and s are the same as those described for Formula III.

81. The compound of claim 80, wherein u is 0.

82. The compound of claim 80, wherein L¹, L², and L³ are absent.

83. The compound of claim 80, wherein L¹ and L³ are absent and L² is O or S.

84. The compound of claim 80, wherein L¹ and L³ are independently O or S and L² is absent.

85. The compound of claim 51, wherein the 5- or 6-membered nitrogen-containing heterocyclyl of X′ is selected from the group consisting of tetrazole, imidazole, oxazole, triazole, thiazole, thiazolidine dione, oxazolidine dione, oxadiazol-5(4H)-one, thiadiazol-5(4H)-one, oxathiadiazole-2-oxide, and oxadiazol-5(4H)-thione.

86. (canceled)

87. The compound of claim 85, wherein the 5- or 6-membered nitrogen-containing heterocyclyl of X′ is selected from the group consisting of:

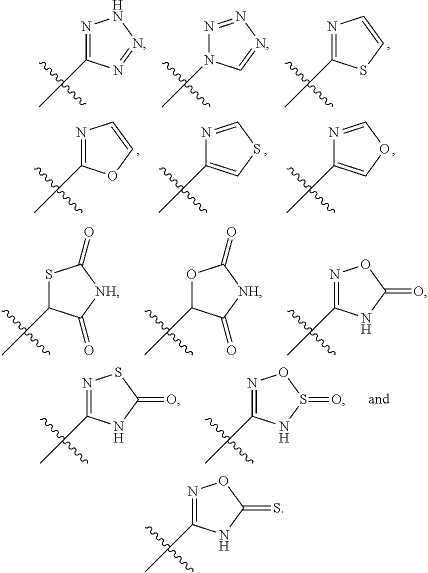

88. The compound of claim 87, wherein the compound is selected from the group consisting of: 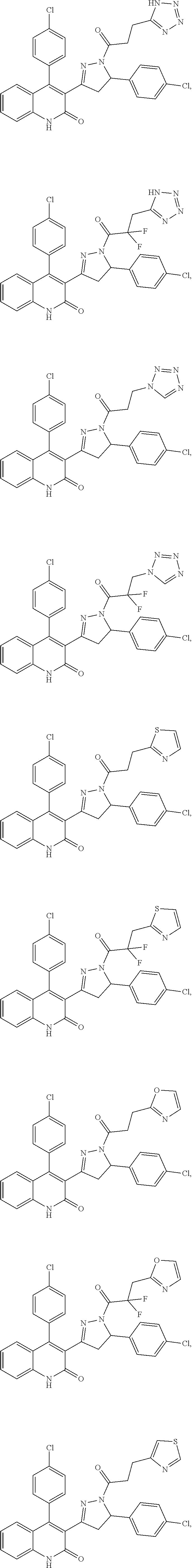 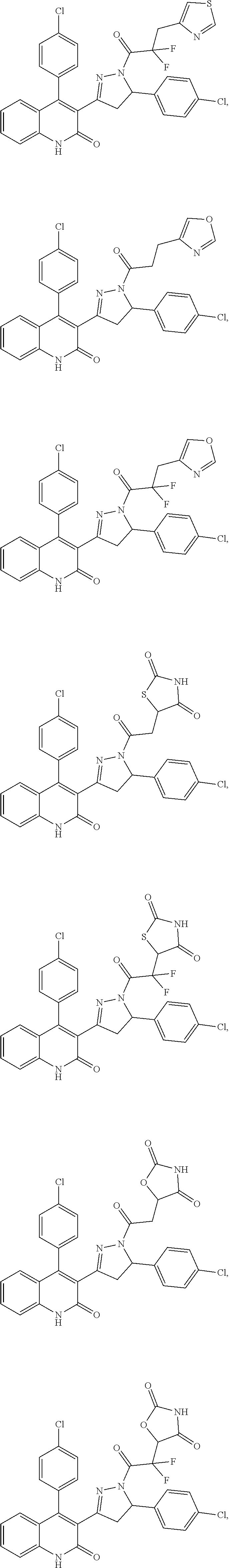 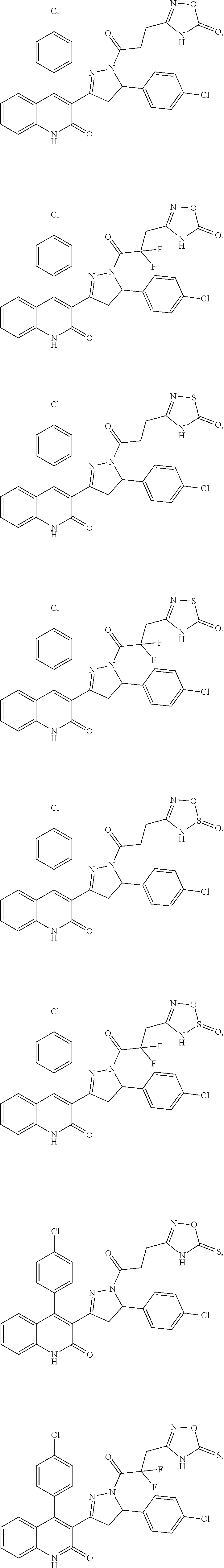
enantiomers or diastereomers thereof, and pharmaceutically acceptable salts thereof.

89. A composition, comprising the compound of claim 51, wherein the compound is in greater than 60%, 70%, 80%, 90%, 95%, or 98% enantiomeric excess with respect to the stereocenter labeled by the “*” sign in Formula III.

90. (canceled)

91. A pharmaceutical formulation, comprising the compound of claim 51 and a pharmaceutically acceptable carrier.

92. The pharmaceutical formulation of claim 91, wherein the pharmaceutical formulation is in the form of tablet, capsule, pill, gel, cream, granule, solution, suspension, emulsion, or nanoparticulate formulation.

93. The pharmaceutical formulation of claim 91, wherein the pharmaceutical formulation is an oral or intravenous formulation.

94. A method of treating a neurological condition or disorder in a subject in need thereof, comprising administering an effective amount of the compound of claim 51 to the subject.

95. The method of claim 94, wherein the compound is administered orally or intravenously.

96. The method of claim 94, wherein the neurological condition or disorder is selected from the group consisting of Parkinson's disease, dystonia and related motor disorders, depression, neuropsychiatric indications, stoke, hypoxia, traumatic brain injury, acute injuries involving the white matter, Alzheimer's disease, compulsivity and related disorders, and epilepsy.

97. (canceled)
